SEQUENCE LISTING (1 sequence)

SEQ ID NO: 1 - DNA, 59 bp, synthetic construct
  source 1..59: mol_type = other DNA
agagagcgtcgtgtggannnnnnnnnnnnnnnnnnnnnnnnntgaggaggtgcgcaagt

(19) United States
(12) Patent Application Publication
(10) Pub. No.: US 2025/0163506 A1
(43) Pub. Date: May 22, 2025

(54) ENHANCED SPATIAL PROFILING OF INTERACTIONS BETWEEN NUCLEIC ACIDS AND NUCLEIC ACID-BINDING PROTEINS

(71) Applicant: 10x Genomics, Inc., Pleasanton, CA (US)

(72) Inventors: Marco Serra, Dublin, CA (US); Justin Costa, Union City, CA (US)

(21) Appl. No.: 18/955,558

(22) Filed: Nov. 21, 2024

Related U.S. Application Data
(60) Provisional application No. 63/601,672, filed on Nov. 21, 2023.
(60) Provisional application No. 63/696,697, filed on Sep. 19, 2024.

(51) Int. Cl. C12Q 1/6874; C12Q 1/44; C12Q 1/6806

(52) U.S. Cl. CPC C12Q 1/6874; C12Q 1/44; C12Q 1/6806

(57) ABSTRACT

Systems and methods for the enhanced spatial analysis of interactions between nucleic acids and proteins in a biological sample have been developed. The methods employ a first set of nucleic acid-based probes to selectively bind target proteins in a biological sample and functionalize the nucleic acids within the sample to enable embedding within a permeable matrix, such as a gel. The methods then implement probe-based spatial analysis within the permeable matrix using a second set of probes designed to hybridize with both a target nucleic acid and with the first probes bound to the target proteins. The resulting hybridized first and second probes are bound by a capture probe including a spatial recognition barcode to provide spatial information for interactions between nucleic acids and target proteins within the sample.

### CROSS-REFERENCE TO RELATED APPLICATION

This application claims the benefit of and priority to U.S. Provisional Application No. 63/601,672, filed on Nov. 21, 2023, and U.S. Provisional Application No. 63/696,697, filed on Sep. 19, 2024, both of which are hereby incorporated herein by reference in their entireties.

### REFERENCE TO SEQUENCE LISTING

The Sequence Listing XML submitted as a file named “GENOMX_2023_149_US.xml,” created on Nov. 21, 2024, and having a size of 1,913 bytes is hereby incorporated by reference pursuant to 37 C.F.R. § 1.834(c)(1).

### FIELD OF THE INVENTION

The invention is generally in the field of molecular profiling of analytes present in a biological sample, specifically compositions, kits, methods, and systems for enhanced spatial profiling of interactions between nucleic acids and proteins.

### BACKGROUND OF THE INVENTION

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

A wide variety of methods have been developed to describe interactions between proteins and nucleic acids both in vivo and in vitro. These include chromatin immunoprecipitation, yeast one hybrid assay, microplate capture and ELISA based detection assays, filter binding assay, electrophoretic mobility shift assay (EMSA), fluorescence anisotropy, surface plasmon resonance, kinetic assays based on stopped-flow systems, and single molecule techniques. However, each method has its limitations, and a combination of several techniques are often applied for the analysis of a particular interaction. For example, EMSA is one of the most commonly used methods for determining protein/nucleic acid affinity and sequence specificity, but it is a rather laborious and time-consuming technique with relatively limited throughput that may be insensitive to low-affinity interactions.

The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and crosstalk with other cells in the tissue. Spatial heterogeneity has been previously studied using techniques that only provide data for a small handful of analytes in the context of an intact tissue or a segment of a tissue or provide a lot of analyte data for single cells but fail to provide information regarding the position of the single cell in a parent biological sample (e.g., tissue sample).

The spatial heterogeneity in developing systems has typically been studied via RNA hybridization, immunohistochemistry, fluorescent reporters, or purification or induction of pre-defined subpopulations and subsequent genomic profiling (e.g., RNA-seq). Such approaches, however, rely on a small set of pre-defined markers, therefore introducing selection bias that limits discovery and increases the cost and labor required to localize RNA on transcriptome-wide basis.

Methods for spatial profiling of analytes present in a biological sample include use of spatially barcoded substrates to detect analytes. Proximity ligation is used to identify two analytes that have a spatial relationship to one another (e.g., two sequences on the same transcript; protein-protein interaction). Methods for spatial profiling using proximity ligation are described in US Publication Number US 2021/0230681 A1.

There is a need for enhanced methods for spatially detecting protein/nucleic acid interactions, as well as detecting temporal changes in interactions between nucleic acids and proteins in biological samples.

Therefore, it is an object of the invention to provide enhanced methods for detecting the presence, location, and/or abundance of interactions between nucleic acids and proteins in a biological sample from a subject.

It is a further object of the invention to provide enhanced systems and reagents for the early detection of diseases and disorders based on the presence and/or abundance of specific protein-nucleic acid interactions within a biological sample from a subject.

It is a further object of the invention to provide enhanced methods to monitor the progression of cellular changes in the cellular micro-environment based on the presence and/or abundance of specific protein-nucleic acid interactions within a biological sample from a subject.

### SUMMARY OF INVENTION

Systems and methods for the enhanced spatial analysis of interactions between nucleic acids and proteins in a biological sample have been developed. The methods employ a first set of nucleic acid-based probes to selectively bind target proteins in a biological sample and functionalize the nucleic acids within the biological sample to permit embedding within a permeable matrix, such as a gel. The methods can then implement probe-based spatial analysis within the permeable matrix using a second set of probes designed to hybridize with both a target nucleic acid and with the first probes bound to the target proteins. The resulting hybridized first and second probes are bound by a capture probe including a spatial barcode to provide spatial information for interactions between nucleic acids and target proteins within the sample.

Methods of determining the location and/or abundance of an interaction between a target protein and a target nucleic acid in a biological sample are described. Typically, the methods include the steps of: (a) contacting a biological sample including a plurality of nucleic acids with a plurality of first probes, wherein a first probe of the plurality of first probes includes: (i) a target protein-binding moiety and a first oligonucleotide including a second-probe docking sequence and, optionally, a target protein identification barcode, and (ii) one or more functional group(s), under conditions suitable for the target protein-binding moiety to bind to a target protein; (b) modifying a 3′ and/or 5′ end of the plurality of nucleic acids in the biological sample with one or more functional group(s), whereby the target nucleic acid is present within the plurality of nucleic acids; (c) embedding the biological sample in a gel, including forming an interaction between the one or more functional group(s) and the gel; (d) hybridizing a plurality of second probes to the plurality of nucleic acids to form a plurality of hybridized second probes, where a hybridized second probe of the plurality of hybridized second probes hybridizes to the target nucleic acid and includes: (i) a first-probe docking sequence; (ii) a region complementary to the target nucleic acid; and (iii) a capture domain binding sequence; (e) combining the first probe and the hybridized second probe to form a combined probe including the capture domain binding sequence; (f) releasing the combined probe; (g) hybridizing the capture domain binding sequence of the combined probe to a capture domain of a capture probe including: (i) a spatial barcode and (ii) the capture domain; and (h) determining: (hi) the spatial barcode sequence or a complement thereof; (hii) all or a portion of the hybridized second probe sequence or a complement thereof; and/or (hiii) the sequence of the target protein identification barcode; and (i) using the determined sequences of (hi), (hii) and optionally (hiii) to identify the location and/or abundance of the interaction between the target protein and nucleic acid in the biological sample. Typically, the gel is a hydrogel. In some forms, the nucleic acid is DNA. In other forms, the nucleic acid is an RNA, such as small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (incRNA), or transfer RNA (tRNA). In preferred forms, the RNA is mRNA.

In some forms, the methods further include one or more steps of fragmenting the plurality of nucleic acids. In some forms, the plurality of nucleic acids is fragmented prior to step (a). In other forms, the fragmenting occurs after or during any one of steps (a), (b), (c), (d) or (e). In an exemplary method, the fragmenting includes contacting the biological sample with one or more nuclease enzymes. In some forms, the methods further include permeabilizing the biological sample. In some forms, the permeabilizing is performed during step (c), and/or during step (f). In an exemplary method, the permeabilizing includes contacting the biological sample with a permeabilization reagent. In some forms, the gel includes the permeabilization reagent. Exemplary permeabilizing reagents include an organic solvent, a detergent, and an enzyme, or a combination thereof. In some forms, the permeabilization agent is selected from an endopeptidase, a protease, sodium dodecyl sulfate (SDS), polyethylene glycol tert-octylphenyl ether, polysorbate 80, polysorbate 20, N-lauroylsarcosine sodium salt solution, and saponin. In other forms, the permeabilization agent includes a protease.

In some forms, step (f) and/or step (g) further includes degrading or otherwise removing the target nucleic acid hybridized to the combined probe and/or releasing the combined probe from the functional group(s). In some forms, the degrading or otherwise removing includes contacting the biological sample with a nuclease, such as a DNase or an RNase. In an exemplary method, the target nucleic acid is RNA and the RNase is an RNase H.

In some forms, the first oligonucleotide includes the target protein identification barcode. In some forms, the region complementary to the target nucleic acid includes a random sequence, for example, a sequence having from about 4 to about 10 nucleotides, inclusive. In some forms, the plurality of second probes includes a library of sequences complementary to all or part of each nucleic acid of the plurality of nucleic acids.

In some forms, the first probe docking sequence is substantially complementary to the second-probe docking sequence, and combining the first probe and the hybridized second probe includes hybridizing the first-probe docking sequence with the second-probe docking sequence. In other forms, the first-probe docking sequence is not complementary to the second-probe docking sequence, and combining the first probe and the hybridized second probe to form a combined probe including a capture domain binding sequence includes the use of a first splint oligonucleotide. In some forms, the first splint oligonucleotide includes (i) a sequence complementary to the first-probe docking sequence; and (ii) a sequence complementary to the second-probe docking sequence. In some forms, combining the first probe and the hybridized second probe includes hybridizing the first splint oligonucleotide to both the first-probe docking sequence and the second-probe docking sequence, and ligating the first probe and second hybridized probe together to form the combined probe. In some forms, the ligating includes ligating the second-probe docking sequence of the first probe and the first-probe docking sequence of the second probe together. In some forms, the ligating is chemical ligation or enzymatic ligation. In some forms, the first splint oligonucleotide includes one or more additional nucleic acid residues between the sequence complementary to the first-probe docking sequence and the sequence complementary to the second-probe docking sequence. Typically, the size and/or sequence of the first-probe docking sequence, and/or second-probe docking sequence, and/or the first splint oligonucleotide are configured so that combining the first probe and hybridized second probe will preferably occur when the target protein is bound directly to the target nucleic acid. For example, in some forms, the size and/or sequence of the first splint oligonucleotide is configured so that combining the first probe and hybridized second probe will preferably occur when the target protein interacts directly with the target nucleic acid. An exemplary size of the first splint oligonucleotide is between about 6 and about 100 nucleotides. An exemplary distance between the target protein and target nucleic acid is less than 0.8 nm, such as 0.1 nm, 0.2 nm, 0.3 nm, 0.35 nm, 0.4 nm, 0.45 nm and 0.5 nm. An exemplary size of the first oligonucleotide is between about 6 and about 100 nucleotides, inclusive. An exemplary size of the second probe is between about 6 and about 100 nucleotides, inclusive.

In some forms, the hybridized second probe includes a first and a second RNA-Templated Ligation (RTL) oligonucleotide, wherein each of the first and second RTL oligonucleotides include a sequence complementary to the target nucleic acid. In some forms, hybridizing a plurality of second probes to the plurality of nucleic acids includes (i) hybridizing the first RTL probe with the target nucleic acid; (ii) hybridizing a second RTL probe with the target nucleic acid; and (iii) ligating the first and second RTL probes together to form the hybridized second probe.

An exemplary target protein-binding moiety is selected from an antibody or antigen-binding fragment thereof, an aptamer, a lectin, a small molecule, an enzyme, a nucleic acid and a target molecule-specific ligand. An exemplary target protein binding moiety includes a nucleic acid. An exemplary target protein binding moiety includes an aptamer, such as an RNA aptamer or DNA aptamer.

In some forms, the sequence of the target protein binding moiety includes a target protein identification barcode. An exemplary target protein binding moiety includes a polypeptide, such as an immunoglobulin, or antigen-binding fragment thereof. An exemplary immunoglobulin is a monoclonal antibody, or a polyclonal antibody.

In some forms, the target protein-binding moiety is associated with the first oligonucleotide via a first cleavable linker, such as a photocleavable linker, UV-cleavable linker, or an enzyme-cleavable linker. An exemplary first cleavable linker includes a single stranded or double stranded nucleic acid, e.g., including a recognition sequence for one or more restriction enzymes. In an exemplary method, the target protein-binding moiety is associated with the first oligonucleotide via a first cleavable linker and the one or more functional groups (e.g., for gel embedding) is associated with the first probe via a second cleavable linker, and optionally whereby the first and second cleavable linkers are cleaved by different mechanisms.

In some forms, the method further includes, prior to modifying a 3′ and/or 5′ end of the plurality of nucleic acids in step (b), fragmenting the plurality of nucleic acids in the biological sample. In some forms, one or more functional group(s) is associated with the target protein-binding moiety, and/or the first oligonucleotide (e.g., 5′ end of the first oligonucleotide and/or 3′ end of the first oligonucleotide).

In some forms, the functional group(s) include one species of functional group. In other forms, the functional group(s) include more than one species of functional group. An exemplary functional group) includes acrydite.

Typically, the methods include determining (hi) the spatial barcode sequence or a complement thereof; and (hii) all or a portion of the second hybridized probe sequence or the complement thereof; and (hiii) the sequence of the target protein identification barcode. In some forms, the methods include using the determined sequences of (hi) and (hii) and (hiii). In some forms, the methods include, after step (a), substantially removing unbound first probe from the biological sample.

In some forms, the region complementary to the target nucleic acid is complementary to a poly(A) sequence, or a complement thereof. In some forms, the region of the second probe complementary to the target nucleic acid is complementary to a coding region of an mRNA, or a complement thereof. Exemplary target proteins are selected from Argonaute-1 (AGO-1), Argonaute-2 (AGO-2), Argonaute-3 (AGO-3), Argonaute-4 (AGO-4), P-element-induced wimpy testis (PIWI), Dicer, glycine/tryptophan (GW) repeats-containing 182 protein, heat shock protein 70 (Hsp70), and heat shock protein 90 (Hsp90). Exemplary target proteins include proteins that bind directly to and/or interact directly with one or more protein selected from Argonaute-1 (AGO-1), Argonaute-2 (AGO-2), Argonaute-3 (AGO-3), Argonaute-4 (AGO-4), P-element-induced wimpy testis (PIWI), Dicer, glycine/tryptophan (GW) repeats-containing 182 protein, heat shock protein 70 (Hsp70), and heat shock protein 90 (Hsp90). Exemplary target proteins are selected from Hu-antigen R (HuR), heterogeneous nuclear ribonucleoprotein family (hnRNP), the arginine/serine-rich splicing factor protein family (SRSF), and RNA-binding motif (RBM) proteins. Exemplary target proteins include a protein that binds directly to and/or interacts directly with one or more protein selected from Hu-antigen R (HuR), heterogeneous nuclear ribonucleoprotein family (hnRNP), the arginine/serine-rich splicing factor protein family (SRSF), and RNA-binding motif (RBM) proteins. In some forms, the target protein includes a transcription factor or an enhancer binding protein. In some forms, the target protein-binding moiety includes a plurality of target protein-binding moieties specific for a plurality of target proteins.

In some forms, the first probe further includes one or more functional domains. In some forms, a second probe of the plurality of second probes further includes one or more functional domains. In some forms, the first splint oligonucleotide further includes one or more functional domains. In some forms, the capture probe further includes one or more functional domains. Exemplary functional domains include a Unique Molecular Identified (UMI), or a primer binding site, or a label, or dye. In some forms, the region of the second probe complementary to one or more nucleic acids includes between about 4 and about 70 contiguous nucleotides, inclusive, optionally between about 8 and about 50 contiguous nucleotides, inclusive. In some forms, the second probe hybridizes to the nucleic acid in a region beginning 0-200 nucleotides, or any subrange thereof, relative to the end of a nucleic acid binding domain of the target protein. In some forms, the method further includes extending the hybridized second probe using the target nucleic acid as a template.

In some forms, the capture probe is included in an array including a plurality of capture probes. In some forms, the capture probe and/or the combined probe include one or more binding sites for sequencing primers. In some forms, the capture probe is associated with a substrate via a third linker. In some forms, the capture probe is associated with a gel or a bead. For example, in some forms, the capture probe is associated with the gel or the bead via a third cleavable linker. In some forms, the third cleavable linker is a photocleavable linker, UV-cleavable linker, or an enzyme-cleavable linker. In some forms, the ligating step includes enzymatic ligation or chemical ligation. For example, in some forms, the ligating step includes T4 ligase-mediated ligation. In some forms, the methods further include, prior to step (f), releasing the target nucleic acid from the combined capture probe. In some forms, the releasing includes enzymic digestion of the target nucleic acid. In some forms, the methods include, prior to step (g) or (h), separating the combined probe from the target protein-binding moiety of the first probe. In some forms, the separating includes cleavage of a first cleavable linker. In some forms, the methods include, prior to step (g) or (h), releasing the combined probe from the gel. In some forms, the releasing includes cleavage of a second cleavable linker. In some forms, the methods further include extending the combined probe using the capture probe as a template to generate an extended combined probe. In some forms, the methods further include extending the capture probe using the combined probe as a template to generate an extended capture probe. In some forms, the determining in step (h) includes amplifying all or part of the extended combined probe, or all or part of the extended capture probe, thereby generating an amplified product, i.e., when the amplified product includes all or part of the sequence of the combined probe or a complement thereof, and the sequence of the spatial barcode, or a complement thereof. In some forms, the determining in step (h) includes sequencing the amplified product.

In some forms, the target protein-binding moiety includes one or more functional domains. Exemplary functional domains include a Unique Molecular Identified (UMI), or a primer site, or a label, or dye. In some forms, embedding the biological sample in a gel further includes forming an interaction between the one or more functional group(s) in the plurality of nucleic acids and the gel.

In some forms, where the target nucleic acid is RNA, optionally mRNA, the one or more functional group(s) includes a 5′-phosphate group or a 5′-phosphate group modified with a leaving group. In some forms, the method includes contacting the biological sample with an attachment agent, whereby the attachment agent includes (i) at least one reactive moiety capable of reacting with at least one 5′-phosphate group of the RNA or 5′-phosphate group of the RNA modified with a leaving group, and (ii) at least one attachment moiety capable of attaching covalently or noncovalently to a matrix-forming agent; forming a covalent bond between the reactive moiety of the attachment agent and the RNA; contacting the biological sample with a matrix forming agent; and forming a three-dimensional polymerized matrix (e.g., gel) from the matrix-forming agent, thereby embedding the biological sample and immobilizing the RNA in the three-dimensional polymerized matrix/gel. In some forms, the method further includes reacting at least one RNA in the biological sample with a polynucleotide kinase to provide an RNA including a 5′-phosphate group, and optionally modifying the 5′-phosphate group with a leaving group to provide an RNA including a 5′-phosphate group modified with a leaving group. In some forms, the polynucleotide kinase is a T4 Polynucleotide Kinase (T4 PNK), or a T7 Polynucleotide Kinase (T7 PNK).

In some forms, the attachment agent is a compound of Formula (I):

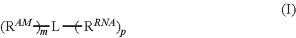

or a salt thereof, whereby each R^{RNA} is, independently, a reactive moiety capable of reacting with at least one 5′-phosphate group of the RNA or 5′-phosphate group of the RNA modified with a leaving group; each R^{AM} is, independently, an attachment moiety capable of attaching covalently or noncovalently to a matrix-forming agent; L is a bond or a linker moiety; m is an integer of from 1 to 4, inclusive; and p is an integer of from 1 to 4, inclusive. In some forms, at least one reactive moiety of the attachment agent includes or is a nucleic acid oligonucleotide having from 2 to 20 nucleotide residues, inclusive. In some forms, the nucleic acid oligonucleotide is a DNA oligonucleotide. In some forms, the method includes forming a covalent bond between a 3′-OH of the DNA oligonucleotide and the 5′-phosphate group of the RNA under catalysis of a ligase. In some forms, the ligase is T4 RNA Ligase 1.

In some forms, the method includes forming a covalent bond between the reactive moiety of the attachment agent and the 5′-phosphate group of the RNA without catalysis of an enzyme. The reactive moiety can include or is a nucleophilic group. For example, in some forms, the reactive moiety includes or is —OH, or, —SH, or —NH₂.

In some forms, the attachment agent is at least one attachment moiety capable of attaching covalently to a matrix-forming agent. An exemplary attachment moiety is or includes an alkenyl, alkynyl, allyl or vinyl moiety, ally ester moiety, an acrylamide moiety, an amide moiety, an alcohol moiety, a polyol moiety, a furan moiety, a maleimide moiety, a norbornene moiety, a thiol moiety, a sulfide moiety, a phenol moiety, a urethane moiety, a cyano moiety, an amino moiety, an isocyanate moiety, an isothiocyanate moiety, an ether moiety, a dextran moiety, or an alginate moiety.

In some forms, at least one attachment moiety is or includes a click functional group, optionally whereby at least one attachment moiety is or includes an azide moiety.

In some forms, when the target nucleic acid is RNA (e.g., mRNA) the RNA is fragmented and includes a 2′,3′-vicinal diol. In such forms, the method can further include contacting the biological sample with a formylation reagent, where the formylation reagent converts the 2′,3′-vicinal diol moiety into 2′3′-dialdehyde moiety, thereby forming the one or more functional group(s).

In some forms, the method further includes: (i) contacting the biological sample with an attachment agent including at least one aldehyde-reactive group capable of reacting with at least one aldehyde of the 2′,3′-dialdehyde moiety of the fragmented ribonucleic acid to form a covalent bond and at least one attachment moiety capable of reacting with a matrix-forming agent to form a covalent bond; (ii) contacting the biological sample with a matrix-forming agent; and (iii) forming a three-dimensional polymerized matrix from the matrix-forming agent, thereby embedding the biological sample in the three-dimensional polymerized matrix and anchoring the fragmented RNA to the three-dimensional polymerized matrix.

In some forms, the method can further include contacting the biological sample including the fragmented RNA with a 3′ phosphatase to provide the fragmented RNA including a 2′,3′-vicinal diol. In some forms, where the fragmented RNA has a 2′,3′cyclo-phosphate fragmentation at 3′-terminal end or a 2′ hydroxyl and a 3′ phosphate fragmentation at 3′-terminal end, a 3′ phosphatase catalyzes the formation of the 2′,3′-vicinal diol. In some forms, the 3′ phosphatase is T4 polynucleotide kinase.

In some forms, the attachment agent is N-(2-aminoethyl) methacrylamide, 2-aminoethyl methacrylate, or 2-aminoethyl (E)-but-2-enoate. In some forms, the method further includes contacting the biological sample with a reducing agent, such as, but not limited to, sodium borohydride.

In some forms, the matrix-forming agent is or includes an agent selected from acrylamide, bisacrylamide, cellulose, alginate, polyamide, agarose, dextran, or polyethylene glycol, or a combination thereof. In some forms, the three-dimensional polymerized matrix is formed by subjecting the matrix-forming agent to polymerization. In some forms, the polymerization is initiated by adding a polymerization-inducing catalyst, exposing UV light or functional cross-linkers to the biological sample. Releasable nucleic acid probes are also provided. Typically, the probes include: (i) a target protein-binding moiety and an associated first oligonucleotide including a non-embeddable second-probe docking sequence and, optionally, a target protein identification barcode, and (ii) one or more functional group(s) for gel embedding associated with the target protein-binding moiety or the end of the first oligonucleotide proximal thereto. In some forms, the target protein-binding moiety is associated with the first oligonucleotide via a first cleavable linker, such as a photocleavable linker, UV-cleavable linker, or an enzyme-cleavable linker. In some forms, the target protein-binding moiety is associated with the first oligonucleotide via a first cleavable linker, wherein the first cleavable linker includes single stranded or double stranded nucleic acid. For example, in some forms, the single stranded or double stranded nucleic acid includes a recognition and/or cut sequence for one or more restriction enzymes. An exemplary method of determining a target protein interaction with a target nucleic acid includes (a) contacting a biological sample including a plurality of nucleic acids with a plurality of first probes, wherein a first probe of the plurality of first probes includes (i) a target protein-binding moiety and an associated first oligonucleotide including a second-probe docking sequence and, optionally, a target protein identification barcode, and (ii) one or more functional group(s), under conditions suitable for the target protein-binding moiety to bind to a target protein; (b) modifying a 3′ and/or 5′ end of the plurality of nucleic acids in the biological sample with one or more functional group(s); (c) embedding the biological sample in a gel, including forming an interaction between the one or more functional group(s) and the gel; (d) hybridizing a plurality of second probes to the plurality of nucleic acids to form a plurality of hybridized second probes, wherein a hybridized second probe of the plurality of hybridized second probes includes (i) a first-probe docking sequence; and (ii) a region complementary to a target nucleic acid within the plurality of nucleic acids; (e) combining the first probe and the hybridized second probe to form a combined probe including a capture domain binding sequence; (f) hybridizing the capture domain binding sequence of the combined probe to the capture domain of a capture probe including (i) a spatial barcode; (ii) a capture domain; and (g) determining: (gi) the spatial barcode sequence or a complement thereof; (gii) all or a portion of the hybridized second probe sequence or a complement thereof; and/or (giii) the sequence of the target protein identification barcode.

In some forms, the biological sample includes a tissue sample or tissue section. In some forms, the tissue sample or tissue section includes a Formalin-Fixed Paraffin-Embedded (FFPE) tissue sample or tissue section. In some forms, the tissue sample or tissue section includes a frozen and/or lyophilized tissue sample or tissue section. In some forms, the method further includes, prior to step (a) one or more steps of de-crosslinking the tissue sample. In some forms, method further includes, prior to step (a), deparaffinizing the tissue sample. In some forms, deparaffinizing includes contacting the tissue sample with a solvent.

In some forms, the described methods further include one or more steps of staining and/or labelling the biological sample. For example, in some forms, the one or more steps of staining and/or labelling the tissue sample is prior to step (a). In some forms, the one or more steps of staining and/or labelling the tissue sample is during or after step (a), or during or after step (b) or during step (c). In some forms, the staining the tissue sample includes hematoxylin and/or eosin (H and E) staining. In some forms, the methods further include imaging the stained and/or labelled tissue sample, optionally further including de-staining the tissue sample.

Kits of reagents for performing the described methods are also provided. Typically, the kits include (a) a plurality of first probes, wherein a first probe of the plurality of first probes includes: (i) a target protein-binding moiety and an associated first oligonucleotide including a non-embeddable second-probe docking sequence and, optionally, a target protein identification barcode and (ii) one or more functional group(s) for gel embedding; and (b) instructions for performing the described methods.

### BRIEF DESCRIPTION OF THE DRAWINGS

The following drawings illustrate certain forms of the features and advantages of this disclosure. These forms are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped (inferior) side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIGS. 12A-12D are schematic diagrams depicting an exemplary arrangement of molecules configured for enhanced spatial detection and analysis of interactions between nucleic acids (131040) and a “first” protein capture probe (131010). FIG. 12A depicts an exemplary protein capture probe having a nucleic acid capture agent (132001) coupled to the 5′ terminus of a first single-stranded oligonucleotide (131002) component via a linker (13100). FIG. 12B depicts an exemplary protein capture probe having a nucleic acid capture agent (132001) coupled directly to the 3′ terminus of a second single-stranded oligonucleotide (132003) via a linker (13100), and indirectly to the 5′ terminus of the first single-stranded oligonucleotide (131002) through its hybridization with the second oligonucleotide. FIG. 12C depicts an exemplary protein capture probe having a polypeptide capture agent (13116) coupled via a linker (13100) to the 5′ terminus of an oligonucleotide (131002) hybridized to a second oligonucleotide (132004) functionalized at the 3′ terminus. FIG. 12D depicts an exemplary protein capture probe having a polypeptide capture agent (13116) coupled via a linker (13100) directly to the 5′ terminus of an oligonucleotide (131002) hybridized to a second oligonucleotide (132004), which is hybridized to a third oligonucleotide (132112) functionalized at the 5′ terminus. FIG. 12E is a schematic diagram depicting a single bi-specific probe (131019), including the components of the first probe of any of FIGS. 12A-12D, as well as a nucleic acid-binding domain (13170) and a capture domain binding sequence (13180), as well as optionally a bridging or spanning domain (13139).

FIGS. 13A-13B are schematic diagrams depicting binding of a functionalized protein capture probe (131010) to a target nucleic acid binding protein (13200) (FIG. 13A); and functionalizing (★) nucleic acids (131040) in the sample (FIG. 13B).

FIGS. 14A-14B are schematic diagrams depicting embedding a sample containing the probes and nucleic acid within a permeable matrix(S), such as a gel (FIG. 14A) and permeabilization of the sample and removal of protein components (FIG. 14B).

FIGS. 15A-15E are schematic diagrams depicting contacting the permeabilized sample embedded within the matrix with an exemplary “second” nucleic acid capture probe having a nucleic acid capture agent (131020), and an optional splint oligonucleotide (131030) (FIG. 15A); ligation of a “first” target protein binding probe and the “second” nucleic-acid probe to form a combined probe (131240) (FIG. 15B). FIG. 15C depicts the binding of a target nucleic acid by a single, bi-specific probe, as depicted in FIG. 12E. FIG. 15D depicts isolation of the linear combined probe (131240) by removal of sample components; and FIG. 15E depicts nuclease treatment of the combined probe to remove bound nucleic acids (131250).

FIGS. 16A-16B are schematic diagrams depicting capture of a combined probe (131250) by a capture probe bound to a substrate (131700) to form a captured probe (131070) (FIG. 16A); and splitting from the protein capture domain (132001) and of the substrate to provide a minimal captured probe (131071) amenable for sequence analysis (FIG. 16B).

FIG. 17 is a simplified flow chart showing a schematic overview of an exemplary form of the described methods for spatial profiling of RNA binding protein (RBP)/RNA interactions, including the steps of: Baking a tissue slide; Dewaxing; Rehydrating, H and E staining; BF imaging; Destaining; RBP detection with a tagged construct; Modification of RNA with a functional group for gel tethering; Tissue embedding; De-crosslinking tissue within the gel; Tissue Removal of clearing; RNA-specific Probe hybridization (e.g., RNA Templated Ligation; RTL); RBP probe-RTL ligation to form a combined probe; Release of the RNA from the RTL/combined probe (e.g., with RNase H); and Release of the combined probe within the hydrogel/hydrogel sandwich for capture on a capture probe associated with a spatial barcode/spatial array for probe extension, and sequencing/library prep. The exemplary workflow, which initiates with a standard FFPE sample, can be implemented for existing spatial analysis workflows, for example Visium Spatial Gene Expression molecular profiling for classifying tissue based on total mRNA.

### DETAILED DESCRIPTION

### I. Spatial Analysis

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2):e0212031, 2019; Chen et al., Science 348 (6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some forms, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some forms, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA). Additional examples of analytes can be found in Section (I)(c) of PCT Publication No. WO2020/176788 and/or U.S. Patent Application Publication No. 2020/0277663. In some forms, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some forms, the biological sample is a tissue sample. In some forms, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some forms, the biological sample is a tissue. In some forms, the tissue sample is a solid tissue sample. In some forms, the biological sample is a tissue section (e.g., a fixed tissue section). In some forms, the tissue is flash-frozen and sectioned. Any suitable method described herein or known in the art can be used to flash-freeze and section the tissue sample. In some forms, the biological sample, e.g., the tissue, is flash-frozen using liquid nitrogen before sectioning. In some forms, the biological sample, e.g., a tissue sample, is flash-frozen using nitrogen (e.g., liquid nitrogen), isopentane, or hexane.

In some forms, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some forms, the sectioning is performed by cryosectioning, for example using a microtome. In some forms, the methods further include a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some forms, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some forms, the biological sample, e.g., the tissue sample, is fixed in a fixative including alcohol, for example methanol. In some forms, instead of methanol, acetone, or an acetone-methanol mixture can be used. In some forms, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not de-crosslinked afterward. In some preferred forms, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not de-crosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some forms, fixation of the biological sample e.g., using acetone and/or alcohol (e.g., methanol, acetone-methanol) is performed while the sample is mounted on a substrate (e.g., glass slide, such as a positively charged glass slide).

In some forms, the biological sample, e.g., the tissue sample, is fixed e.g., immediately after being harvested from a subject. In such forms, the fixative is preferably an aldehyde fixative, such as paraformaldehyde (PFA) or formalin. In some forms, the fixative induces crosslinks within the biological sample. In some forms, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some forms when a fixed frozen tissue sample is treated with a sucrose gradient, it can be rehydrated with an ethanol gradient. In some forms, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred forms, a fixed frozen biological sample is not treated with methanol. In preferred forms, a fixed frozen biological sample is not paraffin embedded. Thus, in preferred forms, a fixed frozen biological sample is not deparaffinized. In some forms, a fixed frozen biological sample is rehydrated in an ethanol gradient.

In some instances, the biological sample (e.g., a fixed frozen tissue sample) is treated with a citrate buffer. Citrate buffer can be used for antigen retrieval to de-crosslink antigens and fixation medium in the biological sample. Thus, any suitable decrosslinking agent can be used in addition to or alternatively to citrate buffer. In some forms, for example, the biological sample (e.g., a fixed frozen tissue sample) is de-crosslinked with TE buffer.

In any of the foregoing, the biological sample can further be stained, imaged, and/or destained. For example, in some forms, a fresh frozen tissue sample or fixed frozen tissue sample is stained (e.g., via eosin and/or hematoxylin), imaged, destained (e.g., via HCl), or a combination thereof. In some forms, when a fresh frozen tissue sample is fixed in methanol, it is treated with isopropanol prior to being stained (e.g., via eosin and/or hematoxylin), imaged, destained (e.g., via HCl), or a combination thereof. In some forms when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some forms, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One.; 6 (11):e27704 (2011); and Mathieson W. et al., Am J Clin Pathol.; 146 (1): 25-40 (2016), each of which are hereby incorporated by reference in their entirety, for a description and evaluation of PAXgene for tissue fixation. Thus, in some forms, when the biological sample, e.g., the tissue sample, is fixed in a fixative including alcohol, the fixative is PAXgene. In some forms, a fresh frozen tissue sample is fixed with PAXgene. In some forms, a fixed frozen tissue sample is fixed with PAXgene.

In some forms, the biological sample, e.g., the tissue sample is fixed, for example in methanol, acetone, acetone-methanol, PFA, PAXgene or is formalin-fixed and paraffin-embedded (FFPE). In some forms, the biological sample includes intact cells. In some forms, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some forms, the imaging occurs prior to destaining the sample. In some forms, the biological sample is stained using an H&E staining method. In some forms, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some forms, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some forms, the tissue sample is derived from normal or diseased tissue. In some forms, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following forms can be used with any of the methods described herein. In some forms, the biological sample (e.g., a fixed and/or stained biological sample) is imaged. In some forms, the biological sample is visualized or imaged using bright field microscopy. In some forms, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some forms, the sample is stained and imaged prior to adding reagents for analyzing captured analytes the primer to the biological sample.

In some forms, the methods include staining the biological sample. In some forms, the staining includes the use of hematoxylin and/or eosin. Non-limiting examples of stains include histological stains (e.g., hematoxylin and/or eosin) and immunological stains (e.g., fluorescent stains). In some forms, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some forms, the staining includes the use of a detectable label selected from the group including a radioisotope, a fluorophore, a chemiluminescent compound, a bioluminescent compound, or a combination thereof.

In some forms, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Forms Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some forms, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some forms, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some forms, the endopeptidase is pepsin. In some forms, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some forms, the capture probe is a nucleic acid or a polypeptide. In some forms, the capture probe includes a barcode (e.g., a spatial barcode and/or a unique molecular identifier (UMI)) and a capture domain). In some instances, the capture probe includes a homopolymer sequence, such as a poly(T) sequence. In some forms, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other, and can be interrupted by one or more non-complementary residues within the complementary nucleic acid sequence. In some forms, at least 60%, but less than 100%, of the residues of one of the two complementary nucleic acid sequences are complementary to residues in the other nucleic acid sequence. In some forms, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some forms, the biological sample is mounted on a first substrate and the substrate including the array of capture probes is a second substrate. During this process, one or more analytes or analyte derivatives (e.g., intermediate agents; e.g., ligation products) are released from the biological sample and migrate or transfer to the second substrate including an array of capture probes. In some forms, the release and migration/transfer of the analytes or analyte derivatives to the second substrate including the array of capture probes occurs in a manner that preserves the original spatial context of the analyte or analyte derivative in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

Prior to transferring analytes from the biological sample to the array of features (e.g., containing capture probes) on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

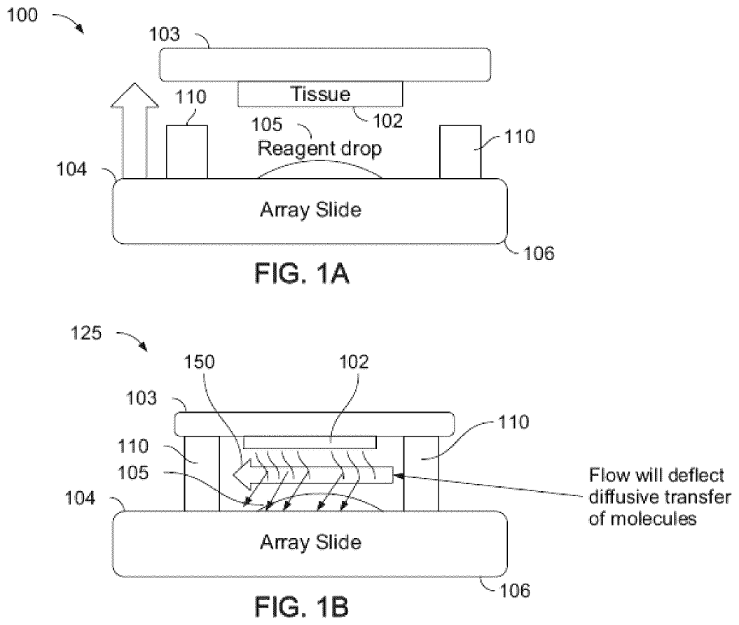

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some forms, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some forms wherein the biological sample 102 has been pre-permeabilized, the reagent medium is not a permeabilization solution. Herein, the reagent medium may also include one or more of a monovalent salt, a divalent salt, ethylene carbonate, and/or glycerol. In some forms, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain forms, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some forms, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some forms, the separation distance is less than 50 microns. In some forms, the separation distance is less than 25 microns. In some forms, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some forms of a sample holder, the sample holder can include a first member including a first retaining mechanism configured to retain a first substrate including a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some forms, the adjustment mechanism includes a linear actuator. In some forms, the linear actuator is configured to move the second member along an axis orthogonal to the plane of the first member and/or the second member. In some forms, the linear actuator is configured to move the first member along an axis orthogonal to the plane of the first member and/or the second member. In some forms, the linear actuator is configured to move the first member, the second member, or both the first member and the second member at a velocity of at least 0.1 mm/sec. In some forms, the linear actuator is configured to move the first member, the second member, or both the first member and the second member with an amount of force of at least 0.1 lbs.

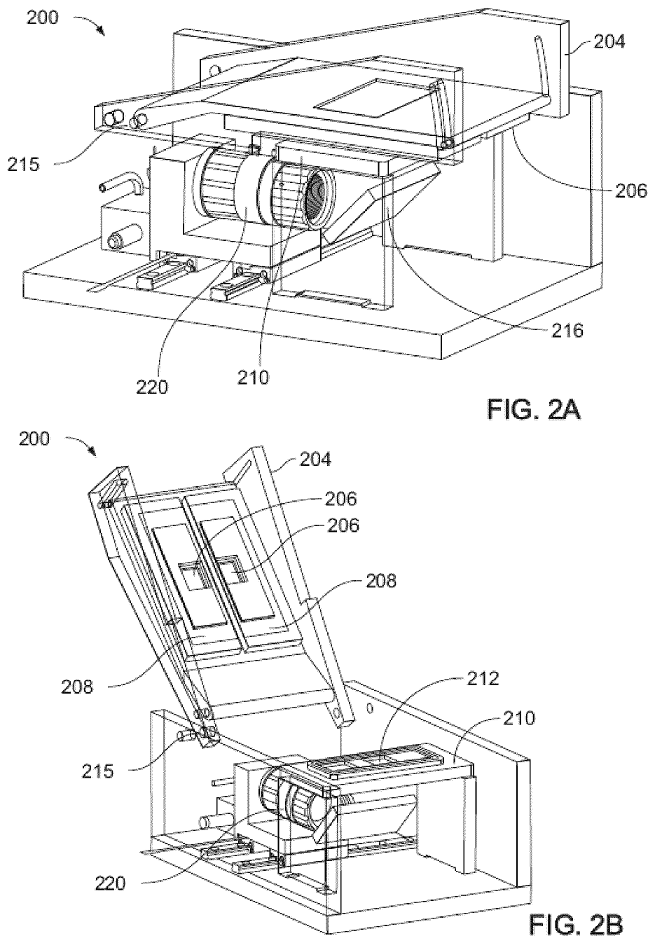

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some forms, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some forms, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

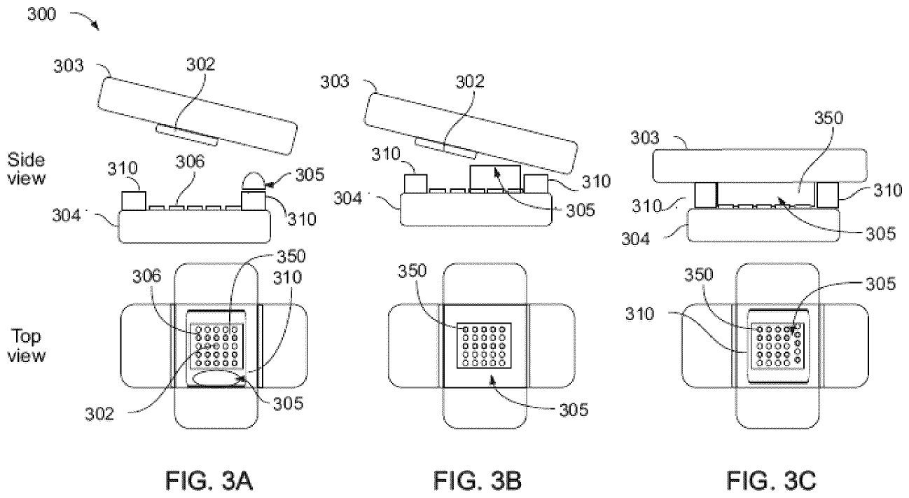

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer. FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed. In some forms, the first substrate and/or the second substrate are further moved to achieve an approximately parallel arrangement of the first substrate and the second substrate. FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305. While FIG. 3C depicts the first substrate (e.g., the slide 303 including biological sample 302) angled over (superior to) the second substrate (e.g., slide 304) and the second substrate including the spacer 310, it should be understood that an exemplary angled closure workflow can include the second substrate angled over (superior to) the first substrate and the first substrate including the spacer 310. It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

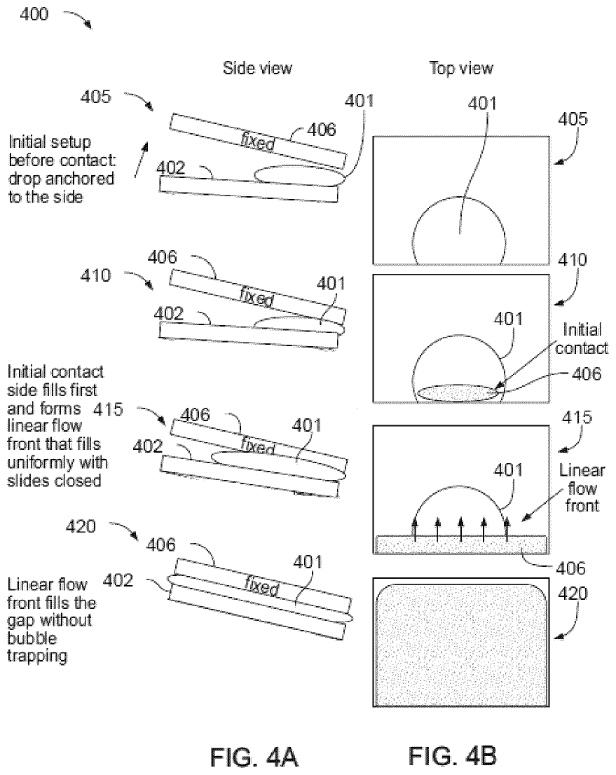

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402 contacting the spring.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates. At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some forms, the reagent medium (e.g., 105 in FIG. 1A) includes a permeabilization agent. In some forms, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some forms, the detergent is an anionic detergent (e.g., SDS or N-lauroylsarcosine sodium salt solution).

In some forms, the reagent medium includes a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some forms, the reagent medium includes a protease. Exemplary proteases include, e.g., pepsin, trypsin, pepsin, elastase, and proteinase K. In some forms, the reagent medium includes a nuclease. In some forms, the nuclease includes an RNase. In some forms, the RNase is selected from RNase A, RNase C, RNase H, and RNase I. In some forms, the reagent medium includes one or more of sodium dodecyl sulfate (SDS) or a sodium salt thereof, proteinase K, pepsin, N-lauroylsarcosine, and RNase.

In some forms, the reagent medium includes polyethylene glycol (PEG). In some forms, the PEG is from about PEG 2K to about PEG 16K. In some forms, the PEG is PEG 2K, 3K, 4K, 5K, 6K, 7K, 8K, 9K, 10K, 11K, 12K, 13K, 14K, 15K, or 16K. In some forms, the PEG is present at a concentration from about 2% to 25%, from about 4% to about 23%, from about 6% to about 21%, or from about 8% to about 20% (v/v).

In certain forms a dried permeabilization reagent is applied or formed as a layer on the first substrate or the second substrate or both prior to contacting or bringing into proximity the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some forms, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some forms, extending the capture probe includes adding to a 3′ end of a capture probe a nucleic acid sequence that is complementary to a nucleic acid sequence of an analyte or intermediate agent specifically bound to the capture domain of the capture probe. In some forms, the capture probe is extended by a reverse transcriptase. In some forms, the capture probe is extended using one or more DNA polymerases. In some forms, the extended capture probes include the sequence of the capture domain, the sequence of the spatial barcode of the capture probe and the complementary sequence of the template used for extension of the capture probe.

In some forms, extended capture probes are amplified (e.g., in bulk solution or on the array) to yield quantities that are sufficient for downstream analysis, e.g., sequencing. In some forms, extended capture probes (e.g., DNA molecules) can act as templates for an amplification reaction (e.g., a polymerase chain reaction).

Additional variants of spatial analysis methods, including in some forms, an imaging step, are described in Section (II)(a) of PCT Publication No. WO2020/176788 and/or U.S. Patent Application Publication No. 2020/0277663. Analysis of captured analytes (and/or intermediate agents or portions thereof), for example, including sample removal, extension of capture probes using the captured analyte or a proxy thereof as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663. Some quality control measures are described in Section (II)(h) of PCT Publication No. WO2020/176788 and/or U.S. Patent Application Publication No. 2020/0277663.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some forms, some or all of the features in an array are functionalized for analyte capture. Exemplary substrates are described in Section (II)(c) of PCT Publication No. WO2020/176788 and/or U.S. Patent Application Publication No. 2020/0277663. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) including capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application

### Publication No. 2020/0277663.

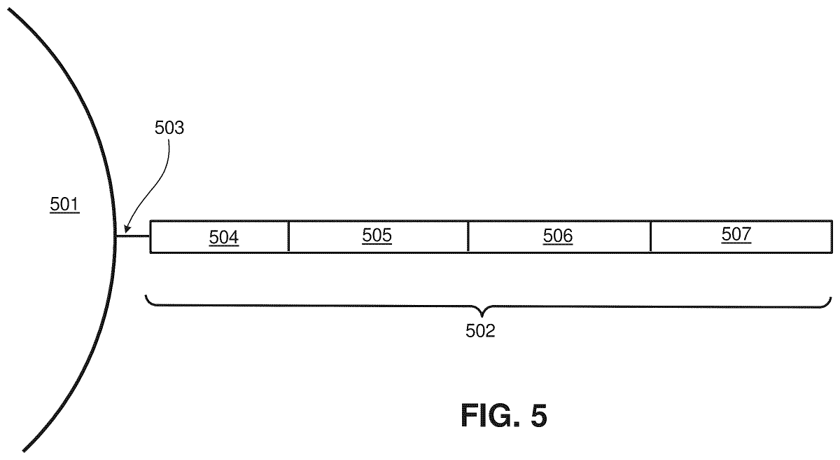

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence (or analyte capture sequence) present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

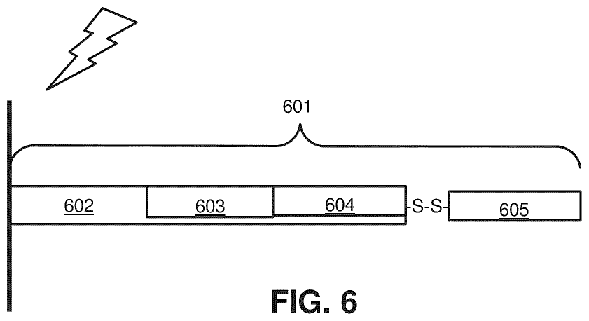

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

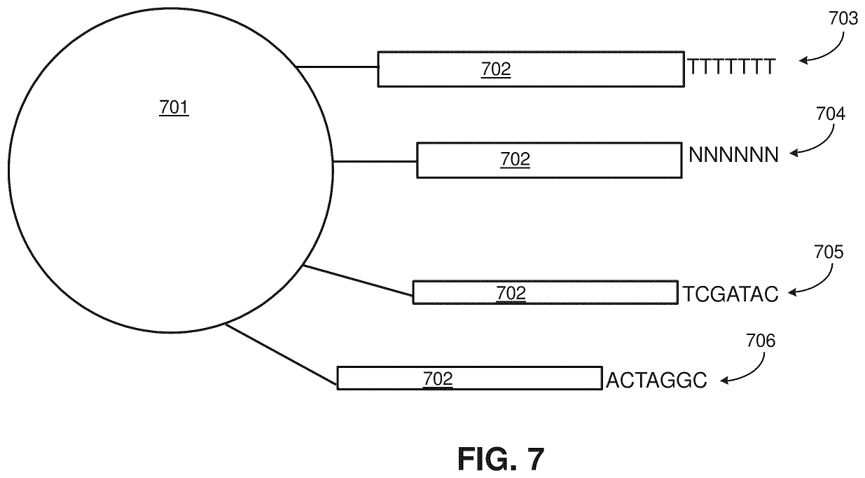

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some forms, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some forms, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some forms, functional sequences can be selected for compatibility with other sequencing systems, including non-commercialized sequencing systems.

In some forms, the spatial barcode 505 and functional sequences 504 are common to all of the probes attached to a given feature. In some forms, the UMI sequence 506 of a capture probe attached to a given feature is different from the UMI sequence of a different capture probe attached to the given feature.

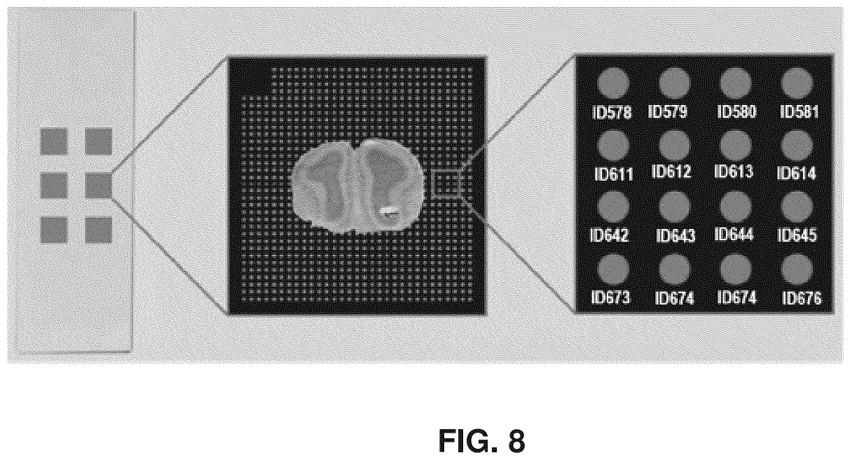

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some forms, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some forms, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some forms, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids, (e.g., mRNA). Thus, in some forms, the disclosed methods can include contacting the biological sample with a deoxyribonuclease (DNase). The DNase can be an endonuclease or exonuclease. In some forms, the DNase digests single- and/or double-stranded DNA. Suitable DNases include, without limitation, a DNase I and a DNase II. Use of a DNase as described can mitigate false positive sequencing data from off target gDNA ligation events.

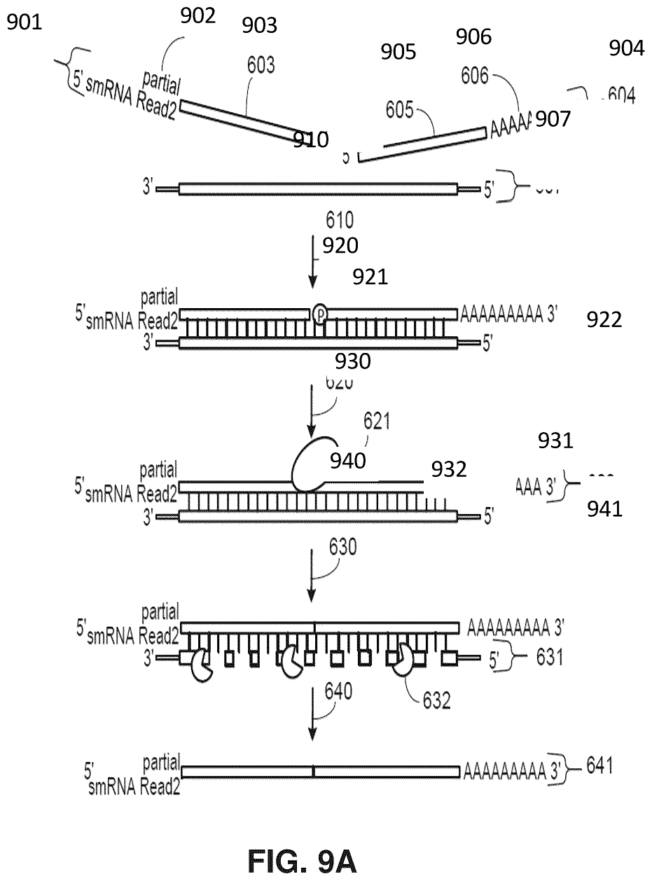

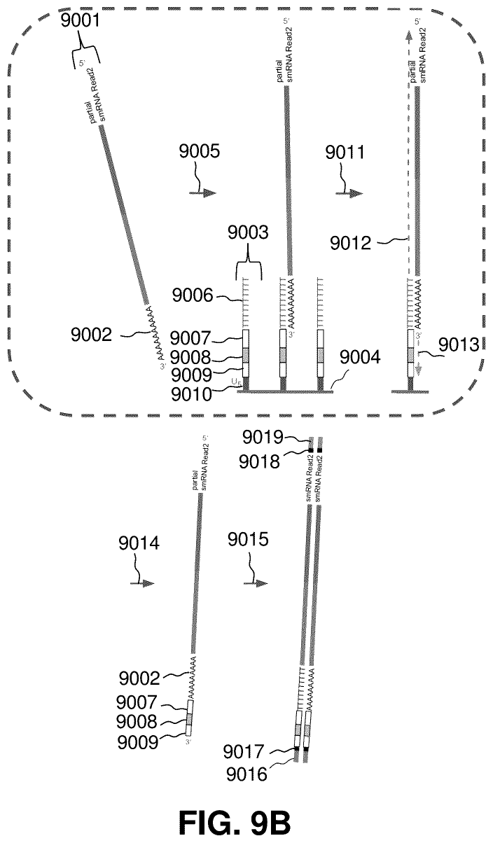

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some forms, as shown in FIG. 9B, the ligation product 9001 includes a capture probe capture domain 9002, which can bind to a capture probe 9003 (e.g., a capture probe immobilized, directly or indirectly, on a substrate 9004). In some forms, methods provided herein include contacting 9005 a biological sample with a substrate 9004, wherein the capture probe 9003 is affixed to the substrate (e.g., immobilized to the substrate, directly or indirectly). In some forms, the capture probe capture domain 9002 of the ligated product specifically binds to the capture domain 9006. The capture probe can also include a unique molecular identifier (UMI) 9007, a spatial barcode 9008, a functional sequence 9009, and a cleavage domain 9010.

In some forms, methods provided herein include permeabilization of the biological sample such that the capture probe can more easily capture the ligation products (i.e., compared to no permeabilization). In some forms, reverse transcription (RT) reagents can be added to permeabilized biological samples. Incubation with the RT reagents can extend the capture probes 9011 to produce spatially-barcoded full-length cDNA 9012 and 9013 from the captured ligation products.

In some forms, the extended ligation products can be denatured 9014 from the capture probe and transferred (e.g., to a clean tube) for amplification, and/or library construction. The spatially-barcoded ligation products can be amplified 9015 via PCR prior to library construction. P5 9016, i5 9017, i7 9018, and P7 9019, and can be used as sample indexes. The amplicons can then be sequenced using paired-end sequencing using TruSeq Read 1 and TruSeq Read 2 as sequencing primer sites.

In some forms, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some forms, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

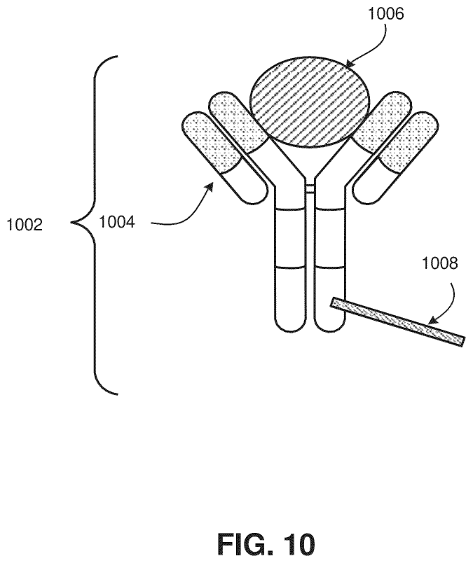

FIG. 10 is a schematic diagram of an exemplary analyte capture agent 1002 included of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008, which serves to identify the analyte binding moiety and a capture sequence which can hybridize to at least a portion or an entirety of a capture domain of a capture probe. The analyte-binding moiety 1004 can include a polypeptide and/or an aptamer. The analyte-binding moiety 1004 can include an antibody or antibody fragment (e.g., an antigen-binding fragment).

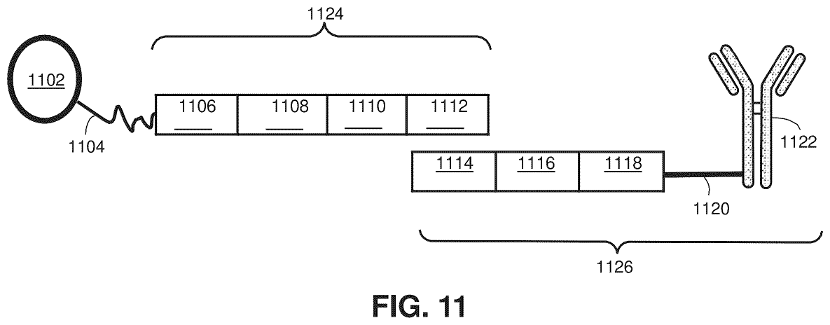

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and a UMI 1110, as described elsewhere herein. The capture probe can be affixed 1104 to a feature such as a bead 1102. The capture probe can also include a capture domain 1112 that is capable of binding to an analyte capture agent 1126. The analyte-binding moiety barcode domain of the analyte capture agent 1126 can include a functional sequence 1118, analyte binding moiety barcode 1116, and an analyte capture sequence 1114 that is capable of binding (e,g, hybridizing) to the capture domain 1112 of the capture probe 1124. The analyte capture agent can also include a linker 1120 that allows the analyte-binding moiety barcode domain (e.g., including the functional sequence 1118, analyte binding barcode 1116, and analyte capture sequence 1114) to couple to the analyte binding moiety 1122. In some forms, the linker is a cleavable linker. In some forms, the cleavable linker is a photo-cleavable linker, a UV-cleavable linker, or an enzyme cleavable linker. In some instances, the cleavable linker is a disulfide linker. A disulfide linker can be cleaved by use of a reducing agent, such as dithiothreitol (DTT), Beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some forms, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at pre-determined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Forms section of PCT Publication No. WO2020/176788 and/or U.S. Patent Application Publication No. 2020/0277663. See, for example, the Exemplary form starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some forms, spatial analysis can be performed using dedicated hardware and/or software, such as any of the systems described in Sections (II)(e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Enhanced Methods for Spatial Analysis of Interactions Between Nucleic Acid(s) and Nucleic Acid-Binding Analyte(s)

Methods for enhanced spatial analysis direct or indirect of interactions between a target nucleic acid binding analyte and a nucleic acid in a biological sample, such as a tissue sample, have been developed. The described methodologies and compositions for enhanced spatial analysis of nucleic acids interacting with target nucleic acid binding analytes can provide a vast amount of analyte and/or expression data for a variety of analytes within a biological sample, while retaining native spatial context. The methods target a nucleic acid binding analyte within a sample and identify nucleic acids that interact with the target nucleic acid binding analyte.

The terms “nucleic acid binding molecule”, “nucleic acid binding analyte” are used interchangeably herein. Exemplary nucleic acid binding molecules include, but are not limited to DNA binding proteins, RNA binding proteins, and complexes including two or more proteins of which at least one binds to or associates with a nucleic acid.

In some forms, the methods allow for identification of nucleic acid molecules that indirectly interact with a nucleic acid binding analyte, e.g., nucleic acid molecules that interact with a second nucleic acid binding analyte which interacts with the first targeted nucleic acid binding analyte. Exemplary target nucleic acid binding analytes are non-nucleic acids, such as polypeptides, proteins, lipids and carbohydrates. In some forms, a target nucleic acid binding analyte is a protein, such as a nucleic-acid binding protein.

The methods typically employ one or more first oligonucleotide probes having attached thereto a binding moiety that specifically binds to a target nucleic acid-binding analyte.

The term “specifically binds” refers to the binding of a first molecule to a physiological ligand molecule. An example of a specific binding interaction is binding of an antibody to its cognate antigen (such as a protein) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The first oligonucleotide probe selectively binds to the targeted nucleic acid-binding analyte within a biological sample, such as a tissue sample. The methods then employ one or more second oligonucleotide probes having attached thereto a binding moiety that specifically binds to a target nucleic acid.

The first and optionally the second probe generally include one or more functional moieties for embedding the probes within a permeable matrix, such as a gel. The one or more functional domains are typically located at one of a 5′ or 3′ terminus of each of the probes. The methods include one or more steps of further functionalizing the nucleic acids in the sample with one or more functional moieties for embedding the nucleic acids within the sample in the matrix. The methods then include embedding the probes and sample within the matrix, such as a gel. The embedding typically includes binding one or the other of a 5′ or 3′ terminus of each of the probes to a specific location within the matrix, such that one end of the probe is immobilized, while the other end may move within the gel.

Both the first and second oligonucleotide probes generally include complementary probe-binding motifs that are able, upon coming into contact with a complementary motif, hybridize to form a chimeric, or “combined” first and second probe, optionally with the assistance of a splinting oligonucleotide. The size, configuration and location of the functionalized motif embedded within the matrix for both the first and second probes is typically such that formation of the combined probe will only occur when the target nucleic acid and the target nucleic acid binding analyte to which they are respectively targeted interact with one another. For example, the location of the functionalized motif at one or the other of a 5′ or 3′ terminus of each of the probes can provide a degree of freedom of movement that permits the formation of a combined probe when both the target nucleic acid and the target nucleic binding analyte are within a suitable distance of one another. The methods then permeabilize the matrix and employ a capture probe including a spatial barcode and a combined probe capture domain to hybridize with the combined probe and determine spatial information for the interaction between the nucleic acid and the nucleic acid binding analyte within the sample.

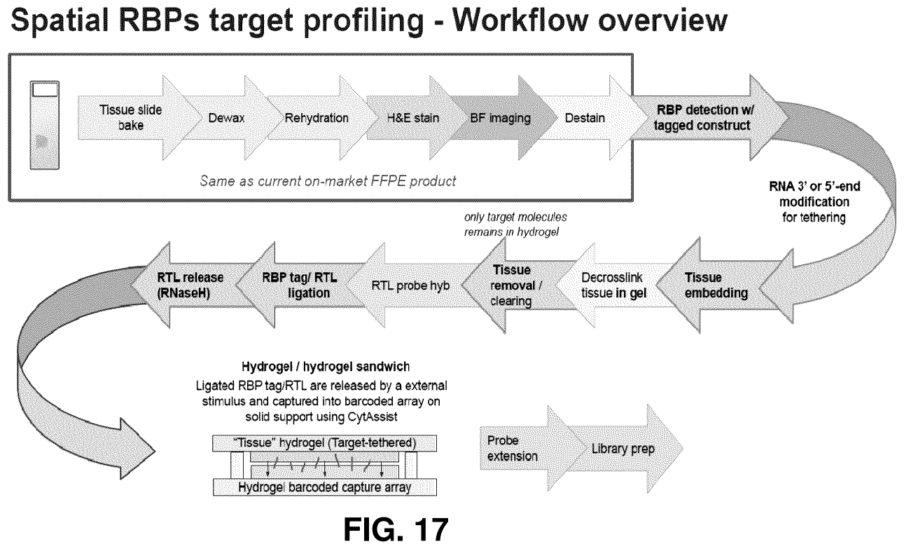

An exemplary methodology is depicted in the workflow set forth in the schematic of FIG. 17, including the preparatory steps (Baking a tissue slide; Dewaxing; Rehydrating, H and E staining; BF imaging; and Destaining), the RNA-binding protein/RNA interaction method steps (RBP detection with a tagged construct; Modification of RNA with a functional group for gel tethering; Tissue embedding; De-crosslinking tissue within the gel; Tissue Removal of clearing; RNA-specific Probe hybridization (e.g., RNA Templated Ligation; RTL); RBP probe-RTL ligation to form a combined probe; Release of the RNA from the RTL/combined probe (e.g., with RNAse H)), and standard probe capture and sequence analysis steps (Release of the combined probe within the hydrogel/hydrogel sandwich for capture on a capture probe associated with a spatial barcode/spatial array for probe extension, and sequencing/library prep).

The methods are described in greater detail, below.

A. Methods for Determining Location and/or Abundance of Interactions

Methods for determining a location and/or abundance of an interaction between a target nucleic acid-binding analyte, such as a protein, and a nucleic acid in a biological sample are provided. The interaction can be direct or indirect. For example, in some forms, the nucleic acid binding analyte is a protein that interacts with (e.g., binds to) a nucleic acid-binding protein (e.g., an RNA binding protein). In some forms, the nucleic acid-binding analyte is a nucleic acid-binding protein. For example, in some forms, the methods determine the presence, location and/or abundance of an interaction between a target nucleic-acid binding protein and a bound nucleic acid in a biological sample. Methods for detecting and measuring the presence and/or abundance of an interaction between a nucleic acid and a non-nucleic acid binding partner include using a first oligonucleotide probe (also referred to herein as a “first probe”) targeting a binding partner and functionalized to include a moiety for embedding within a matrix, such as a gel, and a second oligonucleotide probe (also referred to herein as a “second probe”) having specificity for a target nucleic acid, respectively. Both of the first and second probes include a probe interaction domain (also described as “docking sequence”) that is able, upon contacting a complementary probe interaction domain, of hybridizing to form a chimeric combined first and second probe. The enhanced methods characterize interactions between nucleic acids and non-nucleic acid analytes by selectively functionalizing and fixing the analytes in a permeable matrix that retains the native spatial information within the biological sample. The methods employ chemical modification of probes to “embed” target probes within a matrix at the site of target (i.e., protein and nucleic acid) recognition. The methods then permeabilize the matrix, for example, by exposure to proteases and/or acids to remove all non-nucleic acid components, followed by selective ligation between proximal probes and subsequent targeted capture of ligated probes using a capture probe associated with a spatial barcode to impart spatial information.

Typically, the described methods for proximity-based hybridization or ligation of a first probe including a target protein-binding moiety immobilized within a permeable matrix to a second probe and capture of the combined or ligated product (e.g., a chimeric probe that includes the first probe and the second probe hybridized or ligated together) is used to determine the location of at least one analyte and/or one interaction in a biological sample. The methods enhance the efficiency of target capture and identification compared to systems that use one oligonucleotide to associate with (e.g., hybridize to, attach to, or bind to) an analyte in a biological sample without fixation. The ligation product generated from the ligation of (i) a first probe bound to a first analyte-binding moiety to (ii) a second oligonucleotide bound to a second analyte-binding moiety based on the proximity of the first and second analyte binding moieties is used to determine the location of an interaction between at least one nucleic acid and at least one non-nucleic acid analyte in a biological sample. In some forms, the described methods include simultaneously characterizing multiple different analyte interactions (e.g., a multiplicity of interactions between one target protein that binds to multiple different nucleic acids) in a single biological sample.

In some forms, the disclosed methods utilize a first probe including a binding moiety that targets a nucleic acid-binding protein, and a second probe that includes a region of complementarity with a nucleic acid in the sample as well as a region that specifically binds to or forms part of a splinting region with the first probe. The second probe will bind to the first probe to form a chimeric, combined probe only when the first and second probes interact with their respective targets in proximity to one another (e.g., within about 300 μm, 200 μm, 100 μm, 50 μm, 10 μm, or 1 μm, or less than 1 μm, such as 900 nm, 800 nm, 700 nm, 500 nm, 400 nm of each other (e.g., about 300 nm, about 200 nm, about 150 nm, about 100 nm, about 50 nm, about 25 nm, about 10 nm, or about 5 nm)).

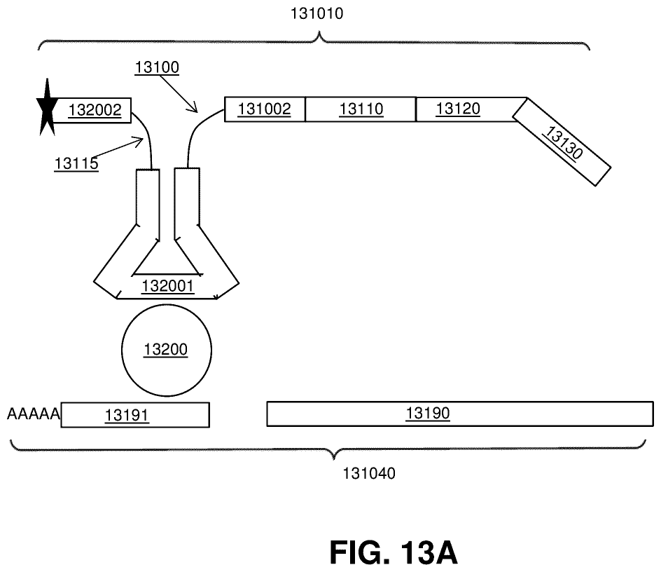

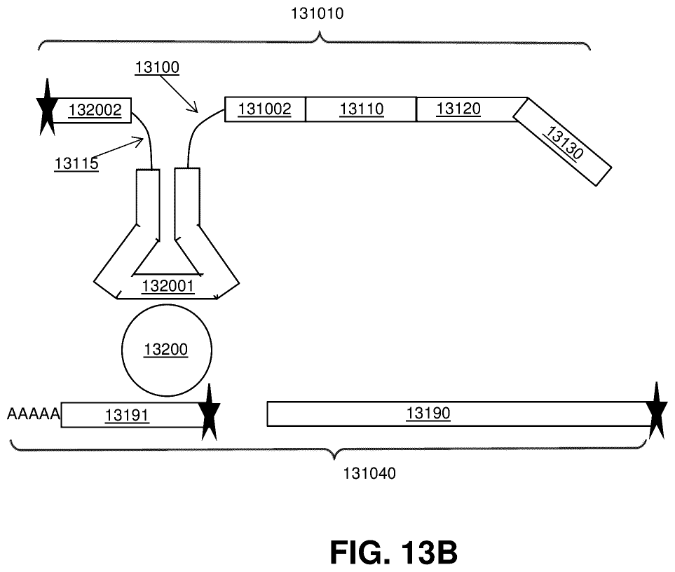

In some forms, the first probe includes a target protein-binding moiety that is or includes a nucleic acid, such as an aptamer, or a protein, such as an antibody, or antigen binding fragment thereof. Typically, the first probe includes a target protein-binding moiety that binds to a protein in a sample, such as a nucleic acid binding protein. In some forms, the first probe includes a target protein-binding moiety that specifically binds a protein at the cell surface (e.g., any of the exemplary cell surface analytes described herein). In some forms, the first probe includes a target protein-binding moiety that metabolically binds a specific target protein. In some forms, the first probe includes a target protein-binding moiety that enzymatically binds a specific target protein. See, for example, FIGS. 13A-13D. In some forms, when the first probe includes an antibody as a target protein-binding moiety (i.e., a “primary antibody”), the methods may further include one or more steps to bind a secondary antibody to the primary antibody. In some forms, the methods include steps to wash away and/or otherwise remove any un-bound primary antibodies from the sample before a secondary antibody is added. In some forms, the unbound secondary antibodies are washed away before any one or more additional downstream steps. Wash steps can be performed using any suitable wash solution, exemplary forms of which are disclosed herein (e.g., 1×PBST).

The second probe includes one or more sequence(s) that hybridizes or otherwise selectively binds to a nucleic acid analyte in the sample. The sequence can be a random sequence or a designed sequence. In some forms, the methods include a pool of two or more species of second probes, for example, including different sequence(s) that hybridize or otherwise selectively bind a target nucleic acid. Therefore, in some forms, a second probe of a plurality of probes includes a sequence that is a random sequence or a designed sequence.

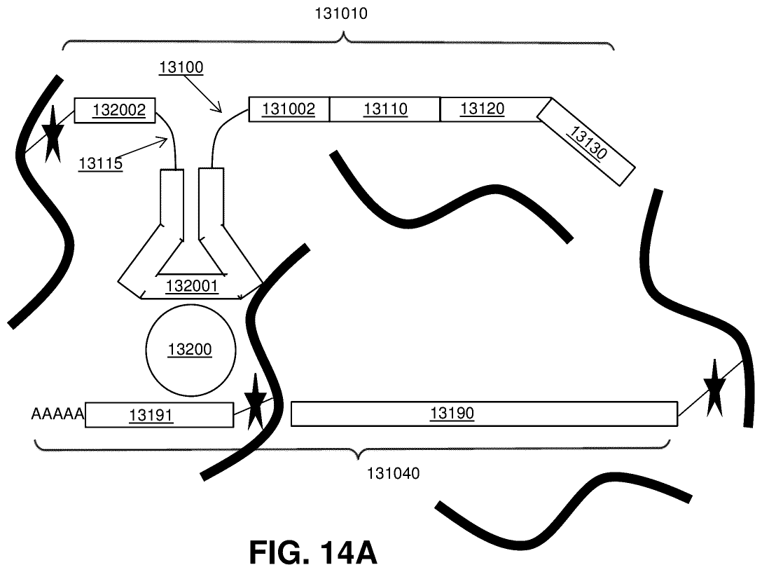

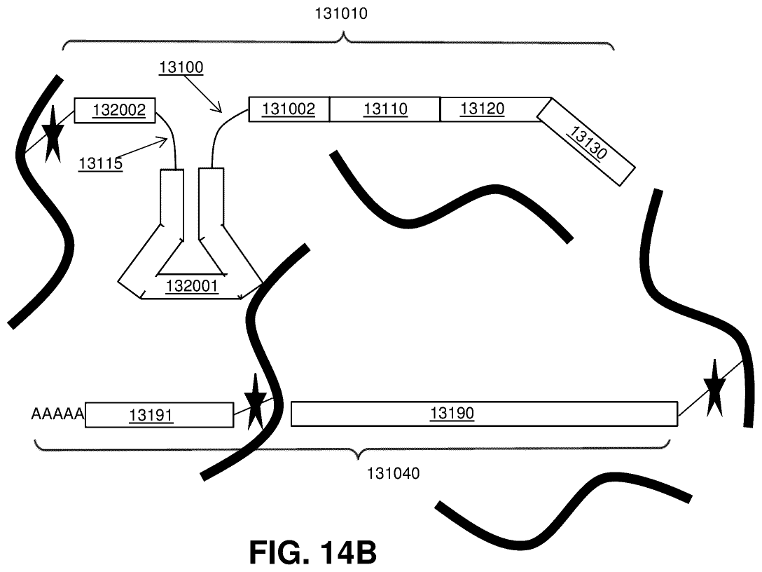

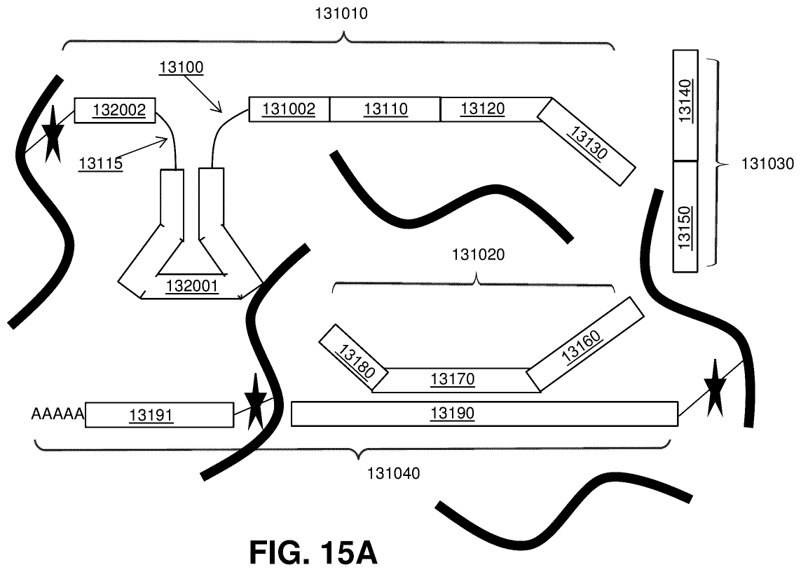

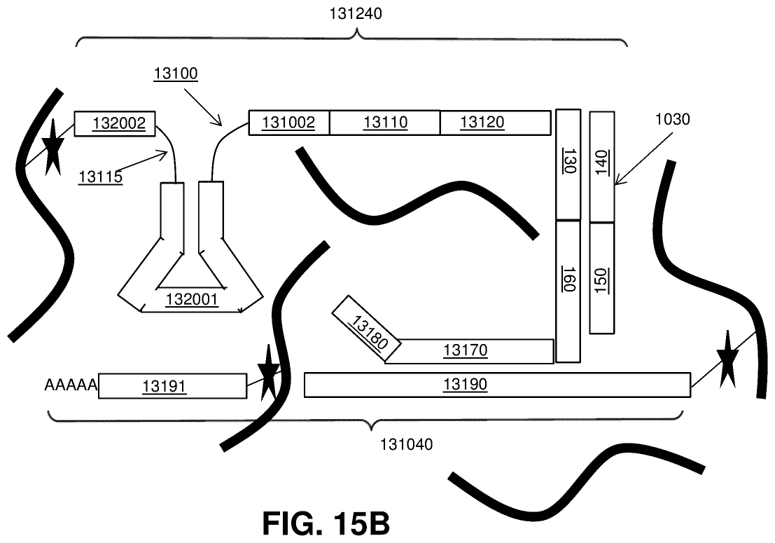

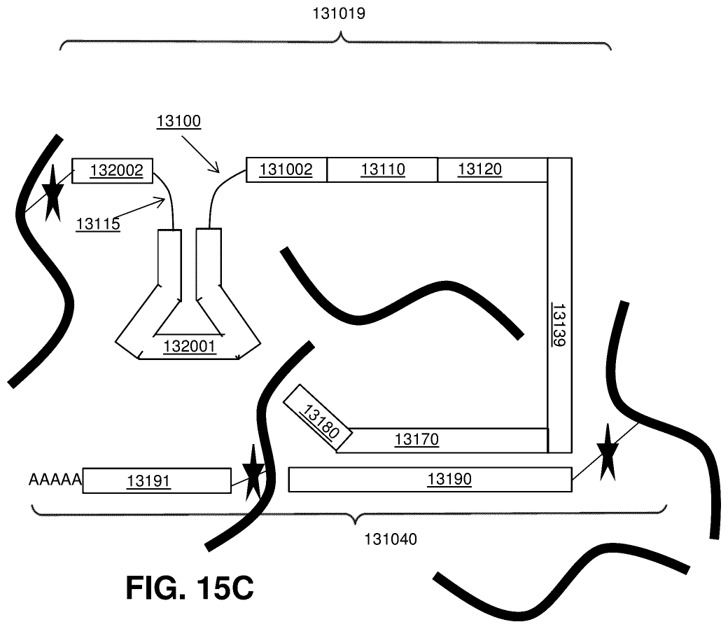

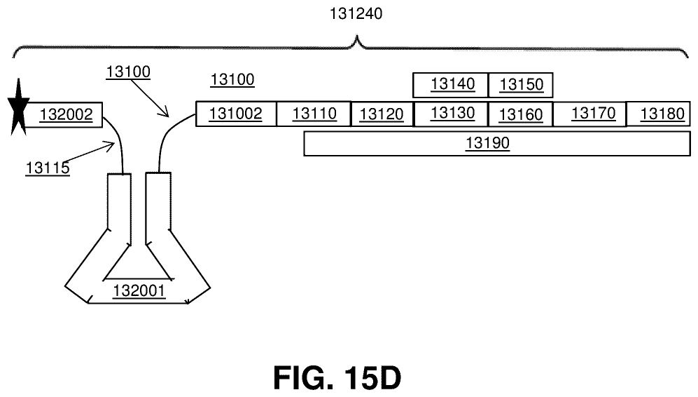

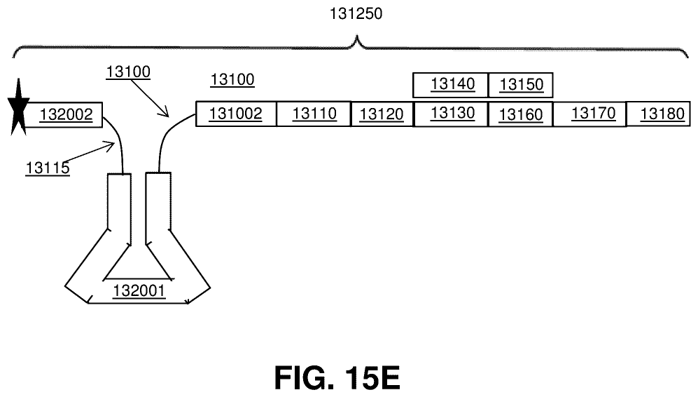

The methods include one or more steps to hybridize and/or ligate the immobilized “fixed” first probe embedded within a gel and second probe to yield a fixed chimeric probe including sequence information corresponding to both the target protein and the target nucleic acid, which is captured by capture probes, such as arrayed capture probes including spatial information. The target protein and nucleic acid must interact together or be in proximity to each other to dock together and form a chimeric combined probe; for example, in some forms, the distance between the docking sites on the first probe and the second probe, when bound to the target protein and target nucleic acid, respectively, is the determining factor in formation of a combined chimeric probe. In some forms, the methods employ a splint oligonucleotide and optionally a ligation step to assist formation of a combined probe. See, for example, FIGS. 14A-14B, 15A-15B.

### 1. General Methodology

Generally, the methods include all or some of steps (a)-(h), as follows.

A first step (step a) generally includes contacting a biological sample including a plurality of nucleic acids with a plurality of first probes. Each first probe typically includes at least the components (i) and (ii), as follows:
(i) a target protein-binding moiety and an associated first oligonucleotide including a second-probe docking sequence and, optionally, a target protein identification barcode, and
(ii) one or more functional group(s).

The one or more functional group(s) can be used for gel embedding. Typically, the contacting is carried out under conditions suitable for the target protein-binding moiety to bind to a target protein within a biological sample.

A second step (step b) generally includes modifying a 3′ and/or 5′ end of the nucleic acids in the sample with one or more functional group(s), e.g., for embedding within a permeable matrix, such as a permeable gel or hydrogel.

A third step (step c) generally includes embedding the biological sample in a permeable matrix, such as a permeable gel or hydrogel. The process of embedding generally includes forming an interaction between the one or more functional group(s) and the matrix.

A fourth step (step d) generally includes hybridizing a plurality of second probes to the plurality of nucleic acids to form a plurality of hybridized second probes. Each second probe typically includes at least the components (i) and (ii) and (iii), as follows: (i) a first-probe docking sequence; (ii) a region complementary to one or more nucleic acids of the plurality of nucleic acids; and (iii) a capture domain binding sequence.

A fifth step (step e) generally includes combining the first probe and the hybridized second probe to form a combined probe including a capture domain binding sequence. In some forms, the methods include hybridizing the first splint oligonucleotide to both the first probe docking sequence and the second-probe docking sequence, and optionally ligating the first probe and second hybridized probes together to form the combined probe, for example, by contacting the sample with a ligase enzyme. In some forms, the first splint oligonucleotide includes one or more additional nucleic acid residues between the sequence complementary to the first probe docking sequence and the sequence complementary to the second-probe docking sequence. Typically, the size and/or sequence of the first-probe docking sequence, and/or second-probe docking sequence, and/or the first splint oligonucleotide are configured so that combining the first probe and hybridized second probe will only occur when the target protein is bound directly to the nucleic acid.

A sixth step (step f) generally includes releasing the combined probe, e.g., from the sample and/or matrix. Releasing typically involves contacting the sample with one or more agents that digests, dissolves, or otherwise removes the non-nucleic acid components in the sample. Exemplary non-nucleic acid components include proteins, carbohydrates, lipids, and combinations thereof. In some forms, the step of releasing removes dyes, labels, small molecules, etc., from the embedded sample.

A seventh step (step g) generally includes hybridizing the capture domain binding sequence of the combined probe to the capture domain of a capture probe, such as an arrayed capture probe. Each capture probe typically includes at least the components (i) and (ii), as follows: (i) a spatial barcode; and (ii) a capture domain. To prevent premature or non-specific binding to the capture probe, the methods can employ blocking probes to reduce, block or otherwise prevent interaction between the capture probe binding domain of a combined probe and a capture domain of a capture probe. In some forms, the methods prevent premature binding to the capture probe by altering the temperature and/or other conditions to limit or prevent hybridization of the capture domain binding sequence to the capture domain of a capture probe until desired. Suitable conditions and buffers for washing unbound protein binding moieties away from the sample, as well as hybridization conditions are described herein.

An eighth step (step h) generally includes determining one or more of (hi) the spatial barcode sequence or a complement thereof; (hii) all or a portion of the hybridized second probe sequence (e.g., corresponding to a nucleic acid in the plurality of nucleic acids) or a complement thereof; and/or (hiii) the sequence of the target protein identification barcode.

A ninth step (step i) generally includes using the determined sequences of (hi), and/or (hii) and/or (hiii) to identify the location and/or abundance of the interaction between the target protein and nucleic acid in the biological sample.

In some forms, the methods include one or more steps to remove the target protein binding moiety from the first probe. For example, following hybridization of the combined probe to the capture probe in step (g). For example, if the target protein binding moiety is attached to the first oligonucleotide via a cleavable linker, in some forms the methods include one or more steps to cleave the cleavable linker. For example, if the cleavage domain includes a disulfide bond, in some forms the methods include treatment with a reducing agent and detergent washing, chaotropic salt treatment, etc., to cleave the linker.

It is contemplated that, prior to the commencement of the described steps (a)-(i), the methods include one or more “pre-processing” steps, for example, to bake, dewax, rehydrate, H and E stain, BF image and Destain a sample. An exemplary workflow for pre-processing of a sample is depicted in the first six steps of the exemplary workflow of FIG. 17. Typically, when the method includes one or more steps for pre-processing a sample, the pre-processing provides a biological sample, such as a tissue sample, in an amount and format suitable for spatial analysis of nucleic acid binding protein/nucleic acid interactions, according to the described method steps (a)-(i).

(a) Contacting a Biological Sample Including a Plurality of Nucleic Acids with a Plurality of First Probes

The methods include contacting a biological sample including a plurality of nucleic acids with a plurality of first probes. Each first probe typically includes at least the components (i) and (ii), as follows: (i) a target protein-binding moiety and an associated first oligonucleotide including a second-probe docking sequence and, optionally, a target protein identification barcode, and (ii) one or more functional group(s), e.g., which can be used for matrix (e.g., gel) embedding. Typically, the contacting is carried out under conditions suitable for the target protein-binding moiety to bind to a target protein.

In some forms, the methods include one or more wash steps to remove unbound first probes prior to an embedding step. Typically, when the methods include one or more wash steps to remove unbound first probes, the wash steps occur prior to step (b), or prior to step (c), or prior to adding the second probes.

In some forms, the methods determine a location and/or abundance of an interaction between a known target protein with a known nucleic acid in a biological sample obtained from a subject. Multiple specific complexes of nucleic acids, such as RNA, interacting with nucleic acid binding proteins, such as RNA binding proteins, are known in the art, as described below. Interpreting the location and/or abundance of these specific interactions within a tissue sample provides a wealth of data that is valuable for analysis, diagnosis, and monitoring of several diseases. Therefore, in some forms, when the interaction between a target protein and its nucleic acid binding partner are specifically sought within a tissue, the methods implement reduced numbers of steps and/or oligonucleotides having reduced complexity. For example, in some forms, when the methods target interactions with a specific protein, only a single protein binding moiety need be used, and when the target nucleic acid is a specific nucleic acid, incorporation of all or part of the sequence of the nucleic acid in the combined probe and determination of all or part of its sequence is not necessary and is thus optional. Therefore, in some forms, when the methods determine a location and/or abundance of an interaction between a known target protein with a known nucleic acid, the methods implement a streamlined process with a reduced number of steps and/or species of probes having reduced complexity, as compared to those implemented in methods for which one or both the target protein and target nucleic acid are unknown or non-specifically bound to the oligonucleotides.

In some forms, the step of contacting further includes one or more steps of fragmenting the nucleic acids in the biological sample. The fragmenting can occur immediately prior to the contacting, during or after the contacting. For example, in some forms, the nucleic acids in a sample are fragmented prior to step (a). In other forms, the fragmenting occurs after or during any of the other method steps, for example, after or during any of steps (b), (c), (d) or (e). In some forms, the fragmenting of nucleic acids occurs as a result of one or more other processing steps. In other forms, the fragmenting includes contacting the sample with one or more nuclease enzymes. Exemplary nuclease enzymes include endonucleases.

(b) Modifying Nucleic Acids in the Sample with One or More Functional Group(s) for Embedding

The methods include modifying a 3′ and/or 5′ end of the nucleic acids in the sample with one or more functional group(s) for embedding within a permeable matrix, such as a permeable gel or hydrogel. In some aspects, the analytes, polynucleotides and/or amplification product (e.g., amplicon) of an analyte or a probe bound thereto can be anchored to a polymer matrix. For example, the polymer matrix can be a gel (e.g., hydrogel). In some forms, one or more of the target analytes (e.g., nucleic acids), analyte proxies and/or amplification products (e.g., amplicons) thereof can be modified to contain functional groups that can be used as an anchoring site to attach the target analytes, analyte proxies and/or amplification products to a polymer matrix.

An exemplary functional group(s) for embedding within a matrix is acrydite. Another exemplary functional group includes thiolated primers and methylsulphone-acrylate monomers which can be spiked into the matrix. In some forms, the one or more functional groups is a 5′-phosphate group or a 5′-phosphate group modified with a leaving group.

In some forms, the matrix is a dissolvable gel matrix or hydrogel. An exemplary dissolvable hydrogel includes N,N′-(1,2-Dihydroxyethylene)bis-acrylamide that is dissolvable upon incubation with a periodate.

In some forms, the target nucleic acids are tethered to the matrix via a boronate ester bond. In some forms, the boronate ester bond is formed between a boronic acid moiety and 3′ diols of the target nucleic acid which is an RNA. In some forms, the matrix includes a boronic acid-based hydrogel matrix.

In some forms, the methods include contacting a biological sample including a target nucleic acid with an attachment agent including a boronic acid moiety capable of covalently reacting with at least one 2′,3′ vicinal diol of the target nucleic acid and an attachment moiety capable of attaching covalently or noncovalently to a matrix-forming agent in the biological sample, wherein the biological sample and the attachment agent are contacted under conditions suitable to form a covalent bond between the boronic acid moiety and the 2′,3′ vicinal diol of the target nucleic acid.

In some forms, the methods include contacting the biological sample including a target nucleic acid with a 3′ phosphatase to provide a target nucleic acid including a 2′,3′-vicinal diol moiety; contacting the biological sample with a formylation reagent, wherein the formylation reagent converts the 2′,3′-vicinal diol moiety into a 2′3′-dialdehyde moiety, optionally wherein the formylation reagent includes sodium (meta) periodate; contacting the biological sample with an attachment agent and a matrix forming agent, the attachment agent including at least one aldehyde-reactive group capable of forming a covalent bond with at least one aldehyde of the 2′,3′-dialdehyde moiety of the fragmented target nucleic acid and an attachment moiety capable of attaching covalently or noncovalently to the matrix-forming agent, thereby forming a matrix embedding the biological sample and tethering the fragmented target nucleic acid to the matrix. In some forms, RNA tethering to the matrix can use boronic acid moieties in the matrix to passively capture 3′ RNA ends that have been polished, where the tethering occurs when the pH is greater than the pKa of the boronic acid moiety (which induces the formation of boronate esters with 3′ RNA diols).

In some forms, the methods include contacting a biological sample having a ribonucleic acid with a formylation reagent, wherein the ribonucleic acid includes a 2′,3′-vicinal diol and the formylation reagent reacts with the 2′,3′-vicinal diol moiety to provide a ribonucleic acid including at least one aldehyde moiety, optionally whereby the ribonucleic acid includes a dialdehyde moiety. The term “formylation” generally refers to the addition of a formyl or aldehyde group to a chemical or biological entity. As utilized herein, the term “formylation reagent” refers to any suitable chemical and/or biological reagent capable of performing formylation on a ribonucleic acid and, more specifically in the context of the present disclosure, the formylation reagents as provided herein are capable of converting the 2′,3′-vicinal diol on the terminal 3′ end ribose ring of a ribonucleic acid into a ribonucleic acid including at least one aldehyde. In some forms, the formylation reagent reacts with the 2′,3′-vicinal diol to form at least one aldehyde moiety. In some forms, the formylation reagent is an oxidant, the formylation reagent reacts with the 2′,3′-vicinal diol to form two aldehyde moieties.

### (c) Embedding the Biological Sample in a Permeable Matrix, Such as a Permeable Gel or Hydrogel

The methods include embedding the biological sample (also referred to as “sample” herein) in a permeable matrix, such as a permeable gel or hydrogel. The process of embedding generally includes forming an interaction between the one or more functional group(s) and the matrix. Embedding the biological sample in this manner typically involves contacting the biological sample with a gel (e.g., hydrogel) or gel forming agent(s) such that the biological sample becomes surrounded by the gel (e.g., hydrogel). For example, the sample can be embedded by contacting the sample with a suitable polymer material, and activating the polymer material to form a hydrogel. In some forms, the hydrogel is formed such that the hydrogel is internalized within the biological sample. Biological samples can include analytes (e.g., protein, RNA, and/or DNA) embedded in a 3D matrix. In some forms, the hydrogel is formed such that the hydrogel is internalized within the biological sample. In some forms, the biological sample is immobilized in the hydrogel via cross-linking of the polymer material that forms the hydrogel. Cross-linking can be performed chemically and/or photochemically, or alternatively by any other suitable hydrogel-formation method.

A hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some forms, the biological sample is reversibly cross-linked. A hydrogel may include a macromolecular polymer gel including a network. Within the network, some polymer chains can optionally be cross-linked, although cross-linking does not always occur.

In some forms, the gel is a hydrogel including a boronic acid moiety. For example, in some forms, a hydrogel including a boronic acid moiety can be used to tether a nucleic acid (e.g., RNA, DNA, first probe, second probe) of a sample (e.g., a cell or tissue sample) embedded in the hydrogel for subsequent removal of proteins (e.g., ribosome components) and lipids. A drop in pH below the pKa of the boronic acid moieties in the tethered sample can release the RNAs or other nucleic acid analyte to begin interfacing with the arrayed capture probes. In some forms, a slightly acidic buffer is used to un-tether the nucleic acid (e.g., RNA), where the proximity of the array capture probes helps limit lateral diffusion through the hydrogels to mitigate mis-localization. In some forms, capacitance can be used to drive migration of the newly liberated nucleic acid (e.g., RNA) towards the array of capture probes.

In some forms, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference. The composition and application of the hydrogel-matrix to a biological sample can vary. As one example, where the biological sample is a tissue section, the hydrogel-matrix can include a monomer solution and an ammonium persulfate (APS initiator/tetramethylethylenediamine (TEMED)) accelerator solution. As another example, where the biological sample contains cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm. Additional methods and aspects of hydrogel embedding of biological samples are described for example in Chen et al., Science 347(6221):543-548, 2015, the entire contents of which are incorporated herein by reference.

In some forms, hydrogel formation occurs within a biological sample. In some forms, a biological sample (e.g., tissue section) is embedded in a hydrogel. In some forms, hydrogel subunits are infused into the biological sample, and polymerization of the hydrogel is initiated by an external or internal stimulus.

In some forms, the step of functionalizing the nucleic acids and the step of embedding the sample within a permeable matrix occur simultaneously, according to the method that is applied for the embedding process.

In some forms, a 3D matrix may include a network of natural molecules and/or synthetic molecules that are chemically and/or enzymatically linked, e.g., by crosslinking. In some forms, a 3D matrix may include a synthetic polymer. In some forms, a 3D matrix includes a hydrogel.

In some forms, the methods include embedding the biological sample in a matrix, tethering a target nucleic acid in the biological sample to the matrix; and clearing at least a subset of proteins, lipids, or other cellular components in the biological sample from the matrix. In some forms, clearing can include contacting the biological sample with one or more detergents and/or enzymes (e.g., lipases, proteases, cellulases).

The modified nucleic acid molecules can be present in the biological sample (such as cellular DNA or RNA) or applied to the biological sample (such as a nucleic acid probe targeting a cellular DNA or RNA) or generated in the biological sample as a proxy of a cellular DNA or RNA (e.g., ligated RTL probes). In some forms, a method disclosed herein further includes removing at least a subset of the proteins, lipids, or other cellular components in the biological sample from the matrix, thereby leaving the nucleic acid molecules tethered to the matrix. In some forms, the tethered nucleic acid molecules can be untethered and transferred to a spatial array on a substrate. In some forms, the spatial array includes a plurality of features and wherein each feature is associated with a unique spatial location on the array. Subsequent analysis of transferred nucleic acid molecules to the spatial array can include determining the identities of the transferred nucleic acid molecules (and the identities of the corresponding analytes) and the spatial location of the transferred nucleic acid molecules (and the spatial locations of the corresponding analytes) within the original biological sample. Since the array and the biological sample can be aligned (e.g., such that a location in the biological sample corresponds to the location of a feature within the array), the spatial location of an analyte or analyte interactions within the biological sample can be determined based on the feature to which the corresponding nucleic acid molecule is bound (e.g., directly or indirectly) on the array, and the feature's relative spatial location within the array, which is determined by the spatial barcodes on the capture probes at each feature to which the transferred nucleic acid molecules are hybridized or ligated. Thus, each feature can include capture probes having the same spatial barcode where the spatial barcode differs from feature to feature. In some forms, the features include a plurality of beads.

In some forms, the matrix is a hydrogel matrix. In some forms, the tethering includes tethering a 3′ end of the target nucleic acid to the matrix. In some forms, the tethering includes tethering a 5′ end of the target nucleic acid to the matrix. In some forms, the tethering includes enzymatic tethering. In some forms, the tethering includes non-enzymatic tethering. In some forms, the tethering includes a periodate oxidation reaction or a reaction with boronic acid.

In some forms, the tethering includes tethering the target nucleic acid or other nucleic acid (e.g., first probe, second probe) to the matrix via a linker. In some forms, the linker includes a cleavable linker. In some forms, the cleavable linker includes a disulfide bond. In some forms, the matrix is formed using N,N′-Bis(acryloyl) cystamine (BAC) as a crosslinker. In some forms, the linker is a photocleavable linker.

In some forms, when the attachment agent includes a boronic acid moiety capable of covalently reacting with at least one 2′,3′ vicinal diol of the target nucleic acid and an attachment moiety capable of attaching covalently or noncovalently to a matrix-forming agent in the biological sample, the biological sample and the attachment agent are contacted under conditions suitable to form a covalent bond between the boronic acid moiety and the 2′,3′ vicinal diol of the target nucleic acid. For example in some forms, the methods include contacting the biological sample with a matrix forming agent, thereby forming a matrix embedding the biological sample and tethering the target nucleic acid to the matrix.

In some forms, after contacting a biological sample with a first probe that includes a target analyte binding moiety, a removal step can optionally be performed to remove all or a portion of the biological sample. Typically, the removal step(s) include clearing the embedded biological sample from the matrix. In some forms, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some forms, the removal step can include ablation of the tissue (e.g., laser ablation).

In some forms, the releasing includes contacting the matrix with a chemical agent configured to cleave a cleavable linker between the target nucleic acid and the matrix. In some forms, the releasing includes illuminating the matrix with light, thereby cleaving a photocleavable linker between the target nucleic acid and the matrix. In some forms, the releasing includes altering the pH of a solution in contact with the matrix, thereby releasing the tethered target nucleic acid. In some forms, when the matrix includes a boronic acid-based hydrogel matrix, the releasing includes exposing the matrix to heat, wherein the matrix is a hydrogel matrix including poly(N-isopropylacrylamide), thereby releasing the tethered target.

Generally, biological samples embedded in hydrogels can be cleared using any suitable method. For example, electrophoretic tissue clearing methods can be used to remove biological macromolecules from the hydrogel-embedded sample. In some forms, a hydrogel-embedded sample is stored before or after clearing of hydrogel, in a medium (e.g., a mounting medium, methylcellulose, or other semi-solid mediums). In some forms, when the embedding is reversible, the nucleic acids can remain tethered during sample clearing, and the tethering can be reversed and/or the nucleic acids (or a proxy thereof) be released for subsequent analysis. In some forms, dissolvable gel formulations can be used, such that nucleic acids can remain tethered to the hydrogel during sample clearing, whereas the hydrogel can be dissolved to release the nucleic acids after sample clearing. For example, in some forms, RNA tethering to the matrix can use boronic acid moieties in the matrix to passively capture 3′ RNA ends that have been polished, where the tethering occurs when the pH is greater than the pKa of the boronic acid moiety (which induces the formation of boronate esters with 3′ RNA diols) and the tethering is reversed when the pH is below than the pKa.

In some forms, a hydrogel including a boronic acid moiety can be used to tether an RNA of a sample (e.g., a cell or tissue sample embedded in the hydrogel) for subsequent removal of proteins (e.g., ribosome components) and lipids. A drop in pH below the pKa of the boronic acid moieties in the tethered sample can release the RNAs or other nucleic acid analyte to begin interfacing with the arrayed capture probes. In some forms, a slightly acidic buffer is used to un-tether the RNA, where the proximity of the array capture probes helps limit lateral diffusion through the hydrogels to mitigate mis-localization. In some forms, capacitance can be used to drive migration of the newly liberated RNA towards the array.

### (1) Tissue Removal/Clearing

In some forms, after contacting a biological sample with a first probe that includes a target analyte binding moiety, a tissue removal step can optionally be performed to remove all or a portion of the biological sample. Typically, the removal step(s) include clearing the embedded biological sample from the matrix. In some forms, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some forms, the removal step can include ablation of the tissue (e.g., laser ablation).

In some forms, the releasing includes contacting the matrix with a chemical agent configured to cleave a cleavable linker between the target nucleic acid and the matrix. In some forms, the releasing includes illuminating the matrix with light, thereby cleaving a photocleavable linker between the target nucleic acid and the matrix. In some forms, the releasing includes altering the pH of a solution in contact with the matrix, thereby releasing the tethered target nucleic acid. In some forms, when the matrix includes a boronic acid-based hydrogel matrix, the releasing includes exposing the matrix to heat, wherein the matrix is a hydrogel matrix including poly(N-isopropylacrylamide), thereby releasing the tethered target.

Generally, biological samples embedded in hydrogels can be cleared using any suitable method. For example, electrophoretic tissue clearing methods can be used to remove biological macromolecules from the hydrogel-embedded sample. In some forms, a hydrogel-embedded sample is stored before or after clearing of hydrogel, in a medium (e.g., a mounting medium, methylcellulose, or other semi-solid mediums). In some forms, when the embedding is reversible, the nucleic acids can remain tethered during sample clearing, and the tethering can be reversed and/or the nucleic acids (or a proxy thereof) be released for subsequent analysis. In some forms, dissolvable gel formulations can be used, such that nucleic acids can remain tethered to the hydrogel during sample clearing, whereas the hydrogel can be dissolved to release the nucleic acids after sample clearing. For example, in some forms, RNA tethering to the matrix can use boronic acid moieties in the matrix to passively capture 3′ RNA ends that have been polished, where the tethering occurs when the pH is greater than the pKa of the boronic acid moiety (which induces the formation of boronate esters with 3′ RNA diols) and the tethering is reversed when the pH is below than the pKa.

In some forms, a hydrogel including a boronic acid moiety can be used to tether an RNA of a sample (e.g., a cell or tissue sample embedded in the hydrogel) for subsequent removal of proteins (e.g., ribosome components) and lipids. A drop in pH below the pKa of the boronic acid moieties in the tethered sample can release the RNAs or other nucleic acid analyte to begin interfacing with the arrayed capture probes. In some forms, a slightly acidic buffer is used to un-tether the RNA, where the proximity of the array capture probes helps limit lateral diffusion through the hydrogels to mitigate mis-localization. In some forms, capacitance can be used to drive migration of the newly liberated RNA towards the array. When the methods include a step of tissue releasing/permeabilizing the sample following embedding within a matrix/gel, the “sample” becomes nucleic acids present/distributed within a hydrogel or matrix, whereby the spatial arrangement or each nucleic acid is maintained with respect to the original position in the original sample.

### (d) Hybridizing Second Probes to the Nucleic Acids to Form a Plurality of Hybridized Second Probes

The methods include contacting a biological sample including a plurality of nucleic acids with a plurality of second probes. Typically, the methods include hybridizing a plurality of second probes to the plurality of nucleic acids to form a plurality of hybridized second probes. Each second probe typically includes at least the components (i) and (ii) and (iii), as follows: (i) a first probe docking sequence; (ii) a region complementary to one or more nucleic acids of the plurality of nucleic acids; and (iii) a capture domain binding sequence.

In some forms, the methods include, subsequent to, or during step (d), one or more wash steps to remove unbound second probes from the sample.

### (e) Combining First Probes and Hybridized Second Probes to Form a Chimeric, Combined Probe

The methods include combining the first probe and the hybridized second probe to form a combined probe including a capture domain binding sequence. Typically, the combining occurs through hybridization of the first and second probes together in the region of the first and second probe docking domains. In some forms, combining the first and second probes includes the use of a first splint oligonucleotide that includes a sequence complementary to the first probe docking sequence; and a sequence complementary to the second probe docking sequence.

### (f) Releasing the Combined Probe

The methods include releasing the combined probe, e.g., from the target nucleic acid and/or the matrix (e.g., via the functional group(s)). In some forms, releasing the combined probe includes releasing the nucleic acids in the biological sample by removal of all the non-nucleic acid components. In other forms, when a first tissue removal step has already been carried out, the methods release the combined probe from the RNA target analyte.

In some forms, releasing involves permeabilizing the sample. When the combined probe is within a tissue sample that has not previously been permeabilized, the step of releasing a combined probe includes contacting the sample with one or more agents that digests, dissolves, or otherwise removes the non-nucleic acid components in the sample. Exemplary non-nucleic acid components include proteins, carbohydrates, lipids, and combinations thereof. In some forms, the step of releasing removes dyes, labels, small molecules, etc., from embedded the sample. In some forms, the step of releasing includes degrading or otherwise removing the one or more nucleic acids hybridized to the combined probe.

For example, in some forms, permeabilizing includes contacting the biological sample with a permeabilization reagent. In some forms, a permeable matrix, such as a gel, includes a permeabilization reagent. Exemplary permeabilizing reagents include organic solvents, detergents, and enzymes, or a combination thereof. In some forms, the step of releasing includes contacting the sample with a reagent selected from the group consisting of an endopeptidase, a protease, sodium dodecyl sulfate (SDS), polyethylene glycol tert-octylphenyl ether, polysorbate 80, polysorbate 20, N-lauroylsarcosine sodium salt solution, saponin, Triton X-100™, and Tween-20™. In some forms, the step or releasing includes contacting the sample with a protease. In other forms, the combined probe is present within a permeabilized sample, i.e., a sample that has been treated to remove all of the non-nucleic acid components, prior to formation of the combined probe. In some forms, the step or releasing further includes one or more steps to degrade or otherwise remove one or more nucleic acids hybridized to the combined probe. Therefore, in some forms, the step of releasing includes contacting the sample with one or more nuclease enzymes, such as a DNAase or an RNAse. An exemplary RNAse is RNAse H.

In preferred forms, the step of releasing includes releasing the combined probe from the target nucleic acid via contacting the sample with an RNAse (e.g., RNAse H).

### (g) Hybridizing the Combined Probe to the Capture Domain of a Capture Probe

The methods include hybridizing the capture domain binding sequence of the combined probe to the capture domain of a capture probe, such as an arrayed capture probe. Each second probe typically includes at least the components (i) and (ii), as follows: (i) a spatial barcode; and (ii) a capture domain.

In some forms, the methods can include a step of contacting a biological sample or bringing the biological sample into proximity with a substrate including a plurality of capture probes, whereby a capture probe of the plurality of capture probes includes a spatial barcode and a capture domain. Typically, the methods include contacting or aligning the sample or the matrix (e.g., gel) with a substrate having bound thereto arrayed capture probes prior to step (g). In other forms, the methods include contacting or aligning the sample with a substrate having bound thereto arrayed capture probes subsequent to, or during step (f). In some forms, to prevent premature or non-specific binding to the capture probe, the methods can employ blocking probes to block or otherwise prevent interaction between the capture probe binding domain of a combined probe and a capture domain of a capture probe. In other forms, e.g., when the tissue is on a first substrate (e.g., a first glass slide) and the array of capture probes is on a second substrate (e.g., a second glass slide), and the two are brought into proximity for capture of the analyte onto the array, the methodology precludes the requirement for blocking probes. In some forms, the methods prevent premature binding to the capture probe by altering the temperature and/or other conditions to limit or prevent hybridization of the capture domain binding sequence to the capture domain of a capture probe until desired. Suitable conditions and buffers for washing unbound protein binding moieties away from the sample, as well as hybridization conditions are described herein.

### (h) Sequencing of Captured Probes

The methods include determining one or more of (hi) the spatial barcode sequence or a complement thereof; (hii) all or a portion of the hybridized second probe sequence (e.g., corresponding to a sequence of a target nucleic acid) or a complement thereof; and/or (hiii) the sequence of the target protein identification barcode.

In some forms, one or both of the first and second and capture probes includes a functional domain that facilitates extension, and/or amplification and/or sequence analysis of the capture probe hybridized to the combined probe. Therefore, in some forms, sequencing information is collected only from capture probes hybridized to a combined probe, or the downstream nucleic acids (e.g., amplicons) generated therefrom.

In some forms, the methods include one or more steps to remove the target protein binding motif from the first probe. For example, following hybridization of the combined probe to the capture probe in step (g). For example, if the target protein binding motif is attached to the first oligonucleotide via a cleavable linker, in some forms the methods include one or more steps to cleave the cleavable linker. For example, if the cleavage domain includes a disulfide bond, in some forms the methods include treatment with a reducing agent and detergent washing, chaotropic salt treatment, etc., to cleave the linker.

### (i) Determination of Spatial Information

The methods include using the determined sequences of (hi), and/or (hii) and/or (hiii) to identify the location and/or abundance of the interaction between the target protein and nucleic acid in the biological sample.

### 2. Methods Employing a Splint Oligonucleotide

In some forms, the methods employ a splint oligonucleotide to act as a splint assisting formation of the combined chimeric probe formed by docking of the first and second probes. When the methods include a splint oligonucleotide, the first and second probes each include a region of complementarity (i.e., a “docking region”) of the splint oligonucleotide, such that the first and second probes both hybridize to the same splint oligonucleotide. See, for example, FIGS. 15A-15B. The respective docking regions of the splint oligonucleotide can be contiguous or non-contiguous, for example, separated by one or more bases from one another. In some forms, the methods include a ligation step, to ligate the ends of the first and second prior probes together (i.e., when the respective docking regions of the splint oligonucleotide are contiguous) or to one or more “filling” nucleotides (i.e., when the respective docking regions of the splint oligonucleotide are non-contiguous). Typically, the ligation step includes contacting the matrix with one or more enzymes and reagents under conditions suitable for ligation.

As described, the regions of complementarity on the splint oligonucleotide can be directly adjacent or can be separated by one or more nucleotides. When the regions of complementarity on the third oligonucleotide do not abut directly, but are separated by a gap of one or more nucleotides, the methods also include one or more steps to fill in the gap following hybridization of the first and second oligonucleotides to the third oligonucleotide. Therefore, in some forms, the methods include use of a polymerase enzyme to fill in the gap, using the third oligonucleotide as a template.

An exemplary method including a splinting oligonucleotide, includes:
Steps (a)-(d) as described above;
(e) contacting the sample with a plurality of splint oligonucleotides and combining the first probe and the hybridized second probe with the splint oligonucleotide to form a combined probe including a capture domain binding sequence; and optionally contacting the sample with a ligase enzyme under conditions suitable for a ligation reaction between an end of the first probe and an end of the second probe. In some forms, upon hybridization with the splint oligonucleotide, the ends of the first and second probes do not abut directly but are separated by a gap of several nucleotides. Therefore, in some forms, the methods include one or more steps to fill in the gap following hybridization of the first and second probes to the splint oligonucleotide. Typically, the methods to fill in the gap include use of a polymerase enzyme, using the splint oligonucleotide as a template. For example, in an exemplary form, ligating the first probe and the second probe to provide a ligation product with the assistance of a splint oligonucleotide, includes one or more steps of filling in a gap of between one and one hundred nucleotides, such as between 4 and 30 nucleotides, between the ends of the first and second probes hybridized to the splint oligonucleotide to create an intact chimeric combined probe. In some forms, when the methods include a splint oligonucleotide, the splint includes all or part of an additional functional domain, or the complement thereof, such that the ligated combined probe will include the additional functional domain. In some forms, the methods require one or more steps of filling in the gap of between one and one hundred nucleotides, such as between 4 and 30 nucleotides, between the ends of the first and second probes in order to create the complete functional domain in the chimeric probe; and steps (f)-(i) as described above.

### 3. Ligation Conditions

In some instances, ligation is performed in a ligation buffer. In instances where probe ligation is performed on diribo-containing probes, the ligation buffer can include T4 RNA Ligase Buffer 2, enzyme (e.g., RNL2 ligase), and nuclease free water. In instances where probe ligation is performed on DNA probes, the ligation buffer can include Tris-HCl pH7.5, MnCl2, ATP, DTT, surrogate fluid (e.g., glycerol), enzyme (e.g., SplintR ligase), and nuclease-free water. In some forms, the ligation buffer includes additional reagents. In some instances, the ligation buffer includes adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers the AMP to the phosphorylated 5′-end at the nick, forming a 5′-5′ pyrophosphate bond. Finally, the ligase catalyzes an attack on this pyrophosphate bond by the OH group at 3′-end of the nick, thereby sealing it, whereafter ligase and AMP are released. If the ligase detaches from the substrate before 3′ attack, e.g., because of premature AMP reloading of the enzyme, then 5′ AMP is left at 5′-end, blocking further ligation attempts. In some instances, ATP is added at a concentration of about 1 μM, about 10 μM, about 100 μM, about 1000 μM, or about 10000 μM during the ligation reaction.

After ligation, in some instances, the biological sample is washed with a post-ligation wash buffer. In some instances, the post-ligation wash buffer includes one or more of saline-sodium citrate (SSC; 1×), ethylene carbonate or formamide, and nuclease free water. In some instances, the biological sample is washed at this stage at about 50° C. to about 70° C. In some instances, the biological sample is washed at about 60° C.

### 4. Pre-Adenylated 5′ Phosphate on Probe

In some forms, when the methods include hybridizing a target analyte in the biological sample with an analyte capture domain of a first or second oligonucleotide, and/or with a complementary sequence of a third oligonucleotide, in some forms the first oligonucleotide includes, from 3′ to 5′, a sequence substantially complementary to a first sequence in the third oligonucleotide and a sequence that is substantially complementary to a sequence in the target analyte and has a pre-adenylated phosphate group at its 5′ end; and whereby the second oligonucleotide includes a sequence substantially complementary to a second sequence in the third oligonucleotide. The methods then generate a ligation product by ligating a 3′ end of the first oligonucleotide to 5′ end of the second oligonucleotide using a ligase that does not require adenosine triphosphate for ligase activity. The methods typically further include releasing the ligation product from the third oligonucleotide and binding the ligation product specifically to the capture domain of the immobilized capture probe; and proceeding according to the described methods.

### 5. Streamlined Methods Using a Single Probe

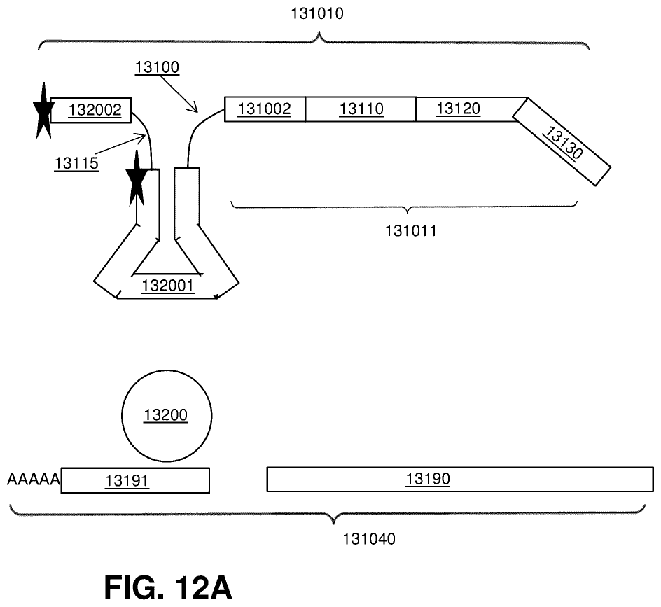

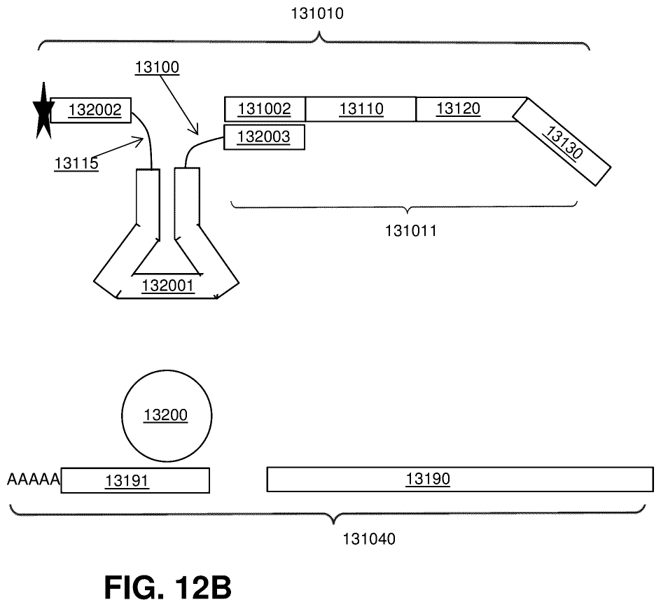

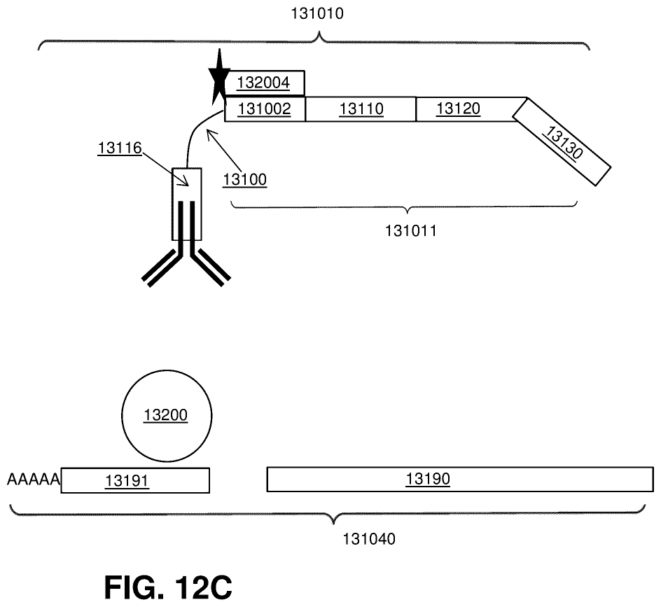

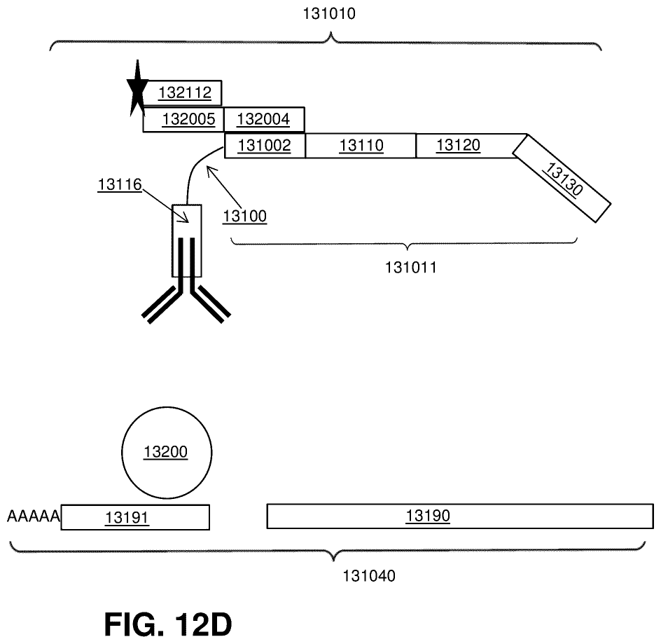

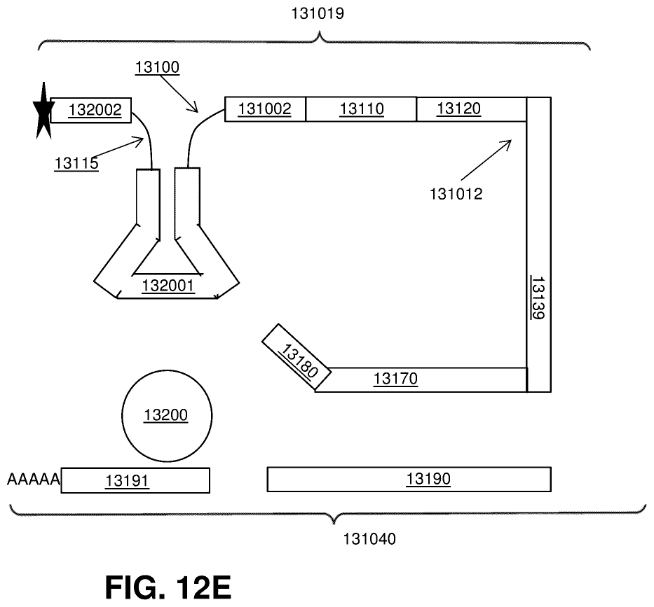

In some forms, the methods combine the function of the first and second probes into a single, functionalized, bi-specific probe that is capable of simultaneously binding to both a target nucleic acid binding protein and to proximal nucleic acid. For example, in an exemplary form, the methods employ a functionalized probe having a region of complementarity to an mRNA and which is also attached to a protein binding moiety. See, for example, FIGS. 12E and 15C. The affinity of the protein binding moiety for a target protein, and/or the affinity of the nucleic acid-binding domain for a target nucleic acid are tuned such that binding will only occur when both the nucleic acid binding domain and the protein binding domain interact with/hybridize to their target ligands. Non-bound probes can be removed in one or more wash steps, prior to embedding the probe within a matrix, such as a gel.

Typically, the distance between the nucleic acid and protein binding moieties associated with the functionalized, bi-specific probe is configured to limit the detection methods to protein/nucleic acid interactions. For example, the size of the functionalized, bi-specific probe can be limited to between 10 and 100 nucleotides. Typically, when the methods employ a single functionalized, bi-specific probe, the methods utilize one or more steps to first bind the functionalized, bi-specific probe to the target protein and then to remove any unbound probes from the biological sample. Exemplary method steps to remove unbound functionalized, bi-specific probes include wash steps. The methods typically include one or more subsequent steps to functionalize the nucleic acids in the sample and then embed the functionalized bi-specific protein-bound probe into a matrix. The methods include one or more subsequent steps to hybridize the functionalized, bi-specific probe to proximal nucleic acids. The timing and efficacy of hybridization can be controlled, for example, by changing one or more parameters, such as temperature. Therefore, in some forms, the methods control the timing of binding to target proteins and hybridizing to target nucleic acids to control and/or regulate probe binding.

In some forms, the target protein is an RNA-binding protein and the target nucleic acid is an RNA, such as mRNA. In some forms, when the target nucleic acid is mRNA, the bi-specific probe includes a binding site that can bind to any RNA, for example, by including a region of complementarity to any mRNA. An exemplary region of complementarity to any RNA incudes a poly-(T) region that can hybridize to the poly-(A) region of all mRNAs. In other forms, the bi-specific probe includes a binding site that can bind to a specific mRNA, or a specific group of mRNAs, for example, by including a region of complementarity to one or more coding regions of an mRNA transcript that is specific to one or more gene transcription products.

Typically, the single bi-specific probe includes a capture domain binding sequence that permits capture of the oligonucleotide to a capture domain on a capture probe having a spatial barcode. Generally, the step of capturing a bi-specific probe on a capture probe includes hybridizing the capture domain binding sequence to the capture domain. To prevent premature or non-specific binding to the capture probe, the methods can employ blocking probes to block or otherwise prevent premature binding to the capture probe. In some forms, the methods prevent premature binding to the capture probe by altering the temperature and/or other conditions to limit or prevent hybridization of the capture domain binding sequence to the capture domain of a capture probe until desired. Suitable conditions and buffers for washing unbound protein binding moieties away from the sample, as well as hybridization conditions are described herein.

In some forms, methods of determining a location and/or abundance of an interaction between a target nucleic acid binding protein and a nucleic acid, each endogenous to a subject, in a biological sample obtained from the subject, include:
(a) contacting a biological sample including a plurality of nucleic acids with a plurality of bi-specific probe. Each bi-specific probe typically includes at least the components (i), (ii) and (iii), as follows: (i) a target protein-binding moiety and (ii) an associated first oligonucleotide including a region of complementarity to a nucleic acid and, optionally, a target protein identification barcode, and (iii) one or more functional group(s) for gel embedding, and (iv) a capture domain binding sequence. Typically, the contacting is carried out under conditions suitable for the target protein-binding moiety to bind to a target protein only when the nucleic acid binding domain is simultaneously bound to a target nucleic acid.
(b) washing the sample to remove non-bound probes.
(c) modifying a 3′ and/or 5′ end of the nucleic acids in the sample with one or more functional group(s) for embedding within a permeable matrix, such as a permeable gel or hydrogel.
(d) embedding the biological sample in a permeable matrix, such as a permeable gel or hydrogel. The process of embedding generally includes forming an interaction between the one or more functional group(s) and the matrix.
(e) hybridizing the plurality of immobilized/embedded bi-specific probe to nucleic acids in the region of the target protein;
(f) releasing the bi-specific probe from the non-nucleic acid components sample. Exemplary non-nucleic acid components include proteins, carbohydrates, lipids, and combinations thereof. In some forms, the step of releasing removes dyes, labels, small molecules, etc., from the embedded sample.
(g) hybridizing the capture domain binding sequence of the bi-specific probe to the capture domain of a capture probe, such as an arrayed capture probe. Each second probe typically includes at least the components (i) and (ii), as follows: (i) a spatial barcode; and (ii) a capture domain;
(h) determining one or more of (hi) the spatial barcode sequence or a complement thereof; (hii) all or a portion of the bi-specific probe sequence or a complement thereof; and/or (hiii) the sequence of the target protein identification barcode; and
(i) using the determined sequences of (hi), and/or (hii) and/or (hiii) to identify the location and/or abundance of the interaction between the target protein and nucleic acid in the biological sample.

### B. Nucleic Acid-Binding Protein Interactions

In some forms, the methods determine a location and/or abundance of an interaction between a target nucleic acid binding protein and a nucleic acid in a biological sample. In some forms, the nucleic acid binding protein is a DNA binding protein. In some forms the nucleic acid binding protein is an RNA binding protein.

### 1. RNA Binding Proteins (RBPs)

Methods for enhanced spatial analysis of RNAs interacting with RNA binding proteins are described.

RNA-binding proteins (RBPs) are an important type of intracellular protein, that can be widely involved in a variety of post-transcriptional regulation processes, such as RNA splicing, transport, localization, and translation. RBPs can be divided by function, including Human antigen R (HuR), heterogeneous nuclear ribonucleoprotein family (hnRNP), the arginine/serine-rich splicing factor protein family (SRSF), and RNA-binding motif (RBM) proteins. In some forms the methods determine a location and/or abundance of an interaction between RNA and a target nucleic acid binding protein selected from Human antigen R (HuR), heterogeneous nuclear ribonucleoprotein family (hnRNP), the arginine/serine-rich splicing factor protein family (SRSF), and RNA-binding motif (RBM) proteins.

RNA-binding proteins (RBPs) play an important role in governing the fate of mRNA transcripts from biogenesis, stabilization, translation to RNA decay. Therefore, in some forms, the methods determine a location and/or abundance of an interaction between RNA and a target nucleic acid binding protein that performs one or more functions selected from mRNA biogenesis, mRNA stabilization, mRNA translation and mRNA decay.

Characterization of transcriptional and genomic alterations in the landscape of RNA-binding proteins has identified multiple disease-related RBPs that showed altered expression and/or activity in tumors as compared to normal tissues. RBPs are ubiquitously expressed and evolutionarily conserved and exhibit higher expression than other protein-coding genes. RBP expression is tightly controlled and Dysregulated expression of RBPs can lead to imbalanced cellular homeostasis and diseases, including cancer. Overexpression of the mRNA 5′ cap-binding protein eIF4E promotes malignant transformation in human and mouse cells and tumor growth is inhibited by administering antisense oligonucleotides (ASOs) against eIF4E. Therefore, determining expression profiles of RBPs in normal and cancer tissues provides means for early detection and monitoring of tumorigenesis and cancer progression. In some forms, the methods determine a location and/or abundance of an interaction between RNA and a target nucleic acid binding protein that is associated with a disease or disorder, such as cancer.

Therefore, in some forms, the methods determine a location and/or abundance of an interaction between a target mRNA-binding protein and mRNA, each endogenous to a subject, in a biological sample obtained from the subject.

### (a.) Varied Expression of RBPs

In some forms, the methods determine and measure differences in the abundance of an interaction between a target RNA binding protein and RNA, each endogenous to a subject, in one or more biological samples obtained from a subject.

The cancer genome is characterized by copy number alterations and it often carries a large number of somatic mutations, including base substitutions, small insertions and deletions (indels), and rearrangements. These aberrations can promote tumorigenesis, and many such cancer “driver genes” are RBPs, including DICER1 (an RBP that is responsible for microRNA (miRNA) maturation, which is mutated in a diverse array of cancer types and plays a tumor suppressive role) and splicing factor SF2/ASF (upregulated in multiple tumors due to gene amplification and can promote tumorigenesis by regulating the alternative splicing of the tumor suppressor gene BIN1 and the kinases MNK2 and S6K1).

Studies to characterize differentially expressed RBPs between tumors and matched normal tissues have identified multiple differentially-expressed human RBPs that are specific to cancer types (Wang, et al, Cell Reports, v22 (1) pp. 286-298, (2018)). Therefore, in some forms, the methods identify the amount and/or spatial distribution of RBPs in a sample from a subject as compared to a healthy control subject, where a change in the somatic copy number (somatic copy number alteration (SCNA)) of the RBP in the sample from a subject as compared to a healthy control subject informs the presence of a potential tumor in the subject. In some forms, the methods identify the amount and/or spatial distribution of RBPs (and associated nucleic acids) in a first region of a tissue sample from a subject as compared to a second region of the tissue sample. In such forms, a change in the somatic copy number (somatic copy number alteration (SCNA)) of the RBP in the first region of the tissue sample as compared to the second region can inform the presence of a potential tumor or cancer in a particular region of the tissue sample.

(i.) Exemplary RBP Genes Associated with Human Diseases

In some forms, the target nucleic acid binding protein is an expression product of a gene associated with a human disease that exhibits altered expression and/or copy number in diseased tissue as compared with healthy tissue. Exemplary genes coding for RBPs that exhibit altered expression and copy number in tumor tissue as compared with healthy tissue include, but are not limited to BYSL, ZC3H13, ELAC1, RBMS3, NSUN6, ZGPAT, TERT, ZC3H12C, ZNF106, TLR3, PIWIL2, APC, EXO1, RNASE4, SRSF12, RBM11, LRRFIP1, OASL, SMAD4, DKC1, ANG, SRSF5, KHDRBS2, ARHGEF28, SIDT2, RBPMS, INTS10, SECISBP2L, FTO, EIF1B, RNASE1, DICER1, EZH2, LARS2, ZCCHC2, ZFP36, EXOSC7, NOVA1, MEX3A, HINT3, RBMS3, RNASE6, PATL2, BYSL, ESRP2, CNOT8, RPL22L1, POLR2H, RNASE3, ELAVL2, MRPS36, PPARGC1B, ADAD2, CPEB4, PTBP1, CTIF, HBS1L, PNRC2, MRPL12, TDRD9, DYNC1H1, ADARB2, PDCD4, TEP1, DDX24, GFM1, TARBP1, ELAC1, RPL22, PTBP2, TARS, RBM7, BRIX1, ENOX1, SRRM4, CWF19L2, ZFP36L1, CLK4, DUS1L, HABP4, METTL1, ELAVL1, TIAR1 and XPO4. The difference in expression can be an increase or a decrease in expression of the gene product. In preferred forms, the genes coding for RBPs that exhibit altered expression and copy number in tumor tissue as compared with healthy tissue include are selected from the gene products of BYSL, ZC3H13, ELAC1, RBMS3, NSUN6, and ZGPAT.

In an exemplary form, the gene product of BYSL, a protein linked to embryo implantation and ribosomal processing of 18S rRNA and nucleolar assembly, is overexpressed in colorectal cancer. Therefore, in an exemplary method, the identification of an increase in the abundance of interactions between RNA and the BYSL gene product in a sample from the colon or rectum of a subject as compared with that in a control sample from a healthy subject is indicative of a colorectal tumor in the subject.

In an exemplary form the gene product of NSUN6, a m5C tRNA methyltransferase that mainly acts on substrates tRNACys and tRNAThr, is downregulated in Liver Hepatocellular Carcinoma (LIHC). Therefore, in an exemplary method, the identification of an increase in the abundance of interactions between RNA and the NSUN6 gene product in a sample from the liver of a subject as compared with that in a control sample from a healthy subject is indicative of a hepatocellular carcinoma in the subject.

### (b.) Altered RBP/mRNA Interactions

In some forms, the methods detect and/or characterize altered or abnormal RBP/mRNA interactions in a biological sample.

Abnormal post-transcriptional regulation induced by alterations of mRNA-protein interactions is critical during tumorigenesis and cancer progression, and is a hallmark of cancer cells. Studies investigating post-transcriptional regulation in cancer on a high throughput scale have identified mRNA-protein interactions that inform the presence and stage of cancer (Blanchard, et al., Cancer Res; 79:5418-31 (2019)).

Therefore, in some forms, the described methods quantify mRNA interactions with RNA-binding proteins relevant for tumorigenesis and cancer progression in a sample from a subject, as compared with those in archival patient derived tumor tissue can inform cancer status in the subject.

After transcription, processing, and transport, mRNA translation and stability is controlled by miRNAs and RBPs in a time- and space-dependent manner (Wurth, Comp Funct Genomics, 178525 (2012)).

(i.) Human antigen R (HuR)

In some forms, the target RBP is Human antigen R (HuR; also known as Elavl1). HuR is a universally expressed RBP encoded by the ELAVL1 gene located on chromosome 19p13.2, which participates in posttranscriptional control of RNAs, such as splicing, polyadenylation, mRNA stabilization, localization and translation.

HuR encodes a 32 kD protein composed of three RNA-binding domains that belong to the RRM family; RRM1, RRM2 and RRM3. HuR regulation of target mRNAs is based on the interaction between the three specific domains of HuR protein and one or several U- or AU-rich elements (AREs) in the untranslated region of target mRNAs and enhances mRNA stability and translation often by competing for AREs occupancy against mRNA-destabilizing modulators. RRM-1 and RRM-2 are responsible for AU rich elements (ARE) binding, and it is RRM-3 that binds to the mRNA poly(A) tail.

HuR is important for development and function of several cell types, and HuR homozygous knockout mice are lethal. HuR activity and function is associated with its subcellular distribution, transcriptional regulation, translational and post-translational modifications: it has been established that HuR is an important posttranscriptional regulator of adipogenesis and coordinates posttranscriptional processes contribute to adipocyte development (Siang, et al. Nat Commun 11, 213 (2020)); and analysis of HuR:RNA interactions showed that HuR was bound to U-rich elements present in 3′UTR of Myc in both resting and mitogen-activated B cells (Osma-Garcia, Nat Commun 12, 6556 (2021)).

(a) HuR mRNA Targets

In some forms, the methods detect and measure the presence, abundance and/or location of interactions between the RBP HuR protein and one or more mRNAs that are known to interact with HuR and have been associated with a disease state. Several specific mRNA targets that are known to interact with HuR are associated with diseases in humans (See, e.g., Srikantan, et al., Front Biosci (Landmark Ed).; 17 (1): 189-205 (2012), the contents of which are incorporated by reference herein in their entirety).

Exemplary mRNAs that are known to bind to HuR and are associated with one or more human diseases or disorders include c-Fos, c-Myc, p21, p27, cyclin A2, cyclin B1, cyclin E1, cyclin D1, OSM, eIF4E, EGF, VEGF, HIF-1α, COX-2, iNOS, TSP1, TGF-β, MKP-1, Mdm2, SIRT1, Bcl-2, Mcl-1, XIAP, Cyto c, uPA, uPAR, MMP-9, Snail, dCK, p53, pVHL, ARH1/DRAS3, BRCA1, Estrogen receptor (ER), Wnt5a, c-Fms, GATA3, GM-CSF, TNF-α, TM, RGS4, TLR4, IL-6, IL-8, IL-13, SMN, SH2D1A, NF1, PROX1, Eotaxin, ProTα, and IGF-1RA. A summary of the specific mRNAs known to interact with HuR, the influence of HuR and the associated disease states is provided in Table 1. Therefore, in some forms, the methods detect and measure the presence, abundance and location of interactions between the RBP HuR protein and one or more mRNAs set forth in Table 1.

TABLE 1
mRNA Targets of HuR and associated Disease States

| HuR | Influence | | |
|---|---|---|---|
| target | of HuR on | Processes | |
| mRNA | mRNA | Regulated | Disease State |
| c-Fos | ↑ Stability | Proliferation | Oral squamous |
| | | | carcinoma |
| c-Myc | ↓ Translation | Proliferation, | Cervical carcinoma |
| | | survival | |
| p21 | ↑ Stability | Proliferation, | Carcinoma (breast, |
| | | survival | colon) |
| p27 | ↓ Translation | Proliferation, | Cervical carcinoma |
| | | survival | |
| cyclin A2 | ↑ Stability | Proliferation | Carcinoma (colon, |
| | ↑ Translation | | gastric, oral) |
| cyclin B1 | ↑ Stability | Proliferation | Oral carcinoma |
| cyclin E1 | ↑ Stability | Proliferation | Breast carcinoma |
| cyclin D1 | ↑ Stability | Proliferation | Carcinoma (oral, colon) |
| OSM | ↑ Stability | Proliferation | Lymphoma |
| eIF4E | ↑ Stability | Proliferation, | Pharyngeal carcinoma |
| | | survival | |
| EGF | ↑ Stability | Proliferation | Prostate carcinoma |
| VEGF | ↑ Stability | Angiogenesis, | Carcinoma (colon, non- |
| | ↑ Translation | proliferation | small cell lung, kidney, |
| | | | pancreatic, prostate), |
| | | | glioma, meningioma, |
| | | | astrocytoma, ischemia, |
| | | | amyotrophic lateral |
| | | | sclerosis) |
| HIF-1α | ↑ Stability | Angiogenesis, | Cervical carcinoma, |
| | ↑ Translation | survival | ischemia |
| COX-2 | ↑ Stability | Angiogenesis, | Carcinoma (colon, |
| | | survival, | ovarian, gastric, oral, |
| | | inflammation | prostate), central nervous |
| | | | system malignancies, |
| | | | rheumatoid cartilage, |
| | | | osteoarthritic cartilage, |
| | | | inflammatory, bowel |
| | | | disease |
| iNOS | ↑ Stability | Angiogenesis, | Colon carcinoma, muscle |
| | | survival, | wasting |
| | | inflammation | |
| TSP1* | ↑ Translation | Angiogenesis | Breast carcinoma |
| TGF-β | ↑ (n.d.) | Immunity, | Tumors of the central |
| | | inflammation | nervous system |
| MKP-1 | ↑ Stability | Signaling, | Cervical carcinoma |
| | ↑ Translation | immunity | |
| Mdm2 | ↑ Stability | Survival | Intestinal epithelium |
| | | | function |
| SIRT1 | ↑ Stability | Survival, stem | Carcinoma (cervical, |
| | | cell development | prostate) |
| Bcl-2 | ↑ Stability | Survival | Carcinoma (cervical, |
| | ↑ Translation | | prostate, epidermoid), |
| | | | leukemia, ischemia- |
| | | | reperfusion injury |
| Mcl-1 | ↑ (n.d.) | Survival | Cervical carcinoma |
| XIAP | ↑ Translation | Survival | Untransformed cells |
| Cyto c | ↑ Translation | Survival | Cervical carcinoma |
| uPA | ↑ Stability | Invasion, | Breast carcinoma |
| | | migration | |
| uPAR | ↑ Stability | Invasion, | Breast carcinoma |
| | | migration | |
| MMP-9 | ↑ Stability | Invasion | Fibrosarcoma, myeloid |
| | | | leukemia, fibrosis, left |
| | | | ventricular function and |
| | | | remodeling |
| Snail | ↑ Stability | Invasion | Breast carcinoma |
| dCK | ↑ (n.d.) | Chemotherapy | Pancreatic carcinoma |
| ARHI/ | ↑ Stability | Tumor | Ovarian carcinoma |
| DRAS3* | | suppression | |
| p53 | ↑ Stability | Tumor | Carcinoma (cervical, |
| | ↑ Translation | suppression | gastric, liver, colon), |
| | | | intestinal epithelium |
| | | | function, myocardial |
| | | | infarction |
| pVHL | ↑ Stability | Tumor | VHL syndrome, kidney |
| | | suppression | carcinoma |
| BRCA1 | (↓) n.d. | Tumor | Breast carcinoma |
| | | suppression | |
| ER | ↑ Stability | Tumorigenesis | Breast carcinoma |
| Wnt5a | ↓ Translation | Tumorigenesis | Breast carcinoma |
| c-Fms | ↑ Stability | Tumorigenesis | Breast carcinoma |
| GATA3 | ↑ Stability | Tumorigenesis | Breast carcinoma |
| GM-CSF | ↑ Stability | Inflammation, | Asthma, T cell |
| | | immunity | activation, atherogenesis |
| TNF-α | ↑ Stability | Inflammation, | Muscle wasting, |
| | | immunity | malignant glioma, |
| | | | rheumatoid arthritis, |
| | | | atherosclerosis |
| TM | ↓ Translation | Inflammation | Sepsis |
| RGS4 | ↑ Stability | Inflammation | Smooth muscle |
| | | | contraction, cardiac |
| | | | development |
| TLR4 | ↑ Stability | Inflammation, | Vascular smooth muscle |
| | | immunity | hyperplasia |
| IL-6 | ↑ Stability | Inflammation, | Tumors of the central |
| | | immunity | nervous system, viral |
| | | | infection, atherosclerosis |
| IL-8 | ↑ Stability | Inflammation, | Carcinoma (breast, |
| | | immunity | colon, gastric), glioma |
| IL-13 | ↑ Stability | Inflammation | Allergy |
| SMN | ↑ Stability | Neuropathology | Spinal muscle atrophy |
| SH2D1A | ↑ Stability | Proliferation | X-linked |
| | | | lymphoproliferative |
| | | | disease |
| NF1 | ↑ Stability | Signaling | Neurofibromatosis |
| PROX1 | ↑ Stability | Endothelial | Kaposi's sarcoma |
| | | differentiation | |
| Eotaxin | ↑ Stability | Inflammation | Asthma |
| ProTα | ↑ Translation | Tumorigenesis | Cervical carcinoma |
| IGF-1R | ↓ Translation | Proliferation | Breast carcinoma |
| HuR | ↑ Stability | (above processes) | (above cell/disease |
| | ↑ Translation | | models) |

(b) HuR/mRNA Interactions Associated with Cancer

In particular forms, the methods detect and measure the presence, abundance and/or location of interactions between the RBP HuR protein and one or more mRNAs that are known to interact with HuR and have been associated with cancer. It has been shown that HuR undergoes overexpression and cytoplasmic translocation in tumor tissue from multiple types of cancers.

HuR has a central tumorigenic activity by enabling multiple cancer phenotypes, and a number of cancer-related transcripts containing U- or AU-rich elements (AREs), including mRNAs for proto-oncogenes, cytokines, growth factors, and invasion factors, have been characterized as HuR targets. The expression of HuR and other RBPs are altered in several human cancers, such as breast cancer, lung cancer, mesothelioma, ovarian cancer and colon cancer. Increased expression of HuR occurs in virtually all cancer tissues compared to the normal-tissue counterparts and collections of HuR-regulated mRNAs were identified in colon cancer cells by cDNA arrays. Increased HuR expression and cytoplasmic localization are found in 76% of adenomas and 94% of adenocarcinomas. Only low levels of HuR are present in normal colon tissues (López de Silanes, et al., Nucleic Acids Res, 37(8):2658-71 (2009)).

HuR interacts with and regulates many mRNAs encoding cancer-related proteins. HuR is upregulated in virtually all cancer types, and has been proposed to coordinate the expression of cancer genes and thereby impact upon phenotypic traits central to tumorigenesis. In breast carcinomas, elevated cytoplasmic HuR levels were associated with tumor grade and poor patient outcome. In breast cancer cells, HuR increased expression of cyclin E1, IL-8, estrogen receptor, TSP1, and c-Fms, while HuR repressed the translation of Wnt5a, a protein that inhibits tumor growth. In pancreatic cancer, high HuR levels correlated with high levels of VEGF and with poor patient prognosis.

Increased HuR levels are associated with high ovarian tumor grade and poor prognosis. In prostate cancers, HuR abundance is linked to increased levels of COX-2, prostate-specific antigen (PSA), SIRT1, and EGF. In keeping with the view that cytoplasmic HuR promotes prostate tumor development and relapse, patients who had elevated cytoplasmic HuR expressed higher COX-2 and adverse prognosis with shorter disease-free survival times. HuR is also upregulated in oral, lung, gastric, and pharyngeal carcinomas, as well as in cancers of the central nervous system (e.g., meningioma, glioma, astrocytoma), where COX-2, c-Fos, VEGF, eIF4E, cyclin D1, cyclin A, and other HuR target mRNAs were found to be elevated.

### (c) HuR Binding Agents

In some forms, the methods detect and measure the presence, abundance and/or location of interactions between HuR and a bound ligand.

Multiple HuR-specific binding agents are known in the art and are available from multiple commercial sources, including monoclonal antibodies specific for the HuR protein and antigen-binding fragments thereof. Exemplary antibodies include rabbit monoclonal clone #EPR17397 (Abcam Cat #ab200342), mouse monoclonal clone #19F12AE12 (Abcam Cat #ab170193), and mouse monoclonal clone #4C8 (Abcam Cat #ab136542). Any of these binding moieties can be included as target-specific HuR capture agents in the described methods.

### (d) HuR and COX2

In some forms, the methods detect and measure the presence, abundance and/or location of interactions between HuR and COX2.

An important carcinogenesis related factor is cyclooygenase-2 (COX-2). This protein is an inducible enzyme critically involved in the synthesis of prostaglandins. The prostaglandins have been widely studied because HuR regulates their abnormal expression, especially in gastric and colorectal carcinoma. Overexpression of both HuR and COX-2 has been observed in many cancer types, including colon and lung cancers: high abundance of HuR in colon cancer contributes to the increased expression of COX-2 and VEGF levels and is associated with advanced tumor stage. Overexpression of HuR increased the growth of colon cancer cells and COX-2 levels are upregulated in ovarian carcinomas, where both nuclear and cytoplasmic HuR are elevated.

Therefore, in some forms, the methods detect and measure the presence, abundance and location of interactions between the RBP HuR protein and COX-2 mRNA.

### (ii.) RNA-Binding Motif (RBM) Proteins

In some forms, the methods detect and measure the presence, abundance and/or location of interactions between an RNA-Binding Motif (RBM) Protein and one or more mRNAs that are known to interact with the RBM protein.

RNA-binding motif (RBM) proteins are a class of RNA-binding proteins containing RNA-recognition motifs (RRMs), RNA-binding domains, and ribonucleoprotein motifs. RBM proteins are involved in RNA metabolism, including splicing, transport, translation, and stability. Many studies have found that aberrant expression and dysregulated function of RBM proteins family members are closely related to the occurrence and development of cancers. RBM protein family genes are known to be associated with cancers, including cancer occurrence and cell proliferation, migration, and apoptosis (See, e.g., Wu, et al., Adv Drug Deliv Rev., 184:114179 (2022), the content of which is hereby incorporated by reference herein in its entirety).

Exemplary genes coding for RBMs that are associated with human disease and disorders include, but are not limited to RBM3, RBM4, RBM5, RBM6, RBM7, RBM8A, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM33, RBM34, RBM38, RBM39, RBM41, RBM42, RBM43, RBM44, RBM45, RBM46, RBM47, RBM48, RBM4B, RBMS1, RBMS2, RBMS3, RBMX, RBMX2, RBMXL1, RBMXL2, RBMXL3, RBMY1A1, RBMY1B, RBMY1C, RBMY1D, RBMY1E, RBMY1F, and RBMY1J.

Exemplary RBMs that are associated with human disease and disorders include, but are not limited to RNA Binding Motif Protein 3 RNA Binding Motif Protein 3, RNA Binding Motif Protein 4, RNA Binding Motif Protein 5

RBM5 Antisense RNA 1, RNA Binding Motif Protein 6, RNA Binding Motif Protein 7, RNA Binding Motif Protein 8A, RNA Binding Motif Protein 10, RNA Binding Motif Protein 11, RNA Binding Motif Protein 12, RNA Binding Motif Protein 12B, RNA Binding Motif Protein 14, RBM14-RBM4 Readthrough, RNA Binding Motif Protein 15, RNA Binding Motif Protein 15B, RNA Binding Motif Protein 17, RNA Binding Motif Protein 18, RNA Binding Motif Protein 19, RNA Binding Motif Protein 20, RNA Binding Motif Protein 22, RNA Binding Motif Protein 23, RNA Binding Motif Protein 24, RNA Binding Motif Protein 25, RNA Binding Motif Protein 26, RNA Binding Motif Protein 27, RNA Binding Motif Protein 28, RNA Binding Motif Protein 33, RNA Binding Motif Protein 34, RNA Binding Motif Protein 38, RNA Binding Motif Protein 39, RNA Binding Motif Protein 41, RNA Binding Motif Protein 42, RNA Binding Motif Protein 43, RNA Binding Motif Protein 44, RNA Binding Motif Protein 45, RNA Binding Motif Protein 46, RNA Binding Motif Protein 47, RNA Binding Motif Protein 48, RNA Binding Motif Protein 4B, RNA Binding Motif Single Stranded Interacting Protein 1, RNA Binding Motif Single Stranded Interacting Protein 2, RNA Binding Motif Single Stranded Interacting Protein 3, RNA Binding Motif Protein X-Linked, RNA Binding Motif Protein X-Linked 2, RBMX Like 1, RBMX Like 2, RBMX Like 3, RNA Binding Motif Protein Y-Linked Family 1 Member A1, RNA Binding Motif Protein Y-Linked Family 1 Member B, RNA Binding Motif Protein Y-Linked Family 1 Member C, RNA Binding Motif Protein Y-Linked Family 1 Member D, RNA Binding Motif Protein Y-Linked Family 1 Member E, RNA Binding Motif Protein Y-Linked Family 1 Member F, and RNA Binding Motif Protein Y-Linked Family 1 Member J. Exemplary diseases that are known to be associated with specific RBM proteins are set forth in Table 2. Therefore, in some forms, the methods detect and measure the presence, abundance and location of interactions between RNA and one of the RBM protein set forth in Table 2.

TABLE 2
Diseases associated with aberrant expression of RBM proteins

| RBM Protein | Associated Disease |
|---|---|
| RNA Binding Motif Protein 3 | Testicular Malignant Germ Cell Cancer; Noonan Syndrome 6, |
| | Breast cancer, Colorectal cancer, Hepatocellular carcinoma, |
| | Pancreatic cancer, Prostate cancer. |
| RNA Binding Motif Protein 4 | Down Syndrome. |
| RNA Binding Motif Protein 5 | Lung Cancer. |
| RBM5 Antisense RNA 1 | Glioblastoma and Colorectal Cancer, Hepatocellular |
| | Carcinoma, Osteosarcoma, Oral squamous cell carcinoma |
| RNA Binding Motif Protein 6 | Testis Seminoma and Lung Cancer. |
| RNA Binding Motif Protein 7 | Pontocerebellar Hypoplasia, Breast cancer |
| RNA Binding Motif Protein 8A | Thrombocytopenia-Absent Radius Syndrome and Thrombocytopenia. |
| RNA Binding Motif Protein 10 | Tarp Syndrome and Skin Angiosarcoma, Lung cancer. |
| RNA Binding Motif Protein 11 | Ovarian cancer |
| RNA Binding Motif Protein 12 | Schizophrenia 19 and Schizoaffective Disorder |
| RNA Binding Motif Protein 12B | Primary Cerebellar Degeneration and Boucher-Neuhauser |
| | Syndrome. |
| RNA Binding Motif Protein 14 | Spinal Muscular Atrophy With Progressive Myoclonic Epilepsy. |
| RBM14-RBM4 Readthrough | Spinal Muscular Atrophy With Progressive Myoclonic Epilepsy |
| | and Farber Lipogranulomatosis. |
| RNA Binding Motif Protein 15 | Megakaryoblastic Acute Myeloid Leukemia With |
| | T(1; 22)(P13; Q13) and Acute Megakaryocytic Leukemia, |
| | Laryngeal squamous cell carcinoma |
| RNA Binding Motif Protein 15B | Severe Congenital Neutropenia 1 and Immunodeficiency With |
| | Hyper-Igm, Type 5. |
| RNA Binding Motif Protein 17 | Spinocerebellar Ataxia 1 and Dentatorubral-Pallidoluysian |
| | Atrophy, Glioma, Hepatocellular carcinoma, Hypopharyngeal |
| | carcinoma |
| RNA Binding Motif Protein 19 | Autosomal Dominant Non-Syndromic Intellectual Disability |
| | 1 and Ulnar-Mammary Syndrome |
| RNA Binding Motif Protein 20 | Cardiomyopathy, Dilated, 1Dd and Dilated Cardiomyopathy. |
| RNA Binding Motif Protein 23 | Hepatocellular carcinoma |
| RNA Binding Motif Protein 24 | Atrial Septal Defect 2 and Neuropathy, Hereditary Sensory And |
| | Autonomic, Type Iii and Bladder cancer |
| RNA Binding Motif Protein 25 | Atrial Septal Defect 9 and Brugada Syndrome. |
| RNA Binding Motif Protein 26 | Cutaneous T Cell Lymphoma and Helsmoortel-Van Der Aa |
| | Syndrome. |
| RNA Binding Motif Protein 27 | Mucocutaneous Leishmaniasis and Failure Of Tooth Eruption, |
| | Primary. |
| RNA Binding Motif Protein 28 | Alopecia, Neurologic Defects, And Endocrinopathy |
| | Syndrome and Alopecia. |
| RNA Binding Motif Protein 33 | Acheiropody and Tibia, Hypoplasia Or Aplasia Of, With |
| | Polydactyly, Gastric cancer, Cervical cancer |
| RNA Binding Motif Protein 38 | Lung Cancer and Erythema Infectiosum. |
| RNA Binding Motif Protein 39 | Hepatocellular Carcinoma. Among its related pathways |
| | are mRNA Splicing - Major Pathway, Breast cancer |
| RNA Binding Motif Protein 45 | Tick Infestation and Amyotrophic Lateral Sclerosis 1 |
| RNA Binding Motif Protein 46 | Basal Cell Nevus Syndrome. |
| RNA Binding Motif Protein 47 | Lung Acinar Adenocarcinoma, Nasopharyngeal carcinoma |
| RNA Binding Motif Protein 48 | Nephronophthisis and Peroxisome Biogenesis Disorder 1B. |
| RNA Binding Motif Protein 4B | Episodic Ataxia, Type 6. Among its related pathways are |
| | Circadian rythm related genes. |
| RNA Binding Motif Single | Diffuse Glomerulonephritis and Arthrogryposis Multiplex |
| Stranded Interacting Protein 1 | Congenita 2, Neurogenic Type. |
| RNA Binding Motif Single | Variola Minor and Hemolytic Uremic Syndrome, Atypical 1. |
| Stranded Interacting Protein 2 | |
| RNA Binding Motif Single | Mental Retardation With Language Impairment And With Or |
| Stranded Interacting Protein 3 | Without Autistic Features and Heimler Syndrome 2. |
| RNA Binding Motif Protein X- | Syndromic X-Linked Intellectual Disability Shashi Type and X- |
| Linked | Linked Hereditary Ataxia, Hepatocellular Carcinoma, |
| | Myeloid leukemia |
| RNA Binding Motif Protein X- | Theileriasis and Spermatogenic Failure, X-Linked, 1. |
| Linked 2 | |
| RBMX Like 1 | Autosomal Recessive Nonsyndromic Deafness 32 and Deafness, |
| | Autosomal Recessive 1A. |
| RBMX Like 2 | Cardiomyopathy, Dilated, 3B and Male Infertility. |
| RBMX Like 3 | Cardiomyopathy, Familial Restrictive, 3 and Retinitis Pigmentosa 38. |
| RNA Binding Motif Protein Y- | Spermatogenic Failure, Y-Linked, 2 and Partial Deletion Of Y. |
| Linked Family 1 Member A1 | |
| RNA Binding Motif Protein Y- | RBMY1C is a Protein Coding gene. Gene Ontology (GO) annotations |
| Linked Family 1 Member C | related to this gene include RNA binding and nucleotide binding. |

### C. Functionalizing and Embedding

The methods for spatial analysis of analyte-analyte interactions described herein include one or more of the following steps for embedding the probe(s) and/or nucleic acids in a biological sample within a suitable permeable matrix (e.g., a gel, such as a hydrogel).

The nucleic acid can be modified to include and/or make available one or more functional group(s). An exemplary functional group(s) is acrydite.

An exemplary functional group(s) is a 5′ phosphate or a 5′ phosphate modified with a leaving group.

Provided herein are methods for analyzing a target nucleic acid (e.g., RNA) in a biological sample (e.g., a tissue sample), e.g., wherein the RNA is immobilized according to any of the methods disclosed herein. For examples, disclosed herein are methods of determining a location and/or abundance of an interaction between a target protein and a target nucleic acid (e.g., RNA) in a biological sample. The methods can also involve direct immobilization of any RNA analyte, whether fragmented or not, wherein the RNA analyte possesses a 5′-phosphate group. In some forms, the RNA is not fragmented RNA. The methods can include a series of enzymatic and non-enzymatic reactions that can be utilized to immobilize or tether a nucleic acid (e.g., RNA) to an endogenous molecule in the biological sample or an exogenous molecule delivered to the biological sample, such as a matrix-forming agent. More specifically, the methods provided herein permit tethering 5′ end of RNA to a three-dimensional matrix, such as hydrogel. In some forms, a ribonucleic acid having a fragmented terminal 5′ end is converted to RNA comprising a 5′-phosphate group. In some forms, the 5′-phosphate group moiety is further linked to an attachment agent that allows the RNA to bind covalently or bind non-covalently to a matrix forming agent.

In one aspect, the methods for embedding the biological sample and/or nucleic acids included therein, include: (a) providing a biological sample including a ribonucleic acid (RNA) including a 5′-phosphate group, or a 5′-phosphate group modified with a leaving group; (b) contacting the biological sample with an attachment agent; and (c) forming a covalent bond between the reactive moiety of the attachment agent and the RNA. In some forms, the methods further include: (d) contacting the biological sample with a matrix forming agent. In some forms, the methods further include (e): forming a three-dimensional polymerized matrix from the matrix-forming agent, thereby embedding the biological sample and immobilizing the RNA in the three-dimensional polymerized matrix. In some forms, step (e) is after step (d), and step (d) is after step (c).

The disclosed methods are especially suitable for immobilization of fragmented RNA. In some forms, any one of the RNA analytes disclosed herein can be fragmented. Thus, in some forms, the RNA is a fragmented RNA. In some forms, the fragmented RNA is fragmented mRNA. In some forms, the fragmented RNA includes a 5′-phosphate group or a 5′-phosphate group modified with a leaving group. In some forms, the 5′-phosphate group or 5′-phosphate group modified with a leaving group is a fragmented 5′ end of the RNA.

In some forms, the method further includes converting 5′ end group of least one RNA in the biological sample into a 5′-phosphate group. In some forms, the method further includes reacting at least one RNA in the biological sample with a polynucleotide kinase to provide the RNA comprising a 5′-phosphate group. In some forms, the polynucleotide kinase comprises a T4 Polynucleotide Kinase (T4 PNK). In some forms, the polynucleotide kinase includes a T7 Polynucleotide Kinase (T7-PNK). In some forms, the polynucleotide kinase is a T4 PNK or a T7-PNK. In some forms, the polynucleotide kinase is T4 PNK. In some forms, the polynucleotide kinase is T7 PNK. In some forms, the biological sample includes fragmented RNA. In some forms, the method includes converting 5′ end groups (e.g., 5′-OH of fragmented RNAs) of least a plurality of RNAs in the biological sample into 5′-phosphate groups.

In some forms, the resulting 5′-phosphate group is further modified with a leaving group, and thereby forming an RNA including a 5′-phosphate group modified with a leaving group. In some forms, the modification involves replacing one-OH group in the 5′-phosphate group with a leaving group. In some forms, the conjugation acid of the leaving group has a pKa of less than 8, such as less than about any of 7.5, 7, 6.5, 6, 5.5, 5, 4.5, 4, 3.5, or 3. In some forms, the leaving group is any one of Cl, or Br, or I, or —OR, or —OC(O)R, or —OS(O)₂R, or —NR¹R², wherein each R is independently haloalkyl, phenyl substituted with one or more alkyl or haloalkyl, or heteroaryl substituted with one or more alkyl or haloalkyl, and wherein R¹ is independently H, alkyl, or haloalkyl, R² is independently haloalkyl, phenyl substituted with one or more alkyl or haloalkyl, or heteroaryl substituted with one or more alkyl or haloalkyl, or R¹ and R² are taken together the N atom to which they are attached to form a heteroaryl. In some forms, the leaving group is

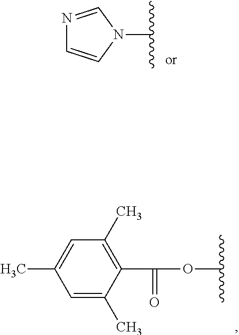 [SMILES: *n1ccnc1 ; *OC(=O)c1c(C)cc(C)cc1C]

wherein the wavy line “ [symbol] ” denotes the attachment of the leaving group to 5′ phosphate of the RNA.

In some forms, an RNA molecule lacking a 5′ phosphate is treated to form a 5′-phosphate group (e.g., on a fragmented 5′ end of the ribonucleic acid). In some forms as provided herein, the methods of the present disclosure utilize enzymatic reactions, driven by polynucleotide kinase, to convert RNA fragments lacking 5′-phosphate group into a RNA comprising a 5′-phosphate group. In some forms, the RNA including a 5′-phosphate group is generated from a fragmented RNA having 5′-OH fragmentation at 5′-terminal end.

In some forms, modification of the RNA includes reacting the 5′-phosphate group with carbonyldiimidazole (CDI) and thereby forming a 5′ phosphate modified with

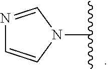 [SMILES: *n1ccnc1]

In some forms, the modification comprises reacting the 5′-phosphate group with 2,4,6-trimethylbenzoyl chloride and thereby forming a 5′ phosphate modified with

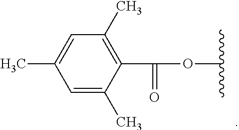 [SMILES: *OC(=O)c1c(C)cc(C)cc1C]

In some forms, the method includes incubating the biological sample including the RNA in 100 mM Carbonyldiimidizol in anhydrous Dimethyl formamide. In some forms, the method includes incubating the biological sample including the RNA in 100 mM Carbonyldiimidizol in anhydrous Dimethyl formamide for about 2 hours at a temperature between 18° C. and 22° C., optionally wherein the temperature is about 20° C. In some forms, after the incubation with Carbonyldiimidizol in anhydrous Dimethyl formamide, the method includes a 10 minute 20° C. incubation in Pyridine. In some forms, after the Pyridine incubation, the method includes incubating the biological sample with 2-aminoethyl methacrylamide (2-AEM) in H₂O to generate modified hydrogel reactive RNA molecules in the biological sample.

In some forms, after the incubation with Carbonyldiimidizol in anhydrous Dimethyl formamide, the method includes washing the biological sample in MeOH (e.g., performing 3 washes in MeOH). In some forms, after the MeOH washes, the method includes incubating the biological sample in 2-AEM, MgCl₂, Triethylamine, Aniline and RNAse inhibitor. In some forms, after the MeOH washes, the method includes a 30-minute 50° C. incubation in 100 mM 2-AEM, 30 mM MgCl₂, 100 mM Triethylamine, 1 nM Aniline and RNAse inhibitor.

In some forms, the method includes contacting the biological sample with a matrix-forming agent. In some forms, the method includes forming a three-dimensional polymerized matrix from the matrix-forming agent, thereby embedding the biological sample. In some forms, the method includes immobilizing the RNA in the three-dimensional polymerized matrix. In some forms, the method includes incubating the biological sample with a polynucleotide kinase (e.g., T4 PNK) and a nucleotide triphosphate prior to incubating the biological sample in the Carbonyldiimidizol in anhydrous Dimethyl formamide.

In some forms of the methods, the method further includes contacting or treating the biological sample and/or target nucleic acid (e.g., RNA) with RNase inhibitors to prevent any undesired fragmentation. In some forms, the biological sample and fragmented ribonucleic acid are treated with a ribonuclease inhibitor. In some forms, the biological sample is contacted with a degradation agent to induce fragmentation, optionally wherein the method further includes treating the biological sample and fragmented ribonucleic acid with a ribonuclease inhibitor after being contacted with the degradation agent. In some forms, the biological sample and fragmented ribonucleic acid are treated with a ribonuclease inhibitor after the biological sample has been contacted with a degradation agent. In some forms, the biological sample is treated with one or more RNase inhibitors. In some forms, the one or more RNase inhibitors are the same. In some forms, the one or more RNase inhibitors are different. Examples of ribonuclease inhibitors include but are not limited to an anti-RNase antibodies, recombinant enzymes, or non-enzymatic inhibitors.

As detailed above, the methods of the present disclosure employ enzymatic conversion of an RNA lacking 5′-phosphate (e.g., a fragmented RNA) into an RNA including a 5′-phosphate group. In some forms, 5′ phosphate group is subsequently modified to enable downstream chemistries and immobilization in the biological sample. In some forms, the RNA lacking a 5′-phosphate is an endogenous RNA that is fragmented in the biological sample, resulting in an RNA including a 5′—OH group.

In some forms, the methods of the present disclosure include contacting the biological sample with a polynucleotide kinase for performing a 5′-end healing reaction (also known as polishing) to phosphorylate a 5′-OH of an RNA in the biological sample. Polynucleotide kinase (PNK) enzymes are present in diverse bacterial taxa. An example listing of PNKs is provided in the SIB Swiss Institute of Bioinformatics Expasy enzyme nomenclature database (entry: EC 2.7.1.78). In some forms, the PNK includes a T4 polynucleotide kinase. In some forms, the PNK is T4 polynucleotide kinase.

In some forms, the PNK is contacted with the biological sample in the presence of magnesium. In some forms, the PNK includes the N-terminal Pnk domain of T4 PNK. In some forms, the PNK is a T4 PNK or a homolog thereof. In some forms, the PNK is a T7 PNK or a homolog thereof. In some forms, the PNK is a Runella slithyformis HD-Pnk or a homolog thereof. In some forms, a biological sample including fragmented ribonucleic acids including a 5′-OH fragmentation at 5′-terminal is treated with a 5′ kinase, such as T4 polynucleotide kinase, thereby resulting in the formation of a 5′-phosphate moiety at 5′-terminal ribose ring.

The methods of the present disclosure encompass the preparation of a ribonucleic acid (RNA) including a 5′-phosphate group or a 5′-phosphate group modified with a leaving group for immobilization of the ribonucleic acids in a matrix. The methods can achieve immobilization of the ribonucleic acids through the use of an attachment agent that mediates the interaction between the ribonucleic acid and the matrix forming agent and ultimately the matrix. In some forms, the methods include contacting the biological sample with one or more attachment agents. In some forms wherein the method includes contacting the biological sample with two or more attachment agents, the attachment agents may be the same or different. In other forms, the attachment agent is a multifunctional molecule. In some forms, the attachment agent includes at least one (e.g., 1, 2, 3, or 4) reactive moiety capable of covalently bonding to the ribonucleic acid and at least one (e.g., 1, 2, 3, or 4) attachment moiety capable of covalently or non-covalently bonding to a matrix-forming agent. It should be recognized that reference to the attachment agent, whether bi- or multifunctional, as defined herein refers to the attachment agent prior to binding with the ribonucleic acid and the matrix-forming agent, unless otherwise noted.

In some forms, the attachment agent is a compound of Formula (I):

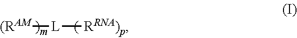

or a salt thereof, wherein:
each R^{RNA} is, independently, a reactive moiety capable of reacting with at least one of the 5′-phosphate group of the RNA, or 5′-phosphate group of the RNA modified with a leaving group; each R^{AM} is, independently, an attachment moiety capable of attaching covalently or noncovalently to a matrix-forming agent; L is a bond or a linker moiety; m is an integer of from 1 to 4, inclusive; and p is an integer of from 1 to 4, inclusive.

In some forms, RRNA, RAM, L, m, and p are each as defined herein. It should be understood that every description, variation, form or aspect of a moiety may be combined with every description, variation, form or aspect of other moieties the same as if each and every combination of descriptions is specifically and individually listed. For example, every description, variation, form or aspect provided herein with respect to RRNA of Formula (I) may be combined with every description, variation, form or aspect of L of Formula (I) the same as if each and every combination were specifically and individually listed. For another example, every description, variation, form or aspect provided herein with respect to RAM of Formula (I) may be combined with every description, variation, form or aspect of L of Formula (I) the same as if each and every combination were specifically and individually listed.

In some forms, at least one reactive moiety is capable of reacting with at least one 5′-phosphate group of the RNA via an enzymatic reaction. In some forms, at least one reactive moiety of the attachment agent includes or is a nucleic acid oligonucleotide including between 2 to 30 (e.g., between any of 2 to 25, 2 to 20, 2 to 15, or 5 to 15) nucleotide residues. In some forms, the nucleic acid oligonucleotide is a DNA oligonucleotide. The nucleic acid oligonucleotide can include any nucleic acid sequence, (e.g., any sequence of nucleotide residues). In some forms, the nucleic acid oligonucleotide includes a random sequence. In some forms, the nucleic acid oligonucleotide includes at least one thymine (T). In some forms, the nucleic acid oligonucleotide includes a sequence of at least 2, 3, 4, 5, 6, 7, 8, or more thymines. In some forms, the nucleic acid oligonucleotide does not include a sequence of thymines. In some forms, p is 4. In some forms, p is 3. In some forms, p is 2. In some forms, p is 1.

In some forms, the attachment moiety RAM is an acrydite. In some forms, the reactive moiety RRNA is a nucleic acid oligonucleotide, such as a DNA. In some forms, the DNA is a sequence of thymine residues. However, the nucleic acid (e.g., DNA oligonucleotide) can include any sequence. In some forms, the nucleic acid oligonucleotide does not include a sequence of thymines. In some forms, the nucleic acid oligonucleotide comprises or is a random sequence. In some forms, the reactive moiety RRNA (e.g., a nucleic acid including a 3′ hydroxyl group) reacts with an RNA including a 5′-phosphate under the catalysis of an RNA ligase, such as T4 RNA Ligase 1.

In some forms, at least one reactive moiety is capable of reacting with at least one 5′-phosphate group or 5′-phosphate group modified with a leaving group of the RNA via a non-enzymatic reaction. In some forms, at least one reactive moiety is capable of reacting with a 5′-phosphate group modified with a leaving group of the RNA via a non-enzymatic reaction, such a substitution reaction.

In some forms, at least one reactive moiety includes or is a nucleophilic group capable of reacting with 5′-phosphate group modified with a leaving group of the ribonucleic acid. In some forms, at least one reactive moiety includes or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some forms, at least one reactive moiety is or includes an amine moiety, an amide moiety, an alcohol moiety, a thiol moiety, a cyano moiety, or an ylide moiety.

In some forms, at least one reactive moiety is or includes an amine moiety (e.g., —NHR or —NH₂), an alcohol moiety, or a thiol moiety that is capable of reacting with a 5′-phosphate group modified with a leaving group. In some forms, the reactive moiety of the attachment agent is or includes an amine moiety (e.g., —NHR or —NH₂). In some forms, the reaction of an amine moiety with a 5′-phosphate group modified with a leaving group of the ribonucleic acid forms a P—NH or —P—NR bond. In some forms, the reactive moiety of the attachment agent is or includes an alcohol moiety (e.g., —OH). In some forms, the reaction of an alcohol moiety with a 5′-phosphate group modified with a leaving group of the ribonucleic acid forms a P—O bond. In some forms, the reactive moiety of the attachment agent is or includes a thiol moiety (e.g., —SH). In some forms, the reaction of a thiol moiety with the 5′-phosphate group modified with a leaving group of the ribonucleic acid forms a P—S bond.

In some forms, the method includes anchoring a 5′ end, a 3′ end, or both a 5′ end and a 3′ end of an RNA to the biological sample or to a matrix embedding the biological sample. In some instances, the method includes anchoring a 5′ end of an RNA to the biological sample or to a matrix embedding the biological sample according to any of the forms described herein, and further includes anchoring 3′ end of the RNA to the matrix. In some forms, anchoring 3′ end of the RNA to the matrix includes contacting the biological sample with a probe that hybridizes to the 3′ end of the RNA (e.g., a probe including a plurality of thymine bases that hybridizes to a poly(A) tail of the RNA). In some forms, anchoring 3′ end of the RNA includes contacting the biological sample including the RNA with a formylation reagent, wherein the RNA includes a 2′,3′-vicinal diol and the formylation reagent converts the 2′,3′-vicinal diol moiety into a 2′3′-dialdehyde moiety; and contacting the biological sample with a 3′-end attachment agent including at least one aldehyde-reactive group capable of reacting with at least one aldehyde of the 2′,3′-dialdehyde moiety of the RNA to form a covalent bond and an attachment moiety capable of attaching covalently or non-covalently to an exogenous or endogenous molecule in the biological sample (e.g., to a matrix such as a gel).

In some forms, 3′-end attachment agent includes an aldehyde-reactive group. The aldehyde reactive group typically reacts with at least one aldehyde of the 2′,3′-dialdehyde moiety of the RNA to form a covalent bond, and the attachment moiety typically attaches covalently or non-covalently to an exogenous or endogenous molecule. In some forms, the aldehyde-reactive group includes or is a nucleophilic group capable of reacting with at least one aldehyde of the 2′,3′-dialdehyde moiety of the ribonucleic acid. In some forms, the reactive group includes or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some forms, the aldehyde-reactive group or first reactive group of the attachment agent is or includes an amine moiety, an amide moiety, an alcohol moiety, a thiol moiety, a cyano moiety, or an ylide moiety. In some forms, the aldehyde-reactive group of 3′-end attachment agent is or includes an amine moiety (e.g., —NHR or —NR2). In some forms, the reaction of an amine moiety with an aldehyde moiety of the ribonucleic acid forms an imine or an enamine. In some forms, the method includes reduction of the imine (e.g., with NaBH4, optionally with 0.2M NaBH4).

In some forms of Formula (I), L is a bond. In some forms of Formula (I), L is a linker moiety. In some form, L includes or is an unbranched or branched C1-C150 alkylene, which can be interrupted by 1 to 50 independently selected O, NH, N, S, C6-C12 arylene, or 5- to 12-membered heteroarylene. In some forms, L includes or is an unbranched and uninterrupted C1-C150 alkylene. In some forms, L includes or is a branched and uninterrupted C1-C150 alkylene. In some forms, L includes or is an unbranched C1-C150 alkylene interrupted by 1 to 50 NH, O, or S.

In some forms, the methods include:
(a) contacting the biological sample including the target nucleic acid (e.g., ribonucleic acid) with T4 polynucleotide kinase, wherein the T4 polynucleotide kinase catalyzes formation of a 2′,3′-vicinal diol moiety on the fragmented ribonucleic acid;
(b) contacting the biological sample with sodium (meta) periodate, and wherein the sodium (meta) periodate converts the 2′,3′-vicinal diol to a 2′3′-dialdehyde moiety;
(c) contacting the biological sample with N-(2-aminoethyl) methacrylamide, 2-aminoethyl methacrylate, or 2-aminoethyl (E)-but-2-enoate and sodium borohydride, wherein the N-(2-aminoethyl) methacrylamide, 2-aminoethyl methacrylate, or 2-aminoethyl (E)-but-2-enoate reacts with at least one aldehyde of the 2′3′-dialdehyde moiety of the ribonucleic acid to form 3′-aminoethylene-methacrylamide, 3′-aminoethylene-methacrylate, or 3′-aminoethyl (E)-but-2-enoate;
(d) contacting the biological sample with a matrix-forming agent; and
(e) forming a polymerized matrix from the matrix-forming agent, thereby embedding the biological sample in the three-dimensional polymerized matrix and anchoring the ribonucleic acid to the three-dimensional polymerized matrix.

In some forms, the 3′-aminoethylene-methacrylamide, 3′-aminoethylene-methacrylate, or 3′-aminoethyl (E)-but-2-enoate typically reacts with the matrix-forming agent to form a covalent bond.

In some forms, the T4 polynucleotide kinase polishes 3′ RNA ends into vicinal diols, NaIO₄ oxidizes the diols into aldehydes, 2-AEM uses the amino group to react with the aldehydes, adding 2 methacrylamide groups to all RNA fragments (including e.g., mRNA, lncRNA, miRNA). In some cases, aniline can be added as a catalyst and may help imine formation. In some cases, the resulting imine linkage to the RNA can be readily hydrolyzed. In some forms, the sample is treated including reduction with NaBH₄ to make the processing irreversible. In some forms, the NaBH₄ is suspended in water and treated with the sample. In some forms, the NaBH₄ is suspended in ethanol and treated with the sample.

In some forms, the 2′,3′-vicinal diol is generated from a fragmented RNA having a 2′,3′cyclo-phosphate fragmentation at 3′-terminal end or a 2′ hydroxyl and a 3′ phosphate fragmentation at 3′-terminal end. In some forms, the 2′,3′-vicinal diol is generated from a fragmented RNA having a 2′,3′cyclo-phosphate fragmentation at 3′-terminal end. In some forms, the 2′,3′-vicinal diol is generated from a fragmented RNA having a 2′ hydroxyl and a 3′ phosphate fragmentation at 3′-terminal end. In some forms, the 2′,3′-vicinal diol is provided by contacting a fragmented RNA with a 3′ phosphatase. In some forms, the methods include contacting a fragmented RNA, wherein the fragmented RNA includes a 2′,3′cyclo-phosphate fragmentation at 3′-terminal end or a 2′ hydroxyl and a 3′ phosphate fragmentation at 3′-terminal end, with a 3′-phosphatase to generate a fragmented RNA including a 2′,3′-vicinal diol.

Examples of matrix-forming agents include acrylamide, bisacrylamide, cellulose, alginate, polyamide, agarose, dextran, or polyethylene glycol. The matrix-forming agents can form a matrix by three-dimensional polymerization and/or crosslinking of the matrix-forming agents using methods specific for the matrix-forming agents and methods, reagents and conditions. In some forms, the three-dimensional polymerized matrix is formed by subjecting the matrix-forming agent to polymerization (or to further polymerization, in the case of matrix-forming agents that are polymers such as polyethylene glycol). In some forms, the matrix includes polyacrylamide, cellulose, alginate, polyamide, cross-linked agarose, cross-linked dextran, cross-linked polyethylene glycol, or a combination thereof. Forming a three-dimensional polymerized matrix can include adding a polymerization inducing catalyst, UV or functional cross-linker to allow the formation of the three-dimensional polymerized matrix.

### D. Labelling, Staining and Imaging

Any of the described methods can include one or more steps of imaging and/or staining the biological sample at one or more stages. In some forms, the methods label the biological sample prior to the initiation of the described methods for enhanced spatial profiling of interactions between nucleic acids and nucleic acid-binding proteins in the sample. For example, in some forms, subsequent to step (a) of the described methods, one or more steps of imaging and/or de-staining a labelled sample are carried out.

The described methods can include any one or more of these steps at any applicable point during the methods. For example, in some forms, the methods include one or more steps of (1) imaging and/or staining the biological sample; and/or (2) preparation of samples for the application of probes and/or oligonucleotides, e.g., by de-paraffinization or dewaxing by permeabilization. (1) and/or (2) is typically before or after any one or more of method steps (a), (b), (c), (d), (e), (f), (g) or (h).

In some forms, the described methods for spatially detecting an interaction between a nucleic acid and a non-nucleic acid analyte in a biological sample (e.g., present in a biological sample), include: prior to step (a) staining and/or imaging a biological sample; and/or permeabilizing (e.g., providing a solution including a permeabilization reagent to) the biological sample; the (a) contacting the biological sample with a plurality of first probes according to the described methods.

Biological samples can be stained using a wide variety of stains and staining techniques. In some forms, the biological sample is a section on a slide (e.g., a 10, 12 or 15 μm section). In some forms, the biological sample is dried after placement onto a glass slide. In some forms, the biological sample is dried at 42° C. In some forms, drying occurs for about 1 hour, about 2, hours, about 3 hours, or until the sections become transparent. In some forms, the biological sample can be dried overnight (e.g., in a desiccator at room temperature).

In some forms, a sample is stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some forms, the methods include imaging the biological sample. In some forms, imaging the sample occurs prior to deaminating the biological sample. In some forms, the sample is stained using staining techniques known in the art, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some forms, the stain is an H&E stain.

In some forms, the biological sample is stained using a detectable label (e.g., radioisotopes, fluorophores, chemiluminescent compounds, bioluminescent compounds, and dyes) as described elsewhere herein. In some forms, a biological sample is stained using only one type of stain or one technique. In some forms, staining includes biological staining techniques such as H&E staining. In some forms, staining includes identifying analytes using fluorescently-conjugated antibodies. In some forms, a biological sample is stained using two or more different types of stains, or two or more different staining techniques. For example, in some forms a biological sample can be prepared by staining and imaging using one technique (e.g., H&E staining and brightfield imaging), followed by staining and imaging using another technique (e.g., IHC/IF staining and fluorescence microscopy) for the same biological sample.

In some forms, biological samples are de-stained after staining. Methods of de-staining or discoloring a biological sample are known in the art, and generally depend on the nature of the stain(s) applied to the sample. For example, H&E staining can be de-stained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof).

In some forms, the de-staining includes 1, 2, 3, 4, 5, or more washes in an acid (e.g., HCl). In some forms, de-staining includes adding HCl to a downstream solution (e.g., permeabilization solution). In some forms, de-staining includes dissolving an enzyme used in the disclosed methods (e.g., pepsin) in an acid (e.g., HCl) solution. In some forms, after de-staining hematoxylin with an acid, other reagents can be added to the de-staining solution to raise the pH for use in other applications. For example, SDS can be added to an acid de-staining solution in order to raise the pH as compared to the acid de-staining solution alone. As another example, in some forms, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Exemplary methods for multiplexed staining and de-staining may be carried out, for example, as described in Bolognesi, et al., J. Histochem. Cytochem. 2017; 65(8):431-444, Lin, et al., Nat Commun. 2015; 6:8390, Pirici, et al., J. Histochem. Cytochem. 2009; 57:567-75, and Glass et al., J. Histochem. Cytochem. 2009; 57:899-905, the entire contents of each of which are incorporated herein by reference.

In some forms, immunofluorescence or immunohistochemistry protocols (direct and indirect staining techniques) are performed as a part of, or in addition to, the exemplary spatial workflows presented herein. For example, in some forms, tissue sections can be fixed according to methods described herein. The biological sample can be transferred to an array (e.g., capture probe array), whereby analytes (e.g., proteins) are probed using immunofluorescence protocols. For example, in some forms, the sample is rehydrated, blocked, and permeabilized (e.g., using 3× saline-sodium citrate (SSC), 2% BSA, 0.1% Triton X, 1 U/μl RNAse inhibitor for 10 minutes at 4° C.) before being stained with fluorescent primary antibodies (e.g., using 1:100 in 3×SSC, 2% BSA, 0.1% Triton X, 1 U/μl RNAse inhibitor for 30 minutes at 4° C.). In some forms, the biological sample is washed, cover-slipped (in glycerol+1 U/μl RNAse inhibitor), imaged (e.g., using a confocal microscope or other apparatus capable of fluorescent detection), washed again, and processed according to analyte capture or spatial workflows described herein. In some forms, a glycerol solution and a cover slip are added to the sample. In some forms, the glycerol solution includes a counterstain (e.g., DAPI). As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some forms, optimizing permeabilization is useful for identifying intracellular analytes. Permeabilization optimization can include selection of permeabilization agents, concentration of permeabilization agents, and permeabilization duration. Tissue permeabilization is discussed elsewhere herein.

### E. Additional Methodologies

Any of the described methods can include one or more additional steps that further enhance or otherwise facilitate the spatial profiling of interactions between nucleic acids and nucleic acid-binding proteins in the sample.

For example, as described herein, in some forms the methods include one or more steps of washing the sample, selectively conjugating an analyte with an analyte binding moiety, hybridizing nucleic acids, permeabilizing the sample, etc. Each of these processes can be carried out as part of one or more of the described method steps. For example, the additional methodologies can be applied during, before or after any one or more of the described method steps (a), (b), (c), (d), (e), (f), (g) or (h). Additional details of these methodologies are provided below.

### 1. Preparation of the Sample for Probes

In some forms, the methods include one or more steps for preparation of the biological sample for application of probes. For example, in some forms, the biological sample is deparaffinized and/or de-crosslinked. In some forms, one or more steps to de-paraffinize a sample is carried out prior to contacting the biological sample with one or more first probes (e.g., prior to step (a)). In some forms, one or more steps to de-cross-link a sample is carried out after embedding the biological sample in a matrix (e.g., following step (c)).

### (a.) De-Paraffinizing a Sample

In some forms, the methods include one or more steps to de-paraffinize a biological sample that includes paraffin wax. Typically, sample paraffinization is achieved using any method known in the art. For example, in some forms, the biological sample is treated with a series of washes that include xylene and various concentrations of ethanol. In some forms, methods of deparaffinization include treatment of xylene (e.g., three washes at 5 minutes each). In some forms, the methods further include treatment with ethanol (e.g., 100% ethanol, two washes 10 minutes each; 95% ethanol, two washes 20 minutes each; 70% ethanol, two washes 10 minutes each; 50% ethanol, two washes 10 minutes each). In some forms, after ethanol washes, the biological sample can be washed with deionized water (e.g., two washes for 5 minutes each). It is appreciated that one skilled in the art can adjust these methods to optimize deparaffinization.

### (b.) De-Crosslinking a Sample

In some forms, the biological sample is de-crosslinked. For example, in some forms, the biological sample is de-crosslinked in a solution containing TE buffer (including Tris and EDTA). In some forms, the TE buffer is basic (e.g., at a pH of about 9). In some forms, de-crosslinking occurs at about 50° C. to about 80° C. In some forms, de-crosslinking occurs at about 70° C. In some forms, de-crosslinking occurs for about 1 hour at 70° C. For example, in some forms, just prior to de-crosslinking, the biological sample is treated with an acid (e.g., 0.1M HCl for about 1 minute). After the decrosslinking step, the biological sample can be washed (e.g., with 1×PBST).

### (c.) Permeabilizing a Sample

In some forms, the biological sample is permeabilized before, during or after one or more of steps (a), (b), (c), (d) or (e). In some forms, the methods of preparing a biological sample for probe application include permeabilizing the sample. In some forms, the biological sample is permeabilized using a phosphate buffer. In some forms, the phosphate buffer is PBS (e.g., 1×PBS). In some forms, the phosphate buffer is PBST (e.g., 1×PBST). In some forms, the permeabilization step is performed multiple times (e.g., 3 times at 5 minutes each).

In some forms, permeabilization occurs using a protease. In some forms, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some forms, the endopeptidase is pepsin. In some forms, the protease is proteinase K.

In some forms, after creating a combined probe (e.g., by ligating a first probe and/or a second probe that are hybridized to adjacent sequences in a splint), the biological sample is permeabilized. In some forms, the biological sample is permeabilized contemporaneously with or prior to contacting the biological sample with a first probe and/or a second probe, e.g., hybridizing the first probe and the second probe to the provide a combined probe, and then permeabilizing the sample to release the combined product from the analytes in sample.

In some forms, methods provided herein include permeabilization of the biological sample such that a probe/bound analyte can more easily bind to the immobilized capture probe (e.g., compared to no permeabilization).

In some forms, the permeabilization step includes application of a permeabilization buffer to the biological sample. In some forms, the permeabilization buffer includes a buffer (e.g., Tris pH 7.5), MgCl₂, sarkosyl detergent (e.g., sodium lauroyl sarcosinate), enzyme (e.g., proteinase K, and nuclease free water. In some forms, the permeabilization step is performed at 37° C. In some forms, the permeabilization step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 40 minutes.

In some forms, after generating a combined probe/ligation product, the combined probe/ligation product is released from the analyte. In some forms, a combined probe/ligation product is released from the analyte using an endoribonuclease. In some forms, the endoribonuclease is RNase H, RNase A, RNase C, or RNase I. In some forms, the endoribonuclease is RNase H. RNase H is an endoribonuclease that specifically hydrolyzes the phosphodiester bonds of RNA, when hybridized to DNA.

RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA: DNA heteroduplex. In some forms, the RNase His RNase H1, RNase H2, or RNase H1, or RNase H2. In some forms, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli. In some forms, the releasing step is performed using a releasing buffer. In some forms, the release buffer includes one or more of a buffer (e.g., Tris pH 7.5), enzyme (e.g., RNAse H) and nuclease-free water.

In some forms, the releasing step is performed at 37° C. In some forms, the releasing step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 30 mins. In some forms, the releasing step occurs before the permeabilization step. In some forms, the releasing step occurs after the permeabilization step. In some forms, the releasing step occurs at the same time as the permeabilization step (e.g., in the same buffer).

### (d.) Blocking a Sample

In some forms, the methods of preparing a biological sample for probe application include steps of equilibrating and blocking the biological sample. In some forms, equilibrating is performed using a pre-hybridization (pre-Hyb) buffer. In some forms, the pre-Hyb buffer is RNase-free. In some forms, the pre-Hyb buffer contains no bovine serum albumin (BSA), solutions like Denhardt's, or other potentially nuclease-contaminated biological materials.

In some forms, the equilibrating step is performed multiple times (e.g., 2 times at 5 minutes each; 3 times at 5 minutes each). In some forms, the biological sample is blocked with a blocking buffer. In some forms, the blocking buffer includes a carrier such as tRNA, for example yeast tRNA such as from brewer's yeast (e.g., at a final concentration of 10-20 μg/mL). In some forms, blocking can be performed for 5, 10, 15, 20, 25, or 30 minutes.

Any of the foregoing steps can be optimized for performance. For example, one can vary the temperature. In some forms, the pre-hybridization methods are performed at room temperature. In some forms, the pre-hybridization methods are performed at 4° C. (in some forms, varying the timeframes provided herein).

In some forms, the capture domain of a capture probe or the capture domain binding site of a second probe is blocked prior to binding and/or adding a first probe or second probe to a biological sample. This prevents the first and/or second probe capture domain binding sequences from prematurely hybridizing, for example, prior to analyte binding.

Therefore, in some forms, a blocking probe is used to block or modify the free 3′ end of the capture domain of a capture probe, or the capture domain binding site of a second probe. In some forms, a blocking probe can be hybridized to the capture domain of the second probe to mask the free 3′ end of the capture domain. In some forms, a blocking probe can be a hairpin probe or partially double stranded probe. In some forms, the free 3′ end of the capture domain, or of the second probe capture domain binding sequence can be blocked by chemical modification, e.g., addition of an azidomethyl group as a chemically reversible capping moiety such that the capture domains do not include a free 3′ end.

Blocking or modifying the capture domain or the capture domain binding site of a second probe, particularly at the free 3′ end of the capture domain, prior to contacting with a first, second or combined probe, prevents undesired or premature hybridization of the second probe capture domain binding sequence to the capture domain (e.g., prevents the capture of a poly(A) of a capture domain to a poly(T) capture domain). In some forms, a blocking probe can be referred to as a capture domain blocking moiety.

In some forms, the blocking probes can be reversibly removed. For example, blocking probes can be applied to block the free 3′ end of either or both the capture domain, or second probe capture domain binding sequence. Blocking interaction between the capture domains can reduce non-specific capture to the capture probes. After the second probe hybridizes to an analyte (e.g., a nucleic acid) (and is optionally docked and/or ligated to a first probe), the blocking probes can be removed from 3′ end of the capture domain and/or the capture probe, and the combined probe/ligation product can migrate to and become bound by a capture probe (e.g., immobilized on a substrate). In some forms, the removal includes denaturing the blocking probe from capture domain and/or first oligonucleotide or second oligonucleotide capture domain binding sequence. In some forms, the removal includes removing a chemically reversible capping moiety. In some forms, the removal includes digesting the blocking probe with an RNase (e.g., RNase H).

In some forms, the blocking probes are oligo (dT) blocking probes. In some forms, the oligo (dT) blocking probes can have a length of 15-30 nucleotides. In some forms, the oligo (dT) blocking probes can have a length of 10-50 nucleotides, e.g., 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 nucleotides. In some forms, the capture domain or capture domain binding site can be blocked at different temperatures (e.g., 4° C. and 37° C.).

### (e.) Washing a Sample

In some forms, the methods disclosed herein also include a wash step. In some forms, a wash step removes any unbound oligonucleotides, and/or any unbound protein-binding moieties. Wash steps could be performed between any of the steps in the methods disclosed herein. For example, a wash step can be performed after adding probes or oligonucleotides to the biological sample. As such, free/unbound probes or oligonucleotides are washed away, leaving only probes or oligonucleotides that have hybridized to an analyte. In some forms, multiple (e.g., at least 2, 3, 4, 5, or more) wash steps occur between the methods disclosed herein. Wash steps can be performed at times (e.g., 1, 2, 3, 4, or 5 minutes) and temperatures (e.g., room temperature; 4° C.) known in the art and determined by a person of skill in the art. In some forms, wash steps are performed using a wash buffer. In some forms, the wash buffer includes SSC (e.g., 1×SSC). In some forms, the wash buffer includes PBS (e.g., 1×PBS). In some forms, the wash buffer includes PBST (e.g., 1×PBST). In some forms, the wash buffer can also include formamide or be formamide free.

### (f.) Processing for Sequence Determination

In some forms, the step of determining a sequence for two or more components of a captured, combined probe (e.g., as in step (h)) includes amplifying all or part of the combine probe/ligation product specifically bound to the capture domain of a capture probe.

Therefore, in some forms, a step of determining includes amplifying a combined probe to produce an amplified product. An exemplary amplified product includes (i) all or part of sequence of the combine probe/ligation product specifically bound to the capture domain, or a complement thereof, and (ii) all or a part of the sequence of the spatial barcode, or a complement thereof.

In some forms, the determining step includes sequencing. In some forms, the sequencing step includes in situ sequencing, Sanger sequencing methods, next-generation sequencing methods, and/or nanopore sequencing.

After a first probe and second probe bound to an analyte, such as a nucleic acid or complement thereof, or combine probe/ligation product has hybridized or otherwise been captured on a capture probe (e.g., an immobilized capture probe) according to any of the methods described above in connection with the general spatial analytical methodology, the barcoded constructs that result from the recapture step are analyzed.

In some forms, the methods further include subjecting a region of interest in the biological sample to spatial transcriptomic analysis. In some forms, one or more of the capture probes includes a unique molecular identifier (UMI). In some forms, one or more of the capture probes includes a cleavage domain. In some forms, the cleavage domain includes a sequence recognized and cleaved by a uracil-DNA glycosylase, apurinic/apyrimidinic (AP) endonuclease (APE1), U uracil-specific excision reagent (USER), and/or an endonuclease VIII. In some forms, one or more capture probes do not include a cleavage domain and therefore, for example, is not cleaved from an array.

In some forms, a capture probe bound to a combined probe (“captured probe”) can be extended (an “extended captured probe,” e.g., as described herein). Therefore, in some forms, the methods include extending a capture probe bound to a combined probe to provide an extended captured probe. For example, extending a capture probe bound to a combined probe can include generating cDNA from a captured (hybridized) nucleic acid hybridized to the complementary region of the second probe. An exemplary captured nucleic acid is an RNA, such as an mRNA. This process typically involves synthesis of a complementary strand of the hybridized nucleic acid, e.g., generating cDNA based on the captured RNA template (the RNA hybridized to the capture domain of the capture probe). Thus, in an initial step of extending a captured probe, e.g., the cDNA generation, the captured (hybridized) nucleic acid, e.g., RNA, acts as a template for the extension, e.g., reverse transcription, step.

In some forms, reverse transcription (RT) reagents can be added to permeabilized biological samples. Incubation with the RT reagents can produce spatially-barcoded full- or partial-length cDNA from the captured analytes (e.g., polyadenylated mRNA). Second strand reagents (e.g., second strand primers, enzymes) can be added to the biological sample on the slide to initiate second strand synthesis.

In some forms, the second probe, or the combine probe/ligation product thereof, and/or the capture probe bound thereto is extended using reverse transcription. For example, reverse transcription includes synthesizing cDNA (complementary or copy DNA) from RNA, e.g., (messenger RNA), using a reverse transcriptase. In some forms, reverse transcription is performed while the tissue is still in place, generating an analyte library, where the analyte library includes the spatial barcodes from the associated capture probes. In some forms, a captured probe is extended using one or more DNA polymerases. In some forms, an analyte capture domain of a first probe, or a second probe, and/or a capture domain of a capture probe includes a primer for producing the complementary strand of a nucleic acid hybridized to the probe, e.g., a primer for DNA polymerase and/or reverse transcription. The nucleic acid, e.g., DNA and/or cDNA, molecules generated by the extension reaction incorporate the sequence of the first probe, second probe or capture probe, respectively. The extension of the captured probe, e.g., a DNA polymerase and/or reverse transcription reaction, can be performed using a variety of suitable enzymes and protocols.

In some forms, a full-length DNA (e.g., cDNA) molecule is generated. In some forms, a “full-length” DNA molecule refers to the whole of a “captured” nucleic acid molecule. However, if a nucleic acid (e.g., RNA) was partially degraded in the tissue sample, then the captured nucleic acid molecules will not be the same length as the initial RNA in the tissue sample. In some forms, 3′ end of an extended captured probe, e.g., first strand cDNA molecules, is modified. For example, a linker or adaptor can be ligated to the 3′ end of the extended captured probes. This can be achieved using single stranded ligation enzymes such as T4 RNA ligase or Circligase™ (available from Lucigen, Middleton, WI).

In some forms, template switching oligonucleotides are used to extend cDNA in order to generate a full-length cDNA (or as close to a full-length cDNA as possible). In some forms, a second strand synthesis helper probe (a partially double stranded DNA molecule capable of hybridizing to the 3′ end of the extended captured probe), can be ligated to the 3′ end of the extended captured probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9°N) DNA ligase (9° N™ DNA ligase, New England Biolabs), Ampligase™ (available from Lucigen, Middleton, WI), and SplintR (available from New England Biolabs, Ipswich, MA). In some forms, a polynucleotide tail, e.g., a poly(A) tail, is incorporated at the 3′ end of the extended captured probe molecules. In some forms, the polynucleotide tail is incorporated using a terminal transferase active enzyme.

### (i.) Sequence Amplification

In some forms, double-stranded extended captured probes are treated to remove any un-extended captured probes prior to amplification and/or analysis, e.g., sequence analysis. This can be achieved by a variety of methods, e.g., using an enzyme to degrade the un-extended captured probes, such as an exonuclease enzyme, or purification columns.

In some forms, extended captured probes are amplified to yield quantities that are sufficient for analysis, e.g., via DNA sequencing. In some forms, the first strand of the extended captured probes (e.g., DNA and/or cDNA molecules) acts as a template for the amplification reaction (e.g., a polymerase chain reaction).

In some forms, the amplification reaction incorporates an affinity group onto the extended capture probe (e.g., RNA-cDNA hybrid) using a primer including the affinity group. In some forms, the primer includes an affinity group and the extended captured probes includes the affinity group. The affinity group can correspond to any of the affinity groups described previously.

In some forms, amplifying the extended captured probes can function, e.g., to release the extended captured probes from the surface of a substrate, insofar as copies of the extended probes are not immobilized on the substrate.

In some forms, the extended capture probe or complement or amplicon thereof is released. The step of releasing the extended captured probe or complement or amplicon thereof from the surface of the substrate can be achieved in a number of ways. In some forms, an extended captured probe or a complement thereof is released from the array by nucleic acid cleavage and/or by denaturation (e.g., by heating to denature a double stranded molecule).

In some forms, the extended captured probe or complement or amplicon thereof is released from a surface, e.g., of a substrate (e.g., array) by physical means. For example, where the extended captured probe is indirectly immobilized on an array substrate, e.g., via hybridization to a surface probe, it can be sufficient to disrupt the interaction between the extended captured probe and the surface probe. Methods for disrupting the interaction between nucleic acid molecules include denaturing double stranded nucleic acid molecules are known in the art. One method for releasing the DNA molecules (i.e., of stripping the array of extended probes) is to use a solution that interferes with the hydrogen bonds of the double stranded molecules. In some forms, the extended captured probe is released by an applying heated solution, such as water or buffer, of at least 85° C., e.g., at least 90, 91, 92, 93, 94, 95, 96, 97, 98, or 99° C. In some forms, a solution including salts, surfactants, etc. that can further destabilize the interaction between the nucleic acid molecules is added to release the extended captured probe from a substrate.

In some forms, where the extended captured probe includes a cleavage domain, the extended capture probe is released from a surface of a substrate by cleavage. For example, the cleavage domain of the extended captured probe can be cleaved by any of the methods described herein. In some forms, the extended captured probe is released from the surface of the substrate, e.g., via cleavage of a cleavage domain in the extended captured probe, prior to the step of amplifying the extended captured probe.

### (ii.) Template Switching Oligonucleotides

In some forms, the captured analytes can be spatially-barcoded by performing a reverse transcriptase first strand cDNA reaction using template switching oligonucleotides. For example, a template switching oligonucleotide (TSO) can hybridize to a poly(C) tail added to a 3′end of the cDNA by a reverse transcriptase enzyme in a template-independent manner. The hybridized TSO is used to further extend the first strand cDNA such that it includes a complement of the TSO. The original nucleic acid template and template switching oligonucleotide can then be denatured from the extended cDNA and the spatially-barcoded capture probe can then hybridize with the cDNA and a complement of the cDNA can be generated. In some forms, the TSO (or a primer having a similar sequence thereto) can be used to prime synthesis of a second strand cDNA templated from the first strand cDNA. The first strand cDNA can then be purified and collected for downstream amplification steps. The first strand cDNA can be amplified using PCR, where the forward and reverse primers flank the spatial barcode and analyte regions of interest, generating a library associated with a particular spatial barcode. In some forms, the library preparation can be quantitated and/or quality controlled to verify the success of the library preparation steps.

A “template switching oligonucleotide” is an oligonucleotide that hybridizes to untemplated nucleotides added by a reverse transcriptase (e.g., enzyme with terminal transferase activity) during reverse transcription. In some forms, a template switching oligonucleotide hybridizes to untemplated poly(C) nucleotides added by a reverse transcriptase. In some forms, the template switching oligonucleotide adds a common 5′ sequence to full-length cDNA that is used for cDNA amplification.

In some forms, the template switching oligonucleotide adds a common sequence onto the 5′ end of an RNA being reverse transcribed. For example, a template switching oligonucleotide can hybridize to untemplated poly(C) nucleotides added onto the end of a cDNA molecule and provide a template for the reverse transcriptase to continue replication to 5′ end of the template switching oligonucleotide, thereby generating full length cDNA ready for further amplification.

In some forms, once a full-length cDNA molecule is generated, the template switching oligonucleotide can serve as a primer in a cDNA amplification reaction. In some forms, a template switching oligonucleotide is added before, contemporaneously with, or after a reverse transcription, or other terminal transferase-based reaction. In some forms, a template switching oligonucleotide is included in the capture probe. In certain forms, methods of sample analysis using template switching oligonucleotides can involve the generation of nucleic acid products from analytes of the tissue sample, followed by further processing of the nucleic acid products with the template switching oligonucleotide.

Template switching oligonucleotides can include a hybridization region and a template region. The hybridization region can include any sequence capable of hybridizing to the target. In some forms, the hybridization region can, e.g., include a series of G bases to complement the overhanging C bases at the 3′ end of a cDNA molecule. The series of G bases can include 1 G base, 2 G bases, 3 G bases, 4 G bases, 5 G bases, or more than 5 G bases. The template sequence can include any sequence to be incorporated into the cDNA.

In other forms, the hybridization region can include at least one base in addition to at least one G base. In other forms, the hybridization can include bases that are not a G base. In some forms, the template region includes at least 1 (e.g., at least 2, 3, 4, 5 or 10 more) tag sequences and/or functional sequences. In some forms, the template region and hybridization region are separated by a spacer.

In some forms, the template regions include a barcode sequence. The barcode sequence can act as a spatial barcode and/or as a unique molecular identifier. Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ fluoro bases (e.g., Fluoro C, Fluoro U, Fluoro A, and Fluoro G), or any combination of the foregoing.

In some forms, the length of a template switching oligonucleotide can be at least about 1, 2, 10, 20, 50, 75, 100, 150, 200, or 250 nucleotides or longer. In some forms, the length of a template switching oligonucleotide can be at most about 2, 10, 20, 50, 100, 150, 200, or 250 nucleotides or longer.

### III. Compositions for Profiling of Analyte-Analyte Interactions

Compositions for use in the disclosed methods for enhanced spatial profiling of interactions between nucleic acids and nucleic acid-binding proteins are provided. Typically, the compositions include a first probe; a second probe (and optionally a splint oligonucleotide); and a capture probe.

The compositions include a first probe configured to include at least the following components:
(i) an analyte capture region (e.g., a target protein-binding moiety), including a moiety configured to selectively capture a nucleic acid-binding analyte directly or indirectly, such as a nucleic acid-binding protein, or a protein that interacts with a nucleic acid-binding protein; and
(ii) an oligonucleotide, including:
   (a) a docking motif for selective interaction (e.g., hybridization) with a second probe;
   (b) optionally a barcode or other motif specific for identification of the analyte capture region or the targeted analyte.

The compositions include a second probe configured to include at least the following components:
(iii) a docking motif for selective interaction (e.g., hybridization) with a first probe; and
(iv) a region complementary to one or more nucleic acids in a sample; and
(v) a capture domain binding sequence, e.g., complementary to the capture domain of a capture probe.

In some forms, the compositions include a splint oligonucleotide configured to include at least the following components:
(vi) a docking motif for selective interaction with a first probe; and
(vii) a docking motif for selective interaction with a second probe.

The compositions include an array including a plurality of capture probes wherein a capture probe in the plurality of capture probes includes at least the following components:
(viii) a capture domain (e.g., configured to hybridize to the capture domain binding sequence of a second probe); and
(ix) a spatial barcode.

The various features needed for the compositions used in the disclosed methods can be implemented in one, two, or three distinct probes, together with capture probes associated with a substrate.

In some forms, the methods utilize all the components within a single probe, together with an array of capture probes. In other forms, the methods components are present collectively within a first and a second probe, together with an array of capture probes. In further forms, the methods utilize a first and a second probe and a splint oligonucleotide that collectively includes all the elements. Any of the oligonucleotides can further include one or more additional elements such sequencing or PCR primer binding sites.

FIGS. 12A-12D are schematic diagrams depicting each of four different exemplary first probe structures (131010), respectively, each configured to selectively bind to the same nucleic acid-binding analyte (13200) that is in contact with/conjugated to the same nucleic acid (131040). The target nucleic acid is depicted as being fragmented, i.e., having two physically distinct fragments (13191) and (13190), one of which (13191) is depicted as being bound to the nucleic acid-binding analyte (13200).

Each of the first probes (131010) are depicted including two functionally distinct domains, i.e., a nucleic acid-binding moiety capture agent (132001 or 13116) and a single-stranded oligonucleotide (131011). In some forms, as depicted in FIG. 12A, the nucleic acid-binding moiety capture agent (132001) is typically coupled to the 5′ terminus of a single-stranded oligonucleotide (131011) component via a linker (13100). The single-stranded oligonucleotide (131011) includes a second probe docking sequence (13130), and optionally a target agent identification sequence (e.g., a barcode) (13110), and one or more additional functional domains (131002 and 13120). The capture agent can be a nucleic acid, such as an aptamer (see (132001) depicted in FIGS. 12A-12B, 12E), or a protein such as an antibody or antigen binding fragment thereof (see (13116) depicted in FIGS. 12C-12D).

The first probe also includes one or more sites that are functionalized, for example, by conjugation with a moiety (★) for embedding the probe within a matrix, such as a gel. In some forms, as depicted in FIG. 12A, the moiety is conjugated in the region of the nucleic acid-binding moiety capture agent and can either be conjugated directly to 5′ end of the capture agent (132001), or it can be conjugated at the 5′ end of an oligonucleotide associated with the capture agent (132002), e.g., which itself is conjugated to the capture agent via a linker (13115).

In other forms, as depicted in FIG. 12C, the moiety is conjugated in the region of the nucleic acid-binding moiety capture agent via conjugation to the 3′ terminus of a first oligonucleotide tag (132004) that is hybridized to the 5′ region of the single-stranded oligonucleotide, for example, by complementary paring with the nucleotides in a functional domain (131002).

In other forms, as depicted in FIG. 12D, the moiety is conjugated in the region of the nucleic acid-binding moiety capture agent that is hybridized to complementary bases in to the 5′ terminus of a second oligonucleotide tag (132122) that is hybridized to complementary bases in the 3′ terminus region (132005) of a first oligonucleotide tag (132004) that is itself hybridized to single-stranded oligonucleotide, for example, by complementary paring with the nucleotides in a functional domain (131002).

In some forms, as depicted in FIG. 12B, the capture agent is also optionally associated with the single-stranded oligonucleotide via an oligonucleotide tag (132003) that is hybridized to the single-stranded oligonucleotide, for example, by complementary paring with the nucleotides in a functional domain (131002). In some forms, the capture domain is associated with the oligonucleotide tag via a linker (13100).

In further forms, as depicted in FIG. 12E, a single bi-specific probe includes the necessary components of the first probe of any of FIGS. 12A-12D, as well as a nucleic acid-binding domain (13170) and a capture domain binding sequence (13180), as well as optionally a bridging or spanning domain (13139).

Schematic diagrams depicting the binding of a targeted nucleic acid-binding analyte (13200), such as a nucleic acid-binding protein in contact with a nucleic acid, such as mRNA (13191), are presented in FIGS. 13A-13B. FIG. 13A depicts the step of contacting a biological sample including a plurality of nucleic acids with a first probe, wherein a first probe as depicted in FIG. 12A. FIG. 13B depicts modifying a 3′ and/or 5′ end of the nucleic acids in the sample by functionalizing with a moiety (★) for embedding the nucleic acids within a matrix, such as a gel.

FIG. 14A is a schematic diagram depicting embedding the probes and nucleic acid within a permeable matrix ( [symbol] ), such as a gel. FIG. 14B depicts permeabilization of the biological sample by removal of protein components.

FIG. 15A is a schematic diagram depicting contacting the permeabilized sample embedded within the matrix with an exemplary “second” nucleic acid capture probe having a nucleic acid capture agent (131020), including a nucleic acid-binding domain (13170) and a capture domain binding sequence (13180), as well as a first probe docking domain (13160). A splint oligonucleotide (131030) is also depicted, including a domain having a sequence complementary to the probe docking sites of the first probe (13140) and second probe (13150), to provide a splint to facilitate combining (e.g., ligation) of the first (131010) and second probe (131020). FIG. 15B is a schematic diagram depicting the hybridization of both the first and second probes via the splint oligonucleotide to form a chimeric “combined” probe (131240) that includes the specific capture domain (132001), the capture domain identification barcode (13110) and the nucleic acid binding site (13170) together with a capture domain binding sequence (13180). FIG. 15C is a schematic diagram depicting the step of contacting a biological sample including a plurality of nucleic acids with a first probe, wherein a first probe as depicted in FIG. 12E. Binding of the capture agent (132001) to a specific target protein is succeeded by the steps of embedding and permeabilizing the sample, followed by hybridization of the nucleic acid binding site (13170) to the nucleic acid (131040). A schematic diagram depicting the combined probe that would be created by permeabilizing the gel depicted in FIG. 15B with the target nucleic acid (13190) still bound (131240) is depicted in FIG. 15D, and with the target nucleic acid removed (131250) is depicted in FIG. 15E.

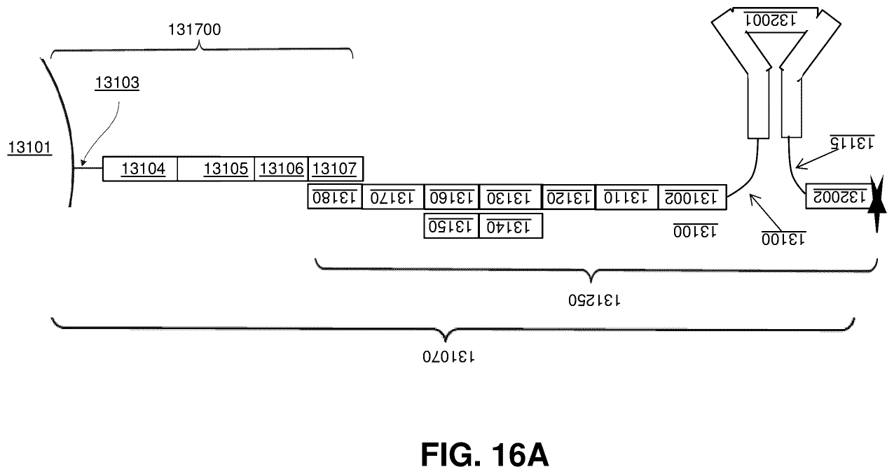

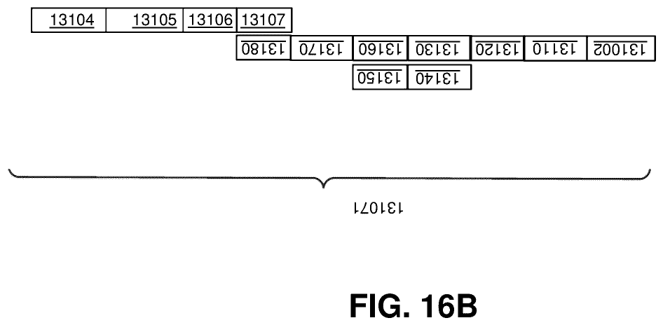

A schematic diagram depicting the capture of a combined probe (131240) of FIG. 15E by a capture probe bound to a substrate (131700) to form a “captured probe” (131070) is depicted in FIG. 16A. A schematic diagram depicting the minimal captured probe (131071) amenable for sequence analysis, resulting from cleavage of the linkers connecting the capture agent and substrate is depicted in (FIG. 16B).

### A. Features of First and Second Probes and Splint Oligonucleotides

Probes having one or more of features (i)-(ix), above are described. As discussed above, methods for enhanced spatial profiling of interactions between nucleic acids and nucleic acid-binding proteins employ between one and two types of functional probes, optionally together with one or more splint oligonucleotides, together with capture probes including a spatial barcode and a capture domain (see FIGS. 12A-12E, 13A-13B, 14A-14B, 15A-15E and 16A-16B), and optionally one or more additional functional elements in one or more of the probes, such as sequencing primer sites, etc.

When the methods include a first and a second probe, a first probe can be configured to include an analyte (e.g., a nucleic acid-binding protein) capture region, optionally associated with an oligonucleotide via a linker such as a cleavable linker, optionally a target identification sequence for identification of the target protein, and a second probe docking sequence that can bind to a corresponding docking sequence of a second probe; and a second probe can be configured to include a target nucleic acid capture domain (i.e., region complementary to target nucleic acid), a first probe docking sequence that can bind (e.g., hybridize) to a corresponding docking sequence of a second probe or a splint oligonucleotide, and a capture domain binding sequence that can bind (e.g., hybridize) to a capture domain of a capture probe. The chimeric (i.e., combined and/or ligated first and second) probe typically include at least a capture domain binding sequence that can bind to a capture domain of a capture probe.

When the methods include a first and a second probe and a splint oligonucleotide the first probe can be configured to include a target protein capture agent associated with an oligonucleotide optionally via a linker such as a cleavable linker, optionally a target identification sequence for identification of the target protein; and a first splint docking sequence that can hybridize to a complementary docking sequence of a splint oligonucleotide; the second probe can be configured to include a target nucleic acid capture domain, a second splint docking sequence that can hybridize to a complementary docking sequence of a splint oligonucleotide, and a capture domain binding sequence that can bind to a capture domain of a capture probe. The “splint” oligonucleotide can be configured to include (i) a region that is complementary to 5′ region of the first probe and (ii) a region that is complementary to 3′ region of the second probe, such that both the first and second probes can hybridize to the splint oligonucleotide to form a combined probe. In some forms, one or more functional domain sequences, or a complement thereof is also included within the splint oligonucleotide. Each of the functional elements of the oligonucleotide(s) for the methods is described in more detail, below.

In some forms, the first oligonucleotide is bound (e.g., conjugated or otherwise attached using any of the methods described herein) to a protein analyte capture agent. For example, the first probe can include an analyte capture agent, such as a protein binding moiety covalently linked to a single-stranded oligonucleotide. In some forms, the single-stranded oligonucleotide of the first probe is bound to the capture agent via its 5′ end. In some forms, the first probe includes a free 3′ end. In other forms, the single-stranded oligonucleotide of the first probe is bound to the capture agent via its 3′ end. In some forms, the first probe includes a phosphate at its free 5′ end. The first probe is functionalized with a moiety for gel embedding. In some forms, the first probe is functionalized with the moiety for gel embedding at its 5′ end. In some forms, the first probe is functionalized with the moiety for gel embedding at its 3′ end. In some forms, the first probe is functionalized with the moiety for gel embedding via functionalizing of the capture agent. In some forms, the first probe is functionalized with the moiety for gel embedding via functionalizing of an oligonucleotide tag, for example, a tag that is hybridized to the same end of the probe that is associated with the capture agent.

In some forms, the first probe is about 10 to about 150 nucleotides (e.g., about 10, about 20, about 30, about 40, about 50, about 60, about 70, about 80, about 90, about 100, about 110, about 120, about 130, about 140, about 150, or 200, 300 or 400 nucleotides) in length. In some forms, the first probe is a DNA molecule including DNA nucleotides (e.g., adenine (A), thymine (T), guanine (G), and cytosine (C)).

In some forms, the second probe includes a free 3′ end. In some forms, the second probe includes a nucleic acid-binding moiety. In some forms, the second probe includes a phosphate at its free 5′ end. In some forms, the second probe is about 10 to about 150 nucleotides (e.g., about 10, about 20, about 30, about 40, about 50, about 60, about 70, about 80, about 90, about 100, about 110, about 120, about 130, about 140, about 150, or 200, 300 or 400 nucleotides) in length. In some instances, the second probe is a DNA molecule including DNA nucleotides (e.g., adenine (A), thymine (T), guanine (G), and cytosine (C)).

In some forms, the splint oligonucleotide includes a free 3′ end and/or a free 5′. In some forms, the splint oligonucleotide includes a phosphate at its free 5′ end. In some forms, the splint oligonucleotide is about 10 to about 150 nucleotides (e.g., about 10, about 20, about 30, about 40, about 50, about 60, about 70, about 80, about 90, about 100, about 110, about 120, about 130, about 140, about 150, or 200, 300 or 400 nucleotides) in length. In some instances, the splint oligonucleotide is a DNA molecule including DNA nucleotides (e.g., adenine (A), thymine (T), guanine (G), and cytosine (C)).

In some forms, the size and/or sequence of a first-probe docking sequence, and/or a second-probe docking sequence, and/or a first splint oligonucleotide are configured so that combining the first probe and hybridized second probe will only occur when the target protein is bound directly to the nucleic acid. In some forms, the size and/or sequence of a first splint oligonucleotide is configured so that combining a first probe and hybridized second probe will only occur when the target protein interacts directly with the nucleic acid. An exemplary inter-molecular distance of interaction between a target protein that interacts directly with a nucleic acid is between 1 Å and 6 Å, inclusive. In an exemplary from, a distance of an interaction between a between a target protein and a nucleic acid with which it interacts directly is between 3 Å and 5 Å, inclusive for example, approximately 3 Å, 3.5 Å, 4 Å, 4.5 Å, or 5 Å. In some forms, the first probe includes a first barcode that is used to identify the capture agent and/or protein analyte to which it is targeted. In some forms, when the capture agent is or includes nucleic acids, such as an RNA aptamer, the first barcode is part or the capture agent.

The first probe includes a first docking sequence that enables the first probe to hybridize to a splint oligonucleotide. In some forms, a bridge sequence can include a sequence that is, or is complementary to one or more additional functional domain sequences. In some forms, the first docking sequence of the first oligonucleotide has at least 80%, at least 85%, at least 90%, at least 91%, at least 92%, at least 93%, at least 94%, at least 95%, at least 96%, at least 97%, at least 98%, or at least 99% sequence identity to a sequence that is 100% complementary of a splint oligonucleotide.

The second probe includes a second docking sequence that enables the second probe to hybridize to a splint oligonucleotide. In some forms, a bridge sequence can include a sequence that is, or is complementary to one or more additional functional domain sequences. In some forms, the first docking sequence of the first oligonucleotide has at least 80%, at least 85%, at least 90%, at least 91%, at least 92%, at least 93%, at least 94%, at least 95%, at least 96%, at least 97%, at least 98%, or at least 99% sequence identity to a sequence that is 100% complementary of a splint oligonucleotide.

Schematic diagrams depicting an exemplary arrangement of first probes configured for enhanced spatial profiling of interactions between nucleic acids and nucleic acid-binding proteins are depicted in FIGS. 12A-12E.

### 1. Target Binding Domains

The described methods require binding of a probe or probe set (i.e., a first probe and second probe) to a target nucleic acid-binding analyte and a target nucleic acid that interacts with the nucleic acid-binding analyte in the biological sample. Typically, the first probe includes an analyte capture agent that binds to a target nucleic acid-binding analyte (e.g., a nucleic acid binding protein). Typically, the second probe first probe includes a domain that binds to a target nucleic acid.

The compositions of first probes include oligonucleotides having at least one analyte capture agent that specifically and selectively binds to a non-nucleic acid analyte within a biological sample. Examples of non-nucleic acid analytes include, but are not limited to, nucleic acid binding proteins, such as DNA binding proteins and RNA-binding proteins, which physiologically bind to DNA (e.g., genomic DNA, cDNA) and RNA, including coding and non-coding RNA (e.g., mRNA, rRNA, tRNA, ncRNA), respectively. The term “analyte capture agent”, as used herein refers to a capture agent that specifically and selectively binds to a non-nucleic acid analyte within a biological sample from a subject. The compositions of first probes typically-include one analyte capture agent. In some forms, a single analyte capture agent can be specific to a single analyte. On the other hand, in some forms, an analyte capture agent can be designed to recognize and bind to a conserved region(s) that is, for example, present at the surface of a multiplicity of analytes. Thus, in some forms, an analyte capture agent binds to similar analytes in a biological sample (e.g., to detect conserved or similar analytes) or in different biological samples (e.g., across different species).

The compositions of second probe typically include one or more nucleic acid sequences (also referred to as binding domains) that hybridize with a region of a target nucleic acid that is specific to the target (e.g., compared to the entire genome) via interaction with the target. In some forms, sets of second probes are designed to include binding domains that collectively target all or nearly all of a genome (e.g., human genome). In forms, where the sets of second probes are designed to include binding domains that cover an entire genome (e.g., the human genome), the methods disclosed herein can detect interactions with target nucleic acids in an unbiased manner. In some forms, a target nucleic acid binding domain is designed to cover one target nucleic acid (e.g., transcript). In some forms, more than one target nucleic acid binding domain (e.g., a multiplicity of species of second probes) are designed to cover one target nucleic acid (e.g., transcript). For example, at least two, three, four, five, six, seven, eight, nine, ten, or more species of second probes each having a single, specific target nucleic acid binding domain can be used to hybridize to a single target nucleic acid. Factors to consider when designing target nucleic acid binding domains include presence of variants (e.g., SNPs, mutations) or multiple isoforms expressed by a single gene. In some forms, the second probe, or set thereof does not bind or hybridize to the entire target nucleic acid (e.g., a transcript), but instead the second probe binds or hybridizes to a discrete portion of the entire target nucleic acid (e.g., transcript). In some forms, a set includes about 5000, 10,000, 15,000, 20,000, or more second probe each having a single, specific target nucleic acid binding domain that is used in the methods described herein. In some forms, about 20,000 second probes each having a single, specific analyte binding domain are used in the methods described herein.

### (a.) Protein Binding Moieties

In some forms, the target nucleic acid-binding analyte is a protein, such as a nucleic acid-binding protein. In some forms, the nucleic acid-binding protein is a DNA-binding protein, or an RNA binding protein.

Therefore, in some forms, the first probe includes at least one analyte capture agent that is a protein binding moiety configured to bind selectively or specifically to a nucleic acid binding protein. In some forms, the protein binding moiety binds to an intracellular protein. For example, nucleic acid binding protein analytes can be derived from cytosol, from cell nuclei, from mitochondria, from microsomes, and more generally, from any other compartment, organelle, or portion of a cell.

In some forms, the protein binding moiety binds to a nucleic acid binding protein analyte present on the surface of or outside a cell. Protein analytes present on the surface of or outside a cell can include without limitation, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, and nucleic acid binding proteins having post translational modification (e.g., phosphorylation, glycosylation, ubiquitination, nitrosylation, methylation, acetylation or lipidation) state of a cell surface protein, a gap junction, and an adherens junction. In some forms, the protein binding moieties are capable of binding to cell surface analytes that are post-translationally modified.

### (i.) Antibodies

In some forms, the first probe includes a capture agent that is an antibody, or an antigen binding fragment or variant thereof. For example, in some forms the antibody is, without limitation, a monoclonal antibody, recombinant antibody, synthetic antibody, a single domain antibody, a single-chain variable fragment (scFv), and or an antigen-binding fragment (Fab).

In some forms, the methods include a secondary antibody that binds to a primary antibody. For example, in some forms, the first antibody binds to a first protein, and a secondary antibody binds to the first antibody. In some forms, multiple secondary antibodies can bind to a first antibody, allowing the detection of an analyte to be amplified. In some forms, a first probe as disclosed herein is bound to a secondary antibody, and the first probe can be used in a proximity ligation reaction disclosed herein.

### (ii.) Other Binding Agents

In some forms, the first probe includes a capture agent that is a non-protein. For example, in some forms, the capture agent is an aptamer.

Aptamers are nucleic acid macromolecules that bind to molecular targets, including proteins, with high affinity and specificity. Aptamers are typically from 15 to 40 nucleotides in length and can be composed of DNA, RNA, or nucleotides with a chemically modified sugar backbone (i.e., 2′-fluoro, 2′-O-methyl, phosphorothioate). Complementary base pairing defines aptamer secondary structure, consisting primarily of short helical arms and single-stranded loops. Stable tertiary structure, resulting from combinations of these secondary structures, allows aptamers to bind to targets via van der Waals, hydrogen bonding, and electrostatic interactions.

There exist a wide variety of aptamers that have been developed owing to their excellent properties compared to conventional antibodies: smaller physical size and lower immunogenicity and toxicity.

The huge number of possible tertiary structures allows aptamers to bind with high affinity via van der Waals, hydrogen bonding, and electrostatic interactions, to most small-molecule, peptide, or protein targets, with KD values ranging from 10 pM to 10 nM for proteins. Aptamers can recognize their targets with great specificity. For instance, an aptamer to bFGF (FGF-2) binds with up to 20,000-fold greater affinity to bFGF than it does to its closely related fibroblast growth factor (FGF)-1, -4, -5, -6, and -7 homologues. Other aptamers distinguish between closely related members of a protein family, or between different functional or conformational states of the same protein.

In some forms, aptamers for use as protein binding moieties in the described methods are prepared according to a process of in vitro selection, for example, according to the “SELEX” (Systematic Evolution of Ligands by Exponential enrichment) method. In some forms, the aptamers are isolated by various SELEX methods. e.g., that involve an iterative process of binding, partitioning, and amplifying novel nucleic acids from a combinatorial pool of up to 10¹⁶ variants. Therefore, in some forms, the methods include one or more steps of SELEX methodologies, i.e., by manually at the bench top or by automated systems. Typically, individual high-affinity aptamers are isolated after final refinement and optimization steps. Cloned aptamers are then screened for functional activity by, for example, their ability to modulate enzyme activity or their ability to neutralize their targets in cell-based assays. After the initial screen, it is usually desirable to determine the minimum sequence that still allows specific aptamer-target binding and efficacy. In some form, this process, minimization, is performed by mapping the critical parts of an aptamer via site-directed mutagenesis or chemical protection assays and eliminating superfluous regions. In some forms, minimization leads to increasing the affinity of the aptamer for the target, possibly due to a decrease in competing, nonbinding conformations. Other optimization steps include increasing the in vivo stability of an aptamer by substituting 2′-OHs with 2′-fluoro or 2′-O-methyl groups and chemically “capping” 5′ and 3′ termini.

In other forms, the methods provide aptamers having affinity for a selected target, developed using an expanded DNA alphabet to make more complex aptamers. An exemplary aptamer has the nucleic acid sequence:
5′-AGAGAGCGTCGTGTGGA-N25-TGAGGAGGTGCGCAAGT-3 (SEQ ID NO:1), wherein “N25” is a nucleotide sequence of 25 “N” nucleotides, wherein “N” is independently any nucleotide (e.g., A, G, T, C, etc.). Exemplary aptamers and methods for identifying and preparing aptamers are known in the art, for example, as described in Pendergrast, et al., “Nucleic Acid Aptamers for Target Validation and Therapeutic Applications”, J Biomol Tech. 2005 September; 16 (3): 224-234; Biondi, et al., “Laboratory evolution of artificially expanded DNA gives redesignable aptamers that target the toxic form of anthrax protective antigen”, Nucleic Acids Res. 2016 Nov. 16; 44(20): 9565-9577. and Popovic, et al., “Time-dependent regulation of cytokine production by RNA binding proteins defines T cell effector function”, Cell reports, v.42 (5), 112419, 2023, Butter, et al., “Unbiased RNA-protein interaction screen by quantitative proteomics”, PNAS, 106 (26) 10626-10631 (2009), doi.org/10.1073/pnas.081209910 the contents of each of which including all supplemental materials are hereby specifically incorporated by reference in their entireties.

In some forms, the capture agent is a DNA aptamer. In some forms, the aptamer is a single-stranded DNA molecule. In other forms, the aptamer is an RNA aptamer. In some forms, the aptamer is a synthetic aptamer. In some forms, the aptamer is a single-stranded RNA aptamer. In some forms, the aptamer is an aptamer that binds into its target by folding into tertiary structures. In some instances, the aptamer includes a sequence that can be used as an identification tag for the methods disclosed herein.

### (iii.) Linker Domains

In some forms, the first probe, or second probe, or capture probe includes one or more linkers. In some forms, a capture domain, such as a protein binding moiety is attached to a first probe by a linker. In some forms, the linker is a cleavable linker.

As used herein, a “linker” or “linker sequence” can refer to one or more nucleic acid sequences that are disposed between functional “domains” of sequences. In some forms, a linker includes a sequence that is not substantially complementary to either the sequence of a target nucleic acid or to a first or second probe docking sequence, or to a capture domain binding sequence, or to a capture domain of a capture probe. In some forms, the linker sequence includes ribonucleotides, deoxyribonucleotides, and/or synthetic nucleotides, where the sequence within the linker is not substantially complementary to either the sequence of a target nucleic acid or to a first or second probe docking sequence, or to a capture domain binding sequence, or to a capture domain of a capture probe.

In some forms the linker sequence includes a total of about 10 nucleotides to about 100 nucleotides, inclusive, or any of the subranges described herein. In some forms, a linker sequence includes a barcode sequence that serves as a proxy for identifying a targeted analyte and/or the capture probe. In some forms, the barcode sequence is a sequence that is at least 70% identical (e.g., at least 75% identical, at least 80% identical, at least 85% identical, at least 90% identical, at least 95% identical, or at least 99% identical) to a sequence in a targeted analyte. In some forms where a linker sequence includes a barcode sequence, the barcode sequence is located 5′ to the linker sequence. In some forms where a linker sequence includes a barcode sequence, the barcode sequence is located 3′ to the linker sequence. In some forms, the barcode sequence is disposed between two linker sequences. In such cases, the two linker sequences flanking the barcode sequence can be considered to be a part of the same linker sequence. Exemplary, cleavable linkers include a photocleavable linker, UV-cleavable linker, or an enzyme-cleavable linker.

### (b.) Nucleic Acid Binding Domains

In some forms, the first probe or second probe include a nucleic acid binding domain. For example, in some forms a second probe includes at least one nucleic acid binding domain configured to selectively and specifically hybridize with a target nucleic acid.

Typically, the nucleic acid binding domain includes a sequence complementary to a sequence of a target nucleic acid analyte. In some forms, a nucleic acid binding domain is configured to selectively and specifically hybridize with a target nucleic acid includes a poly(A) sequence. In some forms, a nucleic acid binding domain includes a poly-uridine sequence. In some forms where the target nucleic acid is an mRNA, and where all or part of the second probe includes deoxyribonucleotides, hybridization of the oligonucleotide to the mRNA molecule results in a DNA:RNA hybrid. In some forms, the second probe includes only deoxyribonucleotides and hybridization of the probe to the mRNA molecule results in a DNA:RNA hybrid.

Generally, the nucleic acid binding domain includes a sequence that is substantially complementary to a target sequence in the target nucleic acid. In some forms, the sequence that is substantially complementary to the target sequence in the targeted nucleic acid is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the target sequence in the target nucleic acid.

In some forms, nucleic acid binding domain is or includes one or more nucleic acid sequences from about four to about four hundred nucleotides, inclusive, (e.g., 4-100, or 4-50 nucleotides, inclusive) that are substantially complementary to one or more sequences of a targeted nucleic acid. In some forms the nucleic acid binding domain is a structured nucleic acid, such as an aptamer. Exemplary nucleic acid binding domains include from about 10 nucleotides to about 100 nucleotides in length, inclusive (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides, inclusive).

### (i.) RNA Targeting Sequences

In some forms, the nucleic acid analyte binding domain is configured to bind an RNA target, such as mRNA, for the spatial analysis of interactions between RNAs and RNA-binding proteins. Targeted RNA capture by a second probe according to the disclosed methods allows for spatial analysis of interactions between a subset of targeted RNA analytes from the entire transcriptome and RNA-binding analytes, such as RNA binding proteins. Therefore, in some forms, the nucleic acid binding domain is configured to bind a subset of analytes including an individual target RNA. In some forms, the targeted subset of analytes includes two or more targeted RNAs. In some forms, the targeted subset of analytes includes one or more mRNAs transcribed by one or more targeted genes. In some forms, the targeted subset of analytes includes one or more mRNA splice variants of one or more targeted genes. In some forms, the targeted subset of analytes includes non-polyadenylated RNAs in a biological sample. In some forms, the targeted subset of analytes includes detection of mRNAs having one or more single nucleotide polymorphisms (SNPs) in a biological sample.

In some forms, the nucleic acid binding domain is configured to bind a subset of analytes including mRNAs that mediate expression of a set of genes of interest. In some forms, the targeted subset of analytes includes mRNAs that share identical or substantially similar sequences, which mRNAs are translated into polypeptides having similar functional groups or protein domains. In some forms, the targeted subset of analytes includes mRNAs that do not share identical or substantially similar sequences, which mRNAs are translated into proteins that do not share similar functional groups or protein domains. In some forms, the targeted subset of analytes includes mRNAs that are translated into proteins that function in the same or similar biological pathways. In some forms, the biological pathways are associated with a pathologic disease.

In some forms, selective binding to one or more target nucleic acids by a second probe according to the disclosed methods employs nucleic acid analyte binding domains configured to bind genes that are overexpressed or under-expressed in cancer. In some forms, the targeted subset of analytes includes 2, 3, 4, 5, 6, 7, 8, 9, 10, 11, 12, 5 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1,000 analytes.

In some forms, the methods disclosed herein can detect interactions between one or more RNA-binding analytes and at least 5,000, 10,000, 15,000, 20,000, or more different RNAs. In some forms, the interactions detected by the methods provided herein includes interactions involving RNAs that include a large proportion of the transcriptome of one or more cells. For example, in some forms, the interactions detected by the methods provided herein include a subset of RNAs that represent at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

### (c.) Additional Functions/Elements of Analyte Binding Domains

In some forms, the sequence of a capture agent or a nucleic acid binding domain includes a sequence that performs one or more additional functions. For example, in some forms, the sequence of a capture agent that is or includes a nucleic acid, such as an aptamer, can include a functional domain. An exemplary functional domain includes a capture agent identification motif, for example, a sequence barcode for identification of the capture agent and/or the targeted nucleic acid binding analyte.

### 2. Functional Group(s) for Gel Embedding

Functional group(s) for gel embedding are described. The first probe includes one or more functional group(s) for gel embedding. Typically, the one or more functional group(s) for gel embedding is associated with a 5′ end of the second oligonucleotide. In other forms, the one or more functional group(s) for gel embedding is associated with a 3′ end of the second oligonucleotide. In some forms, the functional group(s) for gel embedding include one species of functional group. In other forms, the functional group(s) for gel embedding includes more than one species of functional group. In some forms, the functional group(s) for gel embedding includes acrydite.

In some forms, the matrix is a hydrogel matrix. In some forms, the tethering includes tethering a 3′ end of the target nucleic acid to the matrix. In some forms, the tethering includes tethering a 5′ end of the target nucleic acid to the matrix. In some forms, the tethering includes enzymatic tethering. In some forms, the tethering includes non-enzymatic tethering. In some forms, the tethering includes a periodate oxidation reaction or a reaction with boronic acid.

In some forms, the tethering includes tethering the target nucleic acid to the matrix via a linker. In some forms, the linker includes a cleavable linker. In some forms, the cleavable linker includes a disulfide bond. In some forms, the matrix is formed using N,N′-Bis(acryloyl) cystamine (BAC) as a crosslinker. In some forms, the linker is a photocleavable linker.

In some forms, the releasing includes contacting the matrix with a chemical agent configured to cleave a cleavable linker between the target nucleic acid and the matrix. In some forms, the releasing includes illuminating the matrix with light, thereby cleaving a photocleavable linker between the target nucleic acid and the matrix. In some forms, the releasing includes altering the pH of a solution in contact with the matrix, thereby releasing the tethered target nucleic acid.

In some forms, the target nucleic acid is tethered to the matrix via a boronate ester bond. In some forms, the boronate ester bond is formed between a boronic acid moiety and 3′ diols of the target nucleic acid which is an RNA. In some forms, the matrix includes a boronic acid-based hydrogel matrix. In some forms, the releasing includes exposing the matrix to heat, wherein the matrix is a hydrogel matrix including poly(N-isopropylacrylamide), thereby releasing the tethered target nucleic acid.

### 3. Capture Probe Binding Sequence

The first or second probes collectively include a probe capture domain binding sequence that hybridizes to a capture domain of a capture probe.

Typically, the probe capture domain binding sequence is a nucleic acid sequence that is complementary to a sequence of a capture probe having a defined sequence.

In some forms, a combined first and second probe, or ligation product thereof includes from 5′ to 3′: a sequence that is substantially complementary to a sequence of a capture domain of a capture probe. In other forms, a combined first and second probe, or ligation product thereof includes from 3′ to 5′: a sequence that is substantially complementary to a sequence of a capture domain.

In some forms, the sequence of a combined first and second probe, or ligation product thereof that is substantially complementary to the capture domain sequence in the capture probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the capture domain sequence of the capture probe.

In some forms, the capture domain binding sequence that is substantially complementary to the capture domain sequence of a capture probe includes a sequence that is about four nucleotides to about thirty nucleotides, inclusive, or about 5 nucleotides to about 50 nucleotides in length, inclusive, (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some forms, a second probe includes a complete capture domain binding sequence that is substantially complementary to the capture domain sequence in the capture probe; the capture domain-binding sequence is unaltered upon formation of a combined probe/ligation product thereof.

In some forms, a single “bi-specific” probe includes a complete capture domain binding sequence that is substantially complementary to the capture domain sequence in the capture probe.

### 4. Barcodes

In some forms, one or more of the first, or second probe or splint oligonucleotide include one or more sequence barcodes. For example, in some forms, the first probe includes a first barcode that is a molecular identifier of the protein that is bound by the capture agent/protein binding moiety. Typically the first barcode is specific to a protein and/or a binding moiety. In some forms, the first barcode is distinct from any other barcodes. In exemplary forms, the first barcode is a nucleic acid sequence of from about four to about twenty nucleic nucleotides, inclusive.

### 5. Adducts, Extensions and Functional Domains

In some forms, the first and/or second probe(s) and/or splint oligonucleotide include one or more additional functional elements, for example, a domain that implements a specific function or set of functions. See, for example, 131002 and 13120 in FIGS. 12A-12E. Exemplary functional domains that can be included with one or more of the first or second probes include a nucleic acid sequence that can include all or a part of sequencer specific flow cell attachment sequence (e.g., a P5 or P7 sequence), all or a part of a sequencing primer sequence, (e.g., a R1 primer binding site, a R2 primer binding site), or combinations thereof. In some forms, an oligonucleotide can also include a functional sequence that is a unique molecular identifier (UMI) sequence.

As used herein, an “extended probe” refers to a first or second probe having additional nucleotides added to the terminus (e.g., 3′ or 5′ end) of the probe thereby extending the overall length of the probe. For example, an “extended 3′ end” indicates additional nucleotides were added to the most 3′ nucleotide of the probe to extend the length of the probe, for example, by polymerization reactions used to extend nucleic acid molecules including templated polymerization catalyzed by a polymerase (e.g., a DNA polymerase or a reverse transcriptase). In some forms, the methods extend a combined probe that includes the first and second probe or a ligation product thereof. In some forms, extending the probe includes adding to a 3′ end of a probe a nucleic acid sequence that is complementary to a nucleic acid sequence of an analyte or intermediate agent specifically bound to the analyte capture domain of the probe. In some forms, the probe is extended using reverse transcription. In some forms, the probe is extended using one or more DNA polymerases. In some forms, the extended probe includes the sequence of a captured nucleic acid analyte and the sequence of capture domain binding sequence.

### 6. Exemplary First Probe Configuration

Compositions of a first probe for use according to the described methods are provided. Typically, the first probe includes a target protein-binding moiety and an associated first oligonucleotide including a second-probe docking sequence and, optionally, a target protein identification barcode; and one or more functional group(s) for embedding within a permeable matrix, such as a gel. The functional group is located at or near the target protein binding moiety, such that the probe is immobilized at or near the site of binding the target protein, and the opposing end of the probe includes the second probe docking sequence. For example, in some forms, the functional group and the target protein binding moiety are at the 5′ end of the first oligonucleotide, and second probe docking sequence is located at the 3′ end. In other forms, the functional group and the target protein binding moiety are at 3′ end of the first oligonucleotide, and second probe docking sequence is located at the 5′ end.

In some forms of the described methods, the first oligonucleotide further includes a second oligonucleotide hybridized thereto, and whereby one or more functional group(s) for gel embedding is associated with the second oligonucleotide.

In some forms, the first probe further includes a second oligonucleotide “tag” hybridized to the target protein binding moiety, or to the first oligonucleotide in the region of the target protein binding moiety. Therefore, in some forms, the one or more functional group(s) for gel embedding is associated with a 5′ end of the second oligonucleotide. In other forms, the one or more functional group(s) for gel embedding is associated with a 3′ end of the second oligonucleotide. In some forms, the functional group(s) for gel embedding include one species of functional group. In other forms, the functional group(s) for gel embedding includes more than one species of functional group. In some forms, the functional group(s) for gel embedding includes acrydite.

In some forms, the target protein binding moiety includes a polypeptide. For example in some forms, the target protein binding moiety includes an immunoglobulin, or antigen-binding fragment thereof. An exemplary immunoglobulin is a monoclonal antibody, or a polyclonal antibody. In some forms, the step of binding a target-protein specific primary antibody to the target protein is carried out prior to a step of contacting the biological sample with a first probe, and in some forms, the target protein-binding moiety includes a moiety that binds to the primary antibody. In some forms, the target protein-binding moiety that binds to the primary antibody is a secondary antibody.

In some forms, the target protein-binding moiety and/or the functional group(s) for embedding the probe within a permeable matrix, such as a gel, is associated with the first oligonucleotide via a linker, such as a cleavable linker.

Exemplary cleavable linkers for use in the first probe include a photocleavable linker, UV-cleavable linker, or an enzyme-cleavable linker. In some forms, the linker is a single stranded or double stranded nucleic acid, for example, including a recognition sequence for one or more restriction enzymes.

In some forms, the first probe is a releasable nucleic acid probe, including: (i) a target protein-binding moiety and an associated first oligonucleotide including a non-embeddable second-probe docking sequence and, optionally, a target protein identification barcode; and (ii) one or more functional group(s) for gel embedding associated with the target protein-binding moiety or the end of the first oligonucleotide proximal thereto. The target protein-binding moiety is associated with the first oligonucleotide via a releasable linker, such as a photocleavable linker, UV-cleavable linker, or an enzyme-cleavable linker. In some forms, the first cleavable linker includes single stranded or double stranded nucleic acid, for example, including a recognition and/or cut sequence for one or more restriction enzyme. In some forms, the first oligonucleotide of the first probe includes a target protein identification barcode, and/or one or more sequences that constitute a functional domain. In some forms, the size of the first oligonucleotide of the first probe is between about 6 and about 100 nucleotides. Exemplary first probes are depicted as 131010 in FIGS. 12A-12D.

### 7. Exemplary Second Probe Configuration

Compositions of a second probe for use according to the described methods are provided. Typically, the second probe includes: a first-probe docking sequence; a region complementary to one or more nucleic acids in a biological sample; and a capture domain binding sequence. In some forms, the region complementary to one or more target nucleic acids includes a known, pre-determined sequence designed to specifically bind to one or more target nucleic acids in the sample. In other forms, the region complementary to one or more target nucleic acids is not known. For example, in some forms the region complementary to one or more nucleic acids includes a random sequence, for example, including from about 4 to about 16 nucleotides, inclusive.

Therefore, in some forms, a plurality of second probes embodies a library of regions of complementarity, for example, a library that constitutes the sequences complementary to all or part of the each of the nucleic acids within a biological sample, wherein the size of the second probe is between about 6 and about 100 nucleotides.

In some forms, the second probe includes two or more smaller probes, that hybridize together upon recognition of a target nucleic acid. For example, in some forms, the second is formed from a first and second RNA-Templated Ligation (RTL) oligonucleotides. Typically, when a second probe is formed from a first and second RTL oligonucleotides, methods of contacting the sample with a second probe includes contacting the sample with the first and second RTL oligonucleotides, each including a region complementary to the same nucleic acid, but wherein a first RTL oligonucleotide includes a second probe docking domain at 5′ or 3′ end, and wherein the second RTL oligonucleotide includes a capture probe binding sequence at the opposing (3′ or 5′) end, respectively, such that hybridization and ligation of the first and second RTL oligonucleotides to the same target nucleic acid provides an intact second probe.

An exemplary second probe is depicted as 131020 in FIG. 15A.

### 8. Exemplary Splint Oligo Configuration

Compositions of a splint oligonucleotide for use according to the described methods are provided. Typically, the splint oligonucleotide includes a region complementary to a first-probe docking sequence and to a second-probe docking sequence. In some forms, the splint oligonucleotide includes one or more additional nucleic acid residues between the sequence complementary to the first-probe docking sequence and the sequence complementary to the second-probe docking sequence. It is contemplated that the splint oligonucleotide provides a bridge between the respective ends of the first and second probes, such that the size of the splint oligonucleotide can determine the ability of the first and second probes to combine to form a chimeric combined probe. Therefore, in some forms, the size and/or sequence of the first-probe docking sequence, and/or second-probe docking sequence, and/or the first splint oligonucleotide are configured so that combining the first probe and second probe will only occur when the target protein is bound directly to the nucleic acid. In some forms, the size and/or sequence of the first splint oligonucleotide is configured so that combining the first probe and hybridized second probe will only occur when the target protein interacts directly with the nucleic acid. In some forms, the size of the first splint oligonucleotide is between about 6 and about 100 nucleotides, inclusive. In some forms, the distance between the target protein and protein-bound ribonucleic acid molecules is less than 0.8 nm. An exemplary second probe is depicted as 131030 in FIG. 15A.

### B. Capture Probes

Compositions of capture probes including a spatial barcode and a capture domain are utilized for the described methods.

Typically the capture probe is any molecule capable of capturing (directly or indirectly) the described combined probe (i.e., first and second probe(s) or ligation product thereof).

In some forms, the capture probe is a nucleic acid or a polypeptide that includes at least one capture domain. Typically, the capture probe includes a barcode (e.g., a spatial barcode and/or a unique molecular identifier (UMI)) and a capture domain). In some forms, a capture probe can include a cleavage domain and/or a functional domain (e.g., a primer-binding site, such as for next generation sequencing (NGS)). See, e.g., WO 2020/176788 and/or U.S. Patent Application Publication No. 2020/0277663. Generation of capture probes can be achieved by any appropriate method, including those described in WO 2020/176788 and/or U.S. Patent Application Publication No. 2020/0277663. In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., WO 2020/176788 and/or U.S. Patent Application Publication No. 2020/0277663 regarding extended capture probes).

Typically, for spatial array-based methods, a substrate functions as a support for direct or indirect attachment of capture probes to features of the array. A “feature” is an entity that acts as a support or repository for various molecular entities used in spatial analysis. In some forms, some or all of the features in an array are functionalized for analyte capture. Exemplary substrates are described in WO 2020/176788 and/or U.S. Patent Application Publication No. 2020/0277663. Exemplary features and geometric attributes of an array can be found in WO 2020/176788 and/or U.S. Patent Application Publication No. 2020/0277663. An exemplary capture probe (131700) configured to be immobilized on a substrate (13101) is depicted in FIG. 16A.

In some forms, the capture probe includes one or more additional functional elements, for example, a domain that implements a specific function or set of functions. See, for example, 13105 and 13106 in FIGS. 16A-16B. Exemplary functional domains that can be included with capture probes include a nucleic acid sequence that can include all or a part of sequencer specific flow cell attachment sequence (e.g., a P5 or P7 sequence), all or a part of a sequencing primer sequence, (e.g., a R1 primer binding site, a R2 primer binding site), or combinations thereof. In some forms, an oligonucleotide can also include a functional sequence that is a unique molecular identifier (UMI) sequence.

### 1. Capture Domain Sequence

The capture probe includes a capture domain that hybridizes to a capture domain binding sequence of a second probe or combined probe/ligation product thereof.

Typically, the capture domain is a nucleic acid sequence that is complementary to a capture domain binding sequence of a combined probe having a defined sequence. In some forms, a capture domain includes from 5′ to 3′: a sequence that is substantially complementary to a sequence of a capture domain binding sequence of a second probe or combined probe/ligation product thereof. In other forms, a capture domain includes from 3′ to 5′: a sequence that is substantially complementary to a sequence of a capture domain binding sequence of a second probe or combined probe/ligation product thereof.

In some forms, the sequence of a capture domain that is substantially complementary to the capture domain binding sequence of a second probe or combined probe/ligation product thereof is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the capture domain binding sequence of a second probe or combined probe/ligation product thereof.

In some forms, the capture domain sequence that is substantially complementary to the capture domain binding sequence of a second probe or combined probe/ligation product thereof includes a sequence that is about four nucleotides to about thirty nucleotides, inclusive, or about 5 nucleotides to about 50 nucleotides in length, inclusive, (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

### C. Additional Reagents

Any of the methods for enhanced spatial profiling of interactions between nucleic acids and nucleic acid-binding proteins provided herein can include one or more of the following reagents, such as biological samples, reagents for sample preparation, probe hybridization and sequence analysis.

### 1. Biological Samples

In some forms, a biological sample can be a tissue section. In some forms, the biological sample is a tissue sample. In some forms, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates. In some forms, the sample is a fresh frozen sample (e.g., tissue sample). In some forms, the sample (e.g., tissue sample) was previously frozen.

In some forms, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains).

In some forms, a biological sample can be fixed with a fixative selected from ethanol, methanol, acetone, formaldehyde, paraformaldehyde-Triton, glutaraldehyde, and combinations thereof. Thus, in some forms, the biological sample is a formalin-fixed paraffin embedded tissue sample, a paraformaldehyde fixed tissue sample, a methanol fixed tissue sample, or an acetone fixed tissue sample. In some instances, the biological sample is fixed using PAXgene. PAXgene is a formalin-free, non-cross-linking fixative that preserves morphology and biomolecules. It is a mixture of different alcohols, acid, and a soluble organic compound. Ergin B. et al., J Proteome Res. 2010 Oct. 1; 9(10):5188-96 describes the development of PAXgene. Kap M. et al., PLOS One.; 6(11):e27704 (2011) and Mathieson W. et al., Am J Clin Pathol.; 146 (1): 25-40 (2016) both describe and evaluate PAXgene for tissue fixation.

In some forms, a biological sample (e.g., a fixed and/or stained biological sample) can be imaged. Biological samples are also described in Section (I)(d) of WO 2020/176788 and/or U.S. Patent Application Publication No. 2020/0277663.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some forms, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some forms, the biological sample includes cancer or tumor cells. Cancer cells can be derived from solid tumors, hematological malignancies, cell lines, or obtained as circulating tumor cells. In some forms, the biological sample is a heterogenous sample. In some forms, the biological sample is a heterogenous sample that includes tumor or cancer cells and/or stromal cells.

In some forms, the cancer is breast cancer. In some forms, the breast cancer is triple positive breast cancer (TPBC). In some forms, the breast cancer is triple negative breast cancer (TNBC). In some forms, the cancer is colorectal cancer. In some forms, the cancer is ovarian cancer. In certain forms, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain forms, the cancer treated is desmoplastic melanoma, inflammatory breast cancer, thymoma, rectal cancer, anal cancer, or surgically treatable or non-surgically treatable brain stem glioma. In some forms, the subject is a human.

### (a.) Target Nucleic Acids

Compositions for use with the described methods can include one or more second probes configured to bind one or more classes of nucleic acids. Therefore the methods can provide enhanced spatial profiling of interactions involving any nucleic acid present in a biological sample and a binding partner. Exemplary classes of target nucleic acids include biological DNA, such as genomic DNA, and RNA, as well as synthetic DNA or synthetic RNA. Exemplary classes of target RNAs include small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (incRNA), and transfer RNA (tRNA). In certain forms, the target RNA is transcriptomic RNA, such as mRNA.

### 2. Permeable Matrices

Permeable matrices for encapsulating and embedding samples are provided. Typically, the permeable matrices are gels, such as hydrogels.

In some forms, the matrix is a hydrogel matrix. In some forms, the matrix is formed using N,N′-Bis(acryloyl) cystamine (BAC) as a crosslinker. In some forms, the linker is a photocleavable linker.

In some forms, the matrix includes a boronic acid-based hydrogel matrix. In some forms, the matrix is a gel that can be dissolved or disassembled by exposing the matrix to heat, wherein the matrix is a hydrogel matrix including poly(N-isopropylacrylamide.

In some forms, the biological sample is embedded in a matrix (e.g., a hydrogel matrix). Embedding the sample in a hydrogel matrix in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some forms, the hydrogel is formed such that the hydrogel is internalized within the biological sample. Biological samples can include analytes (e.g., protein, RNA, and/or DNA) embedded in a 3D matrix. In some forms, a 3D matrix may include a network of natural molecules and/or synthetic molecules that are chemically and/or enzymatically linked, e.g., by crosslinking. In some forms, a 3D matrix may include a synthetic polymer. In some forms, a 3D matrix includes a hydrogel.

In some forms, the biological sample is immobilized in the hydrogel via cross-linking of the polymer material that forms the hydrogel. Cross-linking can be performed chemically and/or photochemically, or alternatively by any other suitable hydrogel-formation method. In some forms, the biological sample is reversibly cross-linked prior to or during an assay disclosed herein.

In some forms, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some forms, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some forms, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample can vary. As one example, where the biological sample is a tissue section, the hydrogel-matrix can include a monomer solution and an ammonium persulfate (APS) initiator/tetramethylethylenediamine (TEMED) accelerator solution. As another example, where the biological sample contains cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional compositions for hydrogel embedding of biological samples are described for example in Chen et al., Science 347(6221):543-548, 2015, the entire contents of which are incorporated herein by reference.

In some forms, hydrogel subunits are infused into the biological sample, and polymerization of the hydrogel is initiated by an external or internal stimulus.

In some forms, hydrogel formation within a biological sample is permanent. For example, biological macromolecules can permanently adhere to the hydrogel allowing multiple rounds of interrogation. In some forms, hydrogel formation within a biological sample is reversible. In some forms, HTC reagents are added to the hydrogel before, contemporaneously with, and/or after polymerization. In some forms, a cell labeling agent is added to the hydrogel before, contemporaneously with, and/or after polymerization. In some forms, a cell-penetrating agent is added to the hydrogel before, contemporaneously with, and/or after polymerization.

### 3. Functional Group(s) for Embedding

Functional groups for embedding nucleic acids within a permeable matrix are provided. An exemplary functional group is acrydite.

In forms in which a hydrogel is formed within a biological sample, functionalization chemistry can be used. In some forms, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT.

In some forms, nucleic acids (e.g., RNA) in a biological sample embedded in a matrix can be tethered to the matrix, followed by clearing the biological sample to remove non-nucleic acid components. In some forms, the tethering is reversible. The nucleic acids can remain tethered during sample clearing, and the tethering can be reversed and/or the nucleic acids (or a proxy thereof) be released for subsequent analysis. In some forms, dissolvable gel formulations can be used, such that nucleic acids can remain tethered to the hydrogel during sample clearing, whereas the hydrogel can be dissolved to release the nucleic acids after sample clearing.

In some forms, RNA tethering to the matrix can use boronic acid moieties in the matrix to passively capture 3′ RNA ends that have been polished, where the tethering occurs when the pH is greater than the pKa of the boronic acid moiety (which induces the formation of boronate esters with 3′ RNA diols) and the tethering is reversed when the pH is below the pKa.

In some forms, a hydrogel including a boronic acid moiety can be used to tether an RNA of a sample (e.g., a cell or tissue sample embedded in the hydrogel) for subsequent removal of proteins (e.g., ribosome components) and lipids. A drop in pH below the pKa of the boronic acid moieties in the tethered sample can release the RNAs or other nucleic acid analyte to begin interfacing with the arrayed capture probes. In some forms, a slightly acidic buffer is used to un-tether the RNA, where the proximity of the array capture probes helps limit lateral diffusion through the hydrogels to mitigate mis-localization. In some forms, capacitance can be used to drive migration of the newly liberated RNA towards the array.

In some forms, a boronic acid-based hydrogel can be used as a sample hydrogel for embedding a sample. In some forms, boronic acids may be embedded within the sample hydrogel matrix (e.g., a hydrogel mesh), to passively capture 3′ RNA ends that have been polished. In some approaches, the tethering of the RNA target molecules occurs if the pH of the system is greater than the pKa of the boronic acid. In some forms, this may be due to the formation of boronate esters between the boronic acid groups of the sample hydrogel with 3′ RNA diols of the target RNA molecule. For forms, where the pH is below the pKa tethering may be revered and the tethered RNA target molecule can be released. In such an form, no separate linker molecule is required.

In some examples, pH triggered release of target molecules (e.g., target nucleic acid, target RNA, target RNA molecules) tethered to a hydrogel may be followed by diffusion of the target molecule to the array of capture probes. In some examples, capacitance may be utilized to limit diffusion in one direction (e.g., anisotropic diffusion), where released target molecules are directed toward the capture array, thereby increasing the efficiency of the capture process.

In some forms, a target RNA disclosed herein includes a 5′-phosphate group or a 5′-phosphate group modified with a leaving group, and the biological sample containing the target RNA can be contacted with an attachment agent including (i) at least one reactive moiety capable of reacting with at least one 5′-phosphate group of the target RNA or 5′-phosphate group of the RNA modified with a leaving group, and (ii) at least one attachment moiety capable of attaching covalently or noncovalently to a matrix-forming agent or a matrix formed by the matrix-forming agent. A covalent bond can be formed between the reactive moiety of the attachment agent and the target RNA, thereby 5′ tethering the target RNA to the matrix, such as a hydrogel matrix. In some forms, the biological sample is contacted with the matrix forming agent, and a three-dimensional polymerized matrix can be formed from the matrix-forming agent that permeates the sample, thereby embedding the biological sample and immobilizing the target RNA in the three-dimensional polymerized matrix.

In some forms, the attachment agent is a compound of Formula (I):

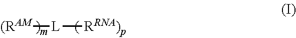

or a salt thereof, wherein each R^{RNA} is independently a reactive moiety capable of reacting with at least one 5′-phosphate group of the RNA or 5′-phosphate group of the RNA modified with a leaving group; each R^{AM} is independently an attachment moiety capable of attaching covalently or noncovalently to a matrix-forming agent; L is a bond or a linker moiety; m is an integer from 1 to 4; and p is an integer from 1 to 4.

In some forms, the attachment agent is of Formula (I-a):

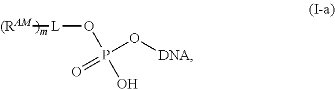

wherein DNA-1 includes a nucleic acid sequence of 7-15 nucleotide residues; each R^{AM} is independently an attachment moiety capable of attaching covalently or noncovalently to a matrix-forming agent; L is a bond or a linker moiety; and m is an integer from 1 to 4.

In some forms, the attachment agent is of Formula (I-b):

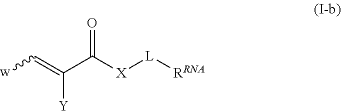

wherein R^{RNA} is independently a reactive moiety capable of reacting with at least one 5′-phosphate group of the RNA or 5′-phosphate group of the RNA modified with a leaving group; L is a bond or a linker moiety; W is independently H or C₁₋₆ alkyl; Y is H or C₁₋₆ alkyl; and X is NH, N(C₁₋₆ alkyl), or O.

### 4. Sample Permeabilization Reagents

In some forms, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Forms Section of WO 2020/176788 and/or U.S. Patent Application Publication No. 2020/0277663. In some forms, permeabilizing includes the use of an organic solvent, a detergent, an enzyme, or a combination thereof. In some forms, permeabilizing includes the use of an endopeptidase (e.g., pepsin or proteinase K), a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

### IV. Kits for Enhanced Spatial Profiling of Analyte Interactions

Provided herein are kits that include one or more compositions and/or reagents for enhanced spatial profiling of interactions between nucleic acids and nucleic acid-binding proteins in a biological sample.

Kits of components and/or reagents typically include one or more of:
(a) a plurality of first probes, wherein a first probe of the plurality of first probes includes: (i) a target protein-binding moiety and an associated first oligonucleotide including a non-embeddable second-probe docking sequence, and (ii) one or more functional group(s) for gel embedding; and
(b) instructions for performing the described methods for enhanced spatial profiling of interactions between nucleic acids and nucleic acid-binding proteins in a biological sample.

Exemplary instructions include one or more steps of:
(a) contacting a biological sample with the plurality of first probes;
(b) modifying a 3′ and/or 5′ end of the nucleic acids in the sample with one or more functional group(s) for gel embedding;
(c) embedding the biological sample in a permeable matrix by forming an interaction between the one or more functional group(s) and the matrix;
(d) hybridizing the plurality of second probes to the plurality of nucleic acids to form a plurality of hybridized second probes,
(e) combining the first probe and the hybridized second probe to form a combined probe including a capture domain binding sequence,
(f) releasing the combined probe from the functional group(s); and
(g) hybridizing the capture domain binding sequence of the combined probe to the capture domain of a capture probe.

In some forms, the first probe of the plurality of first probes includes (i) a target protein-binding moiety and an associated first oligonucleotide including a non-embeddable second-probe docking sequence and, optionally, a target protein identification barcode; and (ii) one or more functional group(s) for gel embedding associated with the target protein-binding moiety or the end of the first oligonucleotide proximal thereto. In some forms, the target protein-binding moiety is associated with the first oligonucleotide via a first cleavable linker. In some forms, the first cleavable linker is a photocleavable linker, UV-cleavable linker, or an enzyme-cleavable linker. In some forms, the cleavable linker is a single stranded or double stranded nucleic acid, for example, including a recognition and/or cut sequence for one or more restriction enzyme.

In some forms, the kit further includes one or more of:
(a) a plurality of second probes specific for a target nucleic acid, including (i) a first-probe docking sequence; and (ii) a region complementary to one or more ribonucleic acids of the plurality of nucleic acids; and/or
(b) a solvent suitable to solubilize unbound first probes and/or unbound second probes; and/or (c) one or more Polymerase enzymes; and/or
(d) one or more nuclease enzymes.

In some forms, the kit further includes one or more splint oligonucleotides. Exemplary splint oligonucleotides include (i) a sequence complementary to a docking sequence of a first-probe; and (ii) a sequence complementary to a docking sequence of a second-probe.

In some forms, the kit further includes a plurality of capture probes. Exemplary capture probes include (i) a spatial barcode; and (ii) a capture domain.

In some forms, the kit further includes one or more species of enzymes, such as enzymes needed for performing the methods according to instructions. Exemplary enzymes include a ligase, polymerase, permeabilization reagent, wash buffer, or a combination thereof.

In some forms, the kit includes a target protein binding moiety that binds to one or more proteins selected from the group including Hu-antigen R (HuR), heterogeneous nuclear ribonucleoprotein family (hnRNP), the arginine/serine-rich splicing factor protein family (SRSF), and RNA-binding motif (RBM) proteins. In some forms, the kit includes a target protein binding moiety that binds to the expression product of a gene selected from TERT, ZC3H12C, ZNF106, TLR3, PIWIL2, APC, EXO1, RNASE4, SRSF12, RBM11, LRRFIP1, OASL, SMAD4, DKC1, ANG, SRSF5, KHDRBS2, ARHGEF28, SIDT2, RBPMS, INTS10, SECISBP2L, FTO, EIF1B, RNASE1, DICER1, EZH2, LARS2, ZCCHC2, ZFP36, EXOSC7, NOVA1, MEX3A, HINT3, RBMS3, RNASE6, PATL2, BYSL, ESRP2, CNOT8, RPL22L1, POLR2H, RNASE3, ELAVL2, MRPS36, PPARGC1B, ADAD2, CPEB4, PTBP1, CTIF, HBS1L, PNRC2, MRPL12, TDRD9, DYNC1H1, ADARB2, PDCD4, TEP1, DDX24, GFM1, TARBP1, ELAC1, RPL22, PTBP2, TARS, RBM7, BRIX1, ENOX1, SRRM4, CWF19L2, ZFP36L1, CLK4, DUSIL, HABP4, METTL1 and XPO4 or a protein that interacts directly with one or more of these expression products.

In some forms, the kit includes a target protein binding moiety that binds to the expression product of a gene selected from RBM3, RBM4, RBM5, RBM6, RBM7, RBM8A, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM33, RBM34, RBM38, RBM39, RBM41, RBM42, RBM43, RBM44, RBM45, RBM46, RBM47, RBM48, RBM4B, RBMS1, RBMS2, RBMS3, RBMX, RBMX2, RBMXL1, RBMXL2, RBMXL3, RBMY1A1, RBMY1B, RBMY1C, RBMY1D, RBMY1E, RBMY1F, and RBMY1J, or a protein that interacts directly with one or more of these expression products.

In certain forms, the kit includes a target protein binding moiety that selectively binds to Human antigen R (HuR).

In some forms, the kit includes a nucleic acid capture probe that selectively binds mRNA encoding a protein selected from the group including c-Fos, c-Myc, p21, p27, cyclin A2, cyclin B1, cyclin E1, cyclin D1, OSM, eIF4E, EGF, VEGF, HIF-1α, cyclooxygenase-2 (COX-2), iNOS, TSP1, TGF-β, MKP-1, Mdm2, SIRT1, Bcl-2, Mcl-1, XIAP, Cyto c, uPA, uPAR, MMP-9, Snail, dCK, p53, pVHL, ARH1/DRAS3, BRCA1, Estrogen Receptor, Wnt5a, c-Fms, GATA3, GM-CSF, TNF-α, TM, RGS4, TLR4, IL-6, IL-8, IL-13, SMN, SH2D1A, NF1, PROX1, Eotaxin, ProTα, and IGF-1RA. In some forms, the kit includes a nucleic acid capture probe that selectively binds mRNA encoding to mRNA encoding the cyclooxygenase-2 (COX-2) protein, or a protein that interacts directly with one or more of these proteins. Therefore, in certain forms, the kit includes a target protein binding moiety that selectively binds to Human antigen R (HuR), and a nucleic acid capture probe that selectively binds mRNA encoding the cyclooxygenase-2 (COX-2) protein, or a protein that interacts directly with one or more of these proteins.

The disclosed methods and compositions can be further understood through the following numbered paragraphs.
1. A method of determining a location and/or abundance of an interaction between a target protein and a target nucleic acid in a biological sample, the method including:
(a) contacting a biological sample including a plurality of nucleic acids with a plurality of first probes, wherein a first probe of the plurality of first probes includes
   (i) a target protein-binding moiety and a first oligonucleotide including a second-probe docking sequence and, optionally, a target protein identification barcode, and
   (ii) one or more functional group(s), under conditions suitable for the target protein-binding moiety to bind to a target protein;
(b) modifying a 3′ and/or 5′ end of the plurality of nucleic acids in the biological sample with one or more functional group(s),
wherein the target nucleic acid is included in the plurality of nucleic acids;
(c) embedding the biological sample in a gel, including forming an interaction between the one or more functional group(s) and the gel;
(d) hybridizing a plurality of second probes to the plurality of nucleic acids to form a plurality of hybridized second probes,
wherein a hybridized second probe of the plurality of hybridized second probes includes
   (i) a first-probe docking sequence;
   (ii) a region complementary to the target nucleic acid; and
   (iii) a capture domain binding sequence;
(e) combining the first probe and the hybridized second probe to form a combined probe including the capture domain binding sequence,
(f) releasing the combined probe;
(g) hybridizing the capture domain binding sequence of the combined probe to a capture domain of a capture probe,
wherein the capture probe includes
   (i) a spatial barcode, and (ii) the capture domain; and
(h) determining
   (hi) the spatial barcode sequence or a complement thereof;
   (hii) all or a portion of the hybridized second probe sequence or a complement thereof; and/or
   (hiii) the sequence of the target protein identification barcode; and
(i) using the determined sequences of (hi), (hii) and optionally (hiii) to identify the location and/or abundance of the interaction between the target protein and target nucleic acid in the biological sample.
2. The method of paragraph 1, wherein the gel is a hydrogel.
3. The method of any one of paragraphs 1 or 2, wherein the target nucleic acid is DNA.
4. The method of any one of paragraphs 1 or 2, wherein the target nucleic acid is RNA.
5. The method of paragraph 4, wherein the RNA includes one or more selected from the group including small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (incRNA), and transfer RNA (tRNA).
6. The method of paragraph 5, wherein the RNA includes mRNA.
7. The method of any one of paragraphs 1-6, further including one or more steps of fragmenting the plurality of nucleic acids.
8. The method of paragraph 7, wherein the plurality of nucleic acids are fragmented prior to step (a).
9. The method of paragraph 8, wherein the fragmenting occurs after or during any one of steps (a), (b), (c), (d) or (e).
10. The method of paragraph 8 or 9, wherein the fragmenting includes contacting the biological sample with one or more nuclease enzymes.
11. The method of any one of paragraphs 1 to 10, further including permeabilizing the biological sample.
12. The method of paragraph 11, wherein the permeabilizing is performed during step (c), and/or during step (f).
13. The method of paragraph 12, wherein the permeabilizing includes contacting the biological sample with a permeabilization reagent.
14. The method of paragraph 13, wherein the gel includes the permeabilization reagent.
15. The method of paragraph 13 or 14, wherein the permeabilizing reagent is selected from an organic solvent, a detergent, and an enzyme, or a combination thereof.
16. The method of paragraph 15, wherein the permeabilization agent is selected from the group including an endopeptidase, a protease, sodium dodecyl sulfate (SDS), polyethylene glycol tert-octylphenyl ether, polysorbate 80, polysorbate 20, N-lauroylsarcosine sodium salt solution, and saponin.
17. The method of paragraph 16 wherein the permeabilization agent includes a protease.
18. The method of any one of paragraphs 1-17, wherein step (f) and/or step (g) further includes degrading or otherwise removing the target nucleic acid hybridized to the combined probe and/or releasing the combined probe from the functional group(s).
19. The method of paragraph 18, wherein the degrading or otherwise removing includes contacting the biological sample with a nuclease.
20. The method of paragraph 19, wherein the nuclease is a DNAse or an RNAse.
21. The method of paragraph 20, wherein the target nucleic acid is RNA and wherein the RNase is an RNase H.
22. The method of any one of paragraphs 1-21, wherein the first oligonucleotide includes the target protein identification barcode.
23. The method of any one of paragraphs 1-22, wherein the region complementary to the target nucleic acid includes a random sequence, optionally wherein the random sequence includes from about 4 to about 10 nucleotides, inclusive.
24. The method of paragraph 23, wherein the plurality of second probes includes a library of sequences complementary to all or part of each nucleic acid of the plurality of nucleic acids.
25. The method of any one of paragraphs 1-24, wherein the first-probe docking sequence is substantially complementary to the second-probe docking sequence, and wherein combining the first probe and the hybridized second probe includes hybridizing the first-probe docking sequence with the second-probe docking sequence.
26. The method of any one of paragraphs 1-25, wherein the first-probe docking sequence is not complementary to the second-probe docking sequence, and wherein combining the first probe and the hybridized second probe to form a combined probe including a capture domain binding sequence in step (e) includes use of a first splint oligonucleotide.
27. The method of paragraph 26, wherein the first splint oligonucleotide includes
   (i) a sequence complementary to the first-probe docking sequence; and
   (ii) a sequence complementary to the second-probe docking sequence.
28. The method of paragraph 27, wherein combining the first probe and the hybridized second probe includes hybridizing the first splint oligonucleotide to both the first-probe docking sequence and the second-probe docking sequence, and ligating the first probe and second hybridized probe together to form the combined probe, optionally wherein the ligating includes ligating the second-probe docking sequence of the first probe and the first-probe docking sequence of the second probe together, optionally wherein the ligating includes chemical ligation or enzymatic ligation.
29. The method of any one of paragraphs 26-28, wherein the first splint oligonucleotide includes one or more additional nucleic acid residues between the sequence complementary to the first-probe docking sequence and the sequence complementary to the second-probe docking sequence.
30. The method of any one of paragraphs 26 to 29, wherein the size and/or sequence of the first-probe docking sequence, and/or second-probe docking sequence, and/or the first splint oligonucleotide are configured so that combining the first probe and hybridized second probe in step (e) will preferably occur when the target protein is bound directly to the target nucleic acid.
31. The method of any one of paragraphs 26-30, wherein the size and/or sequence of the first splint oligonucleotide is configured so that combining the first probe and hybridized second probe in step (e) will preferably occur when the target protein interacts directly with the target nucleic acid.
32. The method of any one of paragraphs 26-31, wherein the size of the first splint oligonucleotide is between about 6 and about 100 nucleotides.
33. The method of any one of paragraphs 1-32, wherein the distance between the target protein and the target nucleic acid is less than 0.8 nm.
34. The method of any one of paragraphs 1-33, wherein the size of the first oligonucleotide is between about 6 and about 100 nucleotides, inclusive.
35. The method of any one of paragraphs 1-34, wherein the size of the second probe is between about 6 and about 100 nucleotides, inclusive.
36. The method of any one of paragraphs 1-35, wherein the hybridized second probe includes a first and a second RTL oligonucleotide, wherein each of the first and second RTL oligonucleotides includes a sequence complementary to the target nucleic acid.
37. The method of paragraph 36, wherein hybridizing a plurality of second probes to the plurality of nucleic acids in step (d) includes
(i) hybridizing the first RTL probe with the target nucleic acid;
(ii) hybridizing a second RTL probe with the target nucleic acid; and
(iii) ligating the first and second RTL probes together to form the hybridized second probe.
38. The method of any one of paragraphs 1-37, wherein the target protein-binding moiety is selected from an antibody or antigen-binding fragment thereof, an aptamer, a lectin, a small molecule, an enzyme, a nucleic acid and a target molecule-specific ligand.
39. The method of any one of paragraphs 1-38, wherein, wherein the target protein binding moiety includes a nucleic acid.
40. The method of any one of paragraphs 1-39, wherein the target protein binding moiety includes an aptamer.
41. The method of paragraph 39 or 40, wherein the sequence of the target protein binding moiety includes a target protein identification barcode.
42. The method of any one of paragraphs 1-38, wherein the target protein binding moiety includes a polypeptide.
43. The method of any one of paragraphs 1-38 or 42, wherein the target protein binding moiety includes an immunoglobulin, or antigen-binding fragment thereof.
44. The method of paragraph 43, wherein the immunoglobulin is a monoclonal antibody, or a polyclonal antibody.
45. The method of any one of paragraphs 1-38, or 43-44, further including a step of binding a target-protein specific primary antibody to the target protein prior to step (a).
46. The method of paragraph 45, wherein the target protein-binding moiety includes a moiety that binds to the primary antibody.
47. The method of paragraph 46, wherein the target protein-binding moiety that binds to the primary antibody includes a secondary antibody.
48. The method of any one of paragraphs 1-47, wherein the target protein-binding moiety is associated with the first oligonucleotide via a first cleavable linker.
49. The method of paragraph 48, wherein the first cleavable linker includes a photocleavable linker, UV-cleavable linker, or an enzyme-cleavable linker.
50. The method of paragraph 48 or 49, wherein the first cleavable linker is an enzyme-cleavable linker.
51. The method of any one of paragraphs 48-50, wherein the first cleavable linker includes a single stranded or double stranded nucleic acid.
52. The method of paragraph 51, wherein the single stranded or double stranded nucleic acid includes a recognition sequence for one or more restriction enzymes.
53. The method of any one of paragraphs 1-52, wherein the target protein-binding moiety and the first oligonucleotide including the second-probe docking sequence and, optionally, a target protein identification barcode in (ai), and the one or more functional group(s) in (aii) are associated via a second cleavable linker.
54. The method of paragraph 53, wherein the second cleavable linker includes a photocleavable linker, UV-cleavable linker, or an enzyme-cleavable linker.
55. The method of paragraph 54, wherein the second cleavable linker includes an enzyme-cleavable linker.
56. The method of paragraph 55, wherein the second cleavable linker includes a single stranded or double stranded nucleic acid.
57. The method of paragraph 56, wherein the single stranded or double stranded nucleic acid includes a recognition sequence for one or more restriction enzymes.
58. The method of any one of paragraphs 1-57, wherein the target protein-binding moiety is associated with the first oligonucleotide via a first cleavable linker and the one or more functional groups is associated with the first probe via a second cleavable linker, and optionally wherein the first and second cleavable linkers are cleaved by different mechanisms.
59. The method of any one of paragraphs 1-58, wherein the method further includes, prior to modifying a 3′ and/or 5′ end of the plurality of nucleic acids in step (b), fragmenting the plurality of nucleic acids in the biological sample.
60. The method of any one of paragraphs 1-59, wherein the one or more functional group(s) is associated with the target protein-binding moiety.
61. The method of any one of paragraphs 1-60, wherein the one or more functional group(s) is associated with the first oligonucleotide.
62. The method of paragraph 61, wherein the one or more functional group(s) is associated with 5′ end of the first oligonucleotide.
63. The method of paragraph 61 or 62, wherein the one or more functional group(s) is associated with 3′ end of the first oligonucleotide.
64. The method of any one of paragraphs 1-63, wherein the first oligonucleotide further includes a second oligonucleotide hybridized thereto, and wherein the one or more functional group(s) is associated with the second oligonucleotide.
65. The method of paragraph 64, wherein the one or more functional group(s) is associated with a 5′ end of the second oligonucleotide.
66. The method of paragraph 64 or 65, wherein the one or more functional group(s) is associated with a 3′ end of the second oligonucleotide.
67. The method of any one of paragraphs 1-66, wherein the one or more functional group(s) include one species of functional group.
68. The method of any one of paragraphs 1-66, wherein the one or more functional group(s) includes more than one species of functional group.
69. The method of any one of paragraphs 1-68, wherein one species of the one or more functional group(s) includes acrydite.
70. The method of any one of paragraphs 1-69, wherein step (h) includes determining
   (hi) the spatial barcode sequence or a complement thereof; and
   (hii) all or a portion of the second hybridized probe sequence or the complement thereof; and
   (hiii) the sequence of the target protein identification barcode.
71. The method of paragraph 70, wherein step (i) includes using the determined sequences of (hi), (hii) and (hiii).
72. The method of any one of paragraphs 1-71, further including, after step (a), substantially removing unbound first probe from the biological sample.
73. The method of any one of paragraphs 1-72, wherein the region complementary to the target nucleic acid is complementary to a poly(A) sequence, or a complement thereof.
74. The method of paragraph 73, wherein the region of the second probe complementary to the target nucleic acid is complementary to a coding region of an mRNA, or a complement thereof.
75. The method of any one of paragraphs 1-74, wherein the target protein includes one or more proteins selected from the group including Argonaute-1 (AGO-1), Argonaute-2 (AGO-2), Argonaute-3 (AGO-3), Argonaute-4 (AGO-4), P-element-induced wimpy testis (PIWI), Dicer, glycine/tryptophan (GW) repeats-containing 182 protein, heat shock protein 70 (Hsp70), and heat shock protein 90 (Hsp90).
76. The method of any one of paragraphs 1-75, wherein the target protein includes a protein that binds directly to and/or interacts directly with one or more protein selected from the group including Argonaute-1 (AGO-1), Argonaute-2 (AGO-2), Argonaute-3 (AGO-3), Argonaute-4 (AGO-4), P-element-induced wimpy testis (PIWI), Dicer, glycine/tryptophan (GW) repeats-containing 182 protein, heat shock protein 70 (Hsp70), and heat shock protein 90 (Hsp90).
77. The method of any one of paragraphs 1-75, wherein the target protein includes one or more proteins selected from the group including Hu-antigen R (HuR), heterogeneous nuclear ribonucleoprotein family (hnRNP), the arginine/serine-rich splicing factor protein family (SRSF), and RNA-binding motif (RBM) proteins.
78. The method of any one of paragraphs 1-75, wherein the target protein includes a protein that binds directly to and/or interacts directly with one or more protein selected from the group including Hu-antigen R (HuR), heterogeneous nuclear ribonucleoprotein family (hnRNP), the arginine/serine-rich splicing factor protein family (SRSF), and RNA-binding motif (RBM) proteins.
79. The method of any one of paragraphs 1-75, wherein the target protein includes the expression product of a gene selected from the group including TERT, ZC3H12C, ZNF106, TLR3, PIWIL2, APC, EXO1, RNASE4, SRSF12, RBM11, LRRFIP1, OASL, SMAD4, DKC1, ANG, SRSF5, KHDRBS2, ARHGEF28, SIDT2, RBPMS, INTS10, SECISBP2L, FTO, EIFIB, RNASE1, DICER1, EZH2, LARS2, ZCCHC2, ZFP36, EXOSC7, NOVA1, MEX3A, HINT3, RBMS3, RNASE6, PATL2, BYSL, ESRP2, CNOT8, RPL22L1, POLR2H, RNASE3, ELAVL2, MRPS36, PPARGC1B, ADAD2, CPEB4, PTBP1, CTIF, HBS1L, PNRC2, MRPL12, TDRD9, DYNC1H1, ADARB2, PDCD4, TEP1, DDX24, GFM1, TARBP1, ELAC1, RPL22, PTBP2, TARS, RBM7, BRIX1, ENOX1, SRRM4, CWF19L2, ZFP36L1, CLK4, DUSIL, HABP4, METTL1 and XPO4.
80. The method of any one of paragraphs 1-75, wherein the target protein includes a protein that binds directly to and/or interacts directly with the expression product of a gene selected from the group including TERT, ZC3H12C, ZNF106, TLR3, PIWIL2, APC, EXO1, RNASE4, SRSF12, RBM11, LRRFIP1, OASL, SMAD4, DKC1, ANG, SRSF5, KHDRBS2, ARHGEF28, SIDT2, RBPMS, INTS10, SECISBP2L, FTO, EIF1B, RNASE1, DICER1, EZH2, LARS2, ZCCHC2, ZFP36, EXOSC7, NOVA1, MEX3A, HINT3, RBMS3, RNASE6, PATL2, BYSL, ESRP2, CNOT8, RPL22L1, POLR2H, RNASE3, ELAVL2, MRPS36, PPARGC1B, ADAD2, CPEB4, PTBP1, CTIF, HBSIL, PNRC2, MRPL12, TDRD9, DYNC1H1, ADARB2, PDCD4, TEP1, DDX24, GFM1, TARBP1, ELAC1, RPL22, PTBP2, TARS, RBM7, BRIX1, ENOX1, SRRM4, CWF19L2, ZFP36L1, CLK4, DUSIL, HABP4, METTL1 and XPO4.
81. The method of any one of paragraphs 1-75, wherein the target protein includes the expression product of a gene selected from the group including RBM3, RBM4, RBM5, RBM6, RBM7, RBM8A, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM33, RBM34, RBM38, RBM39, RBM41, RBM42, RBM43, RBM44, RBM45, RBM46, RBM47, RBM48, RBM4B, RBMS1, RBMS2, RBMS3, RBMX, RBMX2, RBMXL1, RBMXL2, RBMXL3, RBMY1A1, RBMY1B, RBMY1C, RBMY1D, RBMY1E, RBMY1F, and RBMY1J.
82. The method of any one of paragraphs 1-75, wherein the target protein includes a protein that binds directly to and/or interacts directly with the expression product of a gene selected from the group including RBM3, RBM4, RBM5, RBM6, RBM7, RBM8A, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM33, RBM34, RBM38, RBM39, RBM41, RBM42, RBM43, RBM44, RBM45, RBM46, RBM47, RBM48, RBM4B, RBMS1, RBMS2, RBMS3, RBMX, RBMX2, RBMXL1, RBMXL2, RBMXL3, RBMY1A1, RBMY1B, RBMY1C, RBMY1D, RBMY1E, RBMY1F, and RBMY1J. 83. The method of any one of paragraphs 1-75, wherein the target protein includes a transcription factor or an enhancer binding protein.
84. The method of any one of paragraphs 1-75, wherein the target protein-binding moiety includes a plurality of target protein-binding moieties specific for a plurality of target proteins.
85. The method of any one of paragraphs 1-84 wherein the first probe further includes one or more functional domains.
86. The method of any one of paragraphs 1-85, wherein a second probe of the plurality of second probes further includes one or more functional domains.
87. The method of any one of paragraphs 26-32, wherein the first splint oligonucleotide further includes one or more functional domains.
88. The method of any one of paragraphs 1-87, wherein the capture probe further includes one or more functional domains.
89. The method of any one of paragraphs 85-88, wherein the one or more functional domains includes a Unique Molecular Identified (UMI), or a primer binding site, or a label, or dye.
90. The method of any one of paragraphs 1-89, wherein the region of the second probe complementary to one or more nucleic acids includes between about 4 and about 70 contiguous nucleotides, inclusive, optionally between about 8 and about 50 contiguous nucleotides, inclusive.
91. The method of any one of paragraphs 1-90, wherein the second probe hybridizes to the target nucleic acid in a region beginning 0-200 nucleotides, or any subrange thereof, relative to the end of a nucleic acid binding domain of the target protein.
92. The method of any one of the paragraphs 1-91, further including extending the hybridized second probe using the target nucleic acid as a template.
93. The method of any one of paragraphs 1-92, wherein the capture probe is included in an array including a plurality of capture probes.
94. The method of any one of paragraphs 1-93, wherein the capture probe and/or the combined probe include one or more binding sites for sequencing primers.
95. The method of any one of paragraphs 1-94, wherein the capture probe is associated with a substrate via a third linker.
96. The method of any one of paragraphs 1-95, wherein the capture probe is associated with a gel or a bead.
97. The method of paragraph 96, wherein the capture probe is associated with the gel or the bead via a third cleavable linker.
98. The method of paragraph 95 or 97, wherein the third cleavable linker is a photocleavable linker, UV-cleavable linker, or an enzyme-cleavable linker.
99. The method of any one of paragraphs 28 or 37 wherein the ligating step includes enzymatic ligation or chemical ligation.
100. The method of paragraph 99, wherein the ligating step includes T4 ligase-mediated ligation.
101. The method of any one of paragraphs 1-100, further including, prior to step (f) releasing the target nucleic acid from the combined capture probe.
102. The method of paragraph 101, wherein the releasing includes enzymic digestion of the target nucleic acid.
103. The method of any one of paragraphs 1-102, further including, prior to step (g) or (h), separating the combined probe from the target protein-binding moiety of the first probe.
104. The method of paragraph 103, wherein the separating includes cleavage of a first cleavable linker.
105. The method of any one of paragraphs 1-104, further including, prior to step (g) or (h), releasing the combined probe from the gel.
106. The method of paragraph 105, wherein the releasing includes cleavage of a second cleavable linker.
107. The method of any one of paragraphs 1-106, further including extending the combined probe using the capture probe as a template to generate an extended combined probe.
108. The method of any one of paragraphs 1-107, further including extending the capture probe using the combined probe as a template to generate an extended capture probe.
109. The method of paragraph 107 or 108, wherein the determining in step (h) includes amplifying all or part of the extended combined probe, or all or part of the extended capture probe, thereby generating an amplified product.
110. The method of paragraph 109, wherein the amplified product includes all or part of the sequence of the combined probe or a complement thereof, and the sequence of the spatial barcode, or a complement thereof.
111. The method of paragraph 109 or 110, wherein the determining in step (h) includes sequencing the amplified product.
112. The method of any one of paragraphs 1-111, wherein the target protein-binding moiety includes one or more functional domains.
113. The method of paragraph 112, wherein the functional domain includes a Unique Molecular Identified (UMI), or a primer site, or a label, or dye.
114. A releasable nucleic acid probe, including
   (i) a target protein-binding moiety and an associated first oligonucleotide including a non-embeddable second-probe docking sequence and, optionally, a target protein identification barcode, and
   (ii) one or more functional group(s) for gel embedding associated with the target protein-binding moiety or the end of the first oligonucleotide proximal thereto.
115. The nucleic acid probe of paragraph 114, wherein the target protein-binding moiety is associated with the first oligonucleotide via a first cleavable linker.
116. The nucleic acid probe of paragraph 115, wherein the first cleavable linker includes a photocleavable linker, UV-cleavable linker, or an enzyme-cleavable linker.
117. The nucleic acid probe of paragraph 115 or 116, wherein the first cleavable linker is an enzyme-cleavable linker.
118. The nucleic acid probe of any one of paragraphs 115-117, wherein the first cleavable linker includes single stranded or double stranded nucleic acid.
119. The nucleic acid probe of paragraph 118, wherein the single stranded or double stranded nucleic acid includes a recognition and/or cut sequence for one or more restriction enzyme.
120. A method of determining a target protein interaction with a target nucleic acid including:
   (a) contacting a biological sample including a plurality of nucleic acids with a plurality of first probes, wherein a first probe of the plurality of first probes includes
      (i) a target protein-binding moiety and an associated first oligonucleotide including a second-probe docking sequence and, optionally, a target protein identification barcode, and
      (ii) one or more functional group(s),
under conditions suitable for the target protein-binding moiety to bind to a target protein;
   (b) modifying a 3′ and/or 5′ end of the plurality of nucleic acids in the biological sample with one or more functional group(s);
   (c) embedding the biological sample in a gel, including forming an interaction between the one or more functional group(s) and the gel, wherein the target nucleic acid is included in the plurality of nucleic acids;
   (d) hybridizing a plurality of second probes to the plurality of nucleic acids to form a plurality of hybridized second probes,
   wherein a hybridized second probe of the plurality of hybridized second probes includes
      (i) a first-probe docking sequence; and
      (ii) a region complementary to one or more nucleic acids of the plurality of nucleic acids;
   (e) combining the first probe and the hybridized second probe to form a combined probe including a capture domain binding sequence,
   (f) hybridizing the capture domain binding sequence of the combined probe to the capture domain of a capture probe including
      (i) a spatial barcode and (ii) a capture domain; and
   (g) determining
      (gi) the spatial barcode sequence or a complement thereof;
      (gii) all or a portion of the hybridized second probe sequence or a complement thereof; and/or
      (giii) the sequence of the target protein identification barcode.
121. The method of any one of paragraphs 1 to 113, or 120, wherein the biological sample includes a tissue sample or tissue section.
122. The method of paragraph 121, wherein the tissue sample or tissue section includes a Formalin-Fixed Paraffin-Embedded (FFPE) tissue sample or tissue section.
123. The method of paragraph 122, wherein the tissue sample or tissue section includes a frozen and/or lyophilized tissue sample or tissue section.
124. The method of any one of paragraphs 121-123, wherein the method further includes, prior to step (a) one or more steps of de-crosslinking the tissue sample.
125. The method of paragraph 124, wherein the method further includes, prior to step (a), deparaffinizing the tissue sample.
126. The method of paragraph 125 wherein the deparaffinizing includes contacting the tissue sample with a solvent.
127. The method of any one of paragraphs 1-113, or 120-126, further including one or more steps of staining and/or labelling the biological sample.
128. The method of paragraph 127, wherein the one or more steps of staining and/or labelling the tissue sample is prior to step (a).
129. The method of paragraph 128, wherein the one or more steps of staining and/or labelling the tissue sample is during or after step (a), or during or after step (b) or during step (c).
130. The method of any one of paragraphs 127-129, wherein the staining the tissue sample includes hematoxylin and/or eosin (H and E) staining.
131. The method of any one of paragraphs 127-130, further including imaging the stained and/or labelled tissue sample, optionally further including de-staining the tissue sample.
132. A kit including
(a) a plurality of first probes, wherein a first probe of the plurality of first probes includes
   (i) a target protein-binding moiety and an associated first oligonucleotide including a non-embeddable second-probe docking sequence and, optionally, a target protein identification barcode, and
   (ii) one or more functional group(s) for gel embedding; and
(b) instructions for performing the method of any one of paragraphs 1-131.
133. The kit of paragraph 132, wherein a first probe of the plurality of first probes includes the releasable probe of any one of paragraphs 115-119.
134. The kit of paragraph 132 or 133, further including one or more of
(a) a plurality of second probes specific for a target nucleic acid, wherein a first probe of the plurality of first probes includes;
   (i) a first-probe docking sequence; and
   (ii) a region complementary to one or more ribonucleic acids of the plurality of nucleic acids;
(b) a solvent suitable to solubilize unbound first probes and/or unbound second probes;
(c) one or more Polymerase enzymes;
(d) one or more nuclease enzymes.
135. The kit of any one of paragraph 132 to 134, further including one or more first splint oligonucleotides,
wherein a splint oligonucleotide includes
   (i) a sequence complementary to a docking sequence of a first-probe; and
   (ii) a sequence complementary to a docking sequence of a second-probe.
136. The kit of any one of paragraphs 132-135, further including a plurality of capture probes, wherein a capture probe of the plurality of capture probes includes:
   (i) a spatial barcode; and
   (ii) a capture domain.
137. The method of any one of paragraphs 1-113 and 120-131, wherein embedding the biological sample in a gel further includes forming an interaction between the one or more functional group(s) in the plurality of nucleic acids and the gel.
138. The method of paragraph 137, wherein the target nucleic acid is RNA optionally mRNA, and
wherein the one or more functional group(s) includes a 5′-phosphate group or a 5′-phosphate group modified with a leaving group.
139. The method of paragraph 138, further including contacting the biological sample with an attachment agent, wherein the attachment agent includes
   (i) at least one reactive moiety capable of reacting with at least one 5′-phosphate group of the RNA or 5′-phosphate group of the RNA modified with a leaving group, and
   (ii) at least one attachment moiety capable of attaching covalently or noncovalently to a matrix-forming agent;
   forming a covalent bond between the reactive moiety of the attachment agent and the RNA;
contacting the biological sample with a matrix forming agent; and
forming a three-dimensional polymerized matrix from the matrix-forming agent, thereby embedding the biological sample and immobilizing the RNA in the three-dimensional polymerized matrix.
140. The method of paragraph 138 or 139, further including reacting at least one RNA in the biological sample with a polynucleotide kinase to provide the RNA including a 5′-phosphate group, and
optionally modifying the 5′-phosphate group with a leaving group to provide the RNA including a 5′-phosphate group modified with a leaving group,
optionally wherein the polynucleotide kinase is a T4 Polynucleotide Kinase (T4 PNK) or a T7 Polynucleotide Kinase (T7 PNK).
141. The method of paragraph 139 or 140, wherein the attachment agent is a compound of Formula (I)

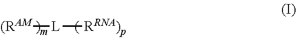

or a salt thereof,
wherein each R^{RNA} is, independently, a reactive moiety capable of reacting with at least one 5′-phosphate group of the RNA or 5′-phosphate group of the RNA modified with a leaving group;
wherein each RAM is independently an attachment moiety capable of attaching covalently or noncovalently to a matrix-forming agent;
wherein L is a bond or a linker moiety;
wherein m is an integer from 1 to 4, inclusive; and
wherein p is an integer from 1 to 4, inclusive.
142. The method of paragraph 141, wherein at least one reactive moiety of the attachment agent includes or is a nucleic acid oligonucleotide including from 2 to 20 nucleotide residues, inclusive,
optionally wherein the nucleic acid oligonucleotide is a DNA oligonucleotide.
143. The method of paragraph 142, wherein the method includes forming a covalent bond between a 3′-OH of the DNA oligonucleotide and the 5′-phosphate group of the RNA under catalysis of a ligase,
optionally wherein the ligase is T4 RNA Ligase 1.
144. The method of any one of paragraphs 139-142, wherein the method includes forming a covalent bond between the reactive moiety of the attachment agent and the 5′-phosphate group of the RNA without catalysis of an enzyme.
145. The method of any one of paragraphs 139-141, wherein the reactive moiety includes or is a nucleophilic group,
optionally wherein the reactive moiety includes or is —OH, or —SH, or —NH2.
146. The method of any one of paragraphs 139-145, wherein the attachment agent includes at least one attachment moiety capable of attaching covalently to a matrix-forming agent,
optionally wherein the at least one attachment moiety is or includes an alkenyl, alkynyl, allyl or vinyl moiety, ally ester moiety, an acrylamide moiety, an amide moiety, an alcohol moiety, an azide moiety, a polyol moiety, a furan moiety, a maleimide moiety, a norbornene moiety, a thiol moiety, a sulfide moiety, a phenol moiety, a urethane moiety, a cyano moiety, an amino moiety, an isocyanate moiety, an isothiocyanate moiety, an ether moiety, a dextran moiety, or an alginate moiety.
147. The method of any one of paragraphs 139-146, wherein at least one attachment moiety is or includes a click functional group,
optionally wherein at least one attachment moiety is or includes an azide moiety.
148. The method of paragraph 147, wherein the target nucleic acid is RNA, optionally mRNA, and
wherein the RNA is fragmented and includes a 2′,3′-vicinal diol.
149. The method of paragraph 148, further including contacting the biological sample with a formylation reagent,
wherein the formylation reagent converts the 2′,3′-vicinal diol moiety into 2′3′-dialdehyde moiety, thereby forming the one or more functional group(s).
150. The method of paragraph 149, further including
(i) contacting the biological sample with an attachment agent including at least one aldehyde-reactive group capable of reacting with at least one aldehyde of the 2′,3′-dialdehyde moiety of the fragmented ribonucleic acid to form a covalent bond and at least one attachment moiety capable of reacting with a matrix-forming agent to form a covalent bond;
(ii) contacting the biological sample with a matrix-forming agent; and
(iii) forming a three-dimensional polymerized matrix from the matrix-forming agent, thereby embedding the biological sample in the three-dimensional polymerized matrix and anchoring the fragmented RNA to the three-dimensional polymerized matrix.
151. The method of any one of paragraphs 148-150 further including contacting the biological sample including the fragmented RNA with a 3′ phosphatase to provide the fragmented RNA including a 2′,3′-vicinal diol.
152. The method of paragraph 151, wherein the fragmented RNA has a 2′,3′cyclo-phosphate fragmentation at 3′-terminal end, or a 2′ hydroxyl and a 3′ phosphate fragmentation at 3′-terminal end, and
wherein 3′ phosphatase catalyzes the formation of the 2′,3′-vicinal diol, optionally wherein 3′ phosphatase is T4 polynucleotide kinase.
153. The method of any one of paragraphs 150-152, wherein the attachment agent is N-(2-aminoethyl) methacrylamide, 2-aminoethyl methacrylate, or 2-aminoethyl (E)-but-2-enoate.
154. The method of any one of paragraphs 150-153, further including contacting the biological sample with a reducing agent, optionally wherein the reducing agent is sodium borohydride.
155. The method of any one of paragraphs 141-154, wherein the matrix-forming agent is selected from acrylamide, bisacrylamide, cellulose, alginate, polyamide, agarose, dextran, or polyethylene glycol, or a combination thereof.
156. The method of any one of paragraphs 141-155, wherein the three-dimensional polymerized matrix is formed by subjecting the matrix-forming agent to polymerization.
157. The method of paragraph 156, wherein the polymerization is initiated by adding a polymerization-inducing catalyst, or exposing UV light or functional cross-linkers to the biological sample.

The entire contents of all the references (including literature references, issued patents, published patent applications, and co pending patent applications) cited throughout this application are hereby expressly incorporated by reference.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific forms of the invention described herein. Such equivalents are intended to be encompassed by the following claims.

We claim:

1. A method of determining a location and/or abundance of an interaction between a target protein and a target nucleic acid in a biological sample, the method comprising:
(a) contacting a biological sample comprising a plurality of nucleic acids with a plurality of first probes, wherein a first probe of the plurality of first probes comprises
(i) a target protein-binding moiety and a first oligonucleotide comprising a second-probe docking sequence and, optionally, a target protein identification barcode, and
(ii) one or more functional group(s),
under conditions suitable for the target protein-binding moiety to bind to a target protein;
(b) modifying a 3′ and/or 5′ end of the plurality of nucleic acids in the biological sample with one or more functional group(s),
wherein the target nucleic acid is comprised in the plurality of nucleic acids;
(c) embedding the biological sample in a gel, comprising forming an interaction between the one or more functional group(s) and the gel;
(d) hybridizing a plurality of second probes to the plurality of nucleic acids to form a plurality of hybridized second probes,
wherein a hybridized second probe of the plurality of hybridized second probes comprises
(i) a first-probe docking sequence;
(ii) a region complementary to the target nucleic acid; and
(iii) a capture domain binding sequence;
(e) combining the first probe and the hybridized second probe to form a combined probe comprising the capture domain binding sequence,
(f) releasing the combined probe;
(g) hybridizing the capture domain binding sequence of the combined probe to a capture domain of a capture probe,
wherein the capture probe comprises
(i) a spatial barcode, and (ii) the capture domain; and
(h) determining
(hi) the spatial barcode sequence or a complement thereof;
(hii) all or a portion of the hybridized second probe sequence or a complement thereof; and/or
(hiii) the sequence of the target protein identification barcode; and
(i) using the determined sequences of (hi), (hii) and optionally (hiii) to identify the location and/or abundance of the interaction between the target protein and target nucleic acid in the biological sample.

2. The method of claim 1, wherein the target nucleic acid is DNA or RNA,
optionally wherein the RNA is selected from the group consisting of small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (incRNA), and transfer RNA (tRNA).

3. The method of claim 1, further comprising one or more steps of fragmenting the plurality of nucleic acids,
optionally wherein the plurality of nucleic acids is fragmented prior to step (a), or wherein the fragmenting occurs after or during any one of steps (a), (b), (c), (d) or (e).

4. The method of claim 3, wherein the fragmenting comprises contacting the biological sample with one or more nuclease enzymes.

5. The method of claim 1, further comprising permeabilizing the biological sample,
optionally wherein the permeabilizing comprises contacting the biological sample with a permeabilization reagent selected from the group consisting of an endopeptidase, a protease, sodium dodecyl sulfate (SDS), polyethylene glycol tert-octylphenyl ether, polysorbate 80, polysorbate 20, N-lauroylsarcosine sodium salt solution, and saponin.

6. The method of claim 1, wherein step (f) and/or step (g) further comprises degrading or otherwise removing the target nucleic acid hybridized to the combined probe and/or releasing the combined probe from the functional group(s),
optionally wherein the degrading or otherwise removing comprises contacting the biological sample with a nuclease.

7. The method of claim 6, wherein the target nucleic acid is RNA and wherein the nuclease is an RNase H.

8. The method of claim 1,
wherein the plurality of second probes comprises a library of sequences complementary to all or part of each nucleic acid of the plurality of nucleic acids.

9. The method of claim 1, wherein the first-probe docking sequence is substantially complementary to the second-probe docking sequence, and
wherein combining the first probe and the hybridized second probe comprises hybridizing the first-probe docking sequence with the second-probe docking sequence.

10. The method of claim 1, wherein combining the first probe and the hybridized second probe to form a combined probe comprising a capture domain binding sequence in step (e) comprises ligating the first probe and second hybridized probe together using a first splint oligonucleotide;
wherein the first splint oligonucleotide comprises
(i) a sequence substantially complementary to the first-probe docking sequence; and
(ii) a sequence substantially complementary to the second-probe docking sequence.

11. The method of claim 10, wherein the sizes and/or sequences of the first-probe docking sequence, second-probe docking sequence, and/or the first splint oligonucleotide are configured so that combining the first probe and hybridized second probe in step (e) preferably occurs when the target protein is bound directly to the target nucleic acid.

12. The method of claim 1, wherein the capture probe is comprised in a spatial array comprising a plurality of capture probes, optionally wherein the array comprises a plurality of features comprising the plurality of capture probes.

13. The method of claim 1, wherein the hybridized second probe comprises a first and a second RTL oligonucleotide,
wherein each of the first and second RTL oligonucleotides comprises a sequence complementary to the target nucleic acid, and
wherein hybridizing a plurality of second probes to the plurality of nucleic acids in step (d) comprises
(i) hybridizing the first RTL probe with the target nucleic acid;
(ii) hybridizing a second RTL probe with the target nucleic acid; and
(iii) ligating the first and second RTL probes together to form the hybridized second probe.

14. The method of claim 1, wherein the biological sample is a tissue section, optionally a fresh frozen tissue section or a fixed tissue section.

15. The method of claim 1, wherein, wherein the target protein-binding moiety comprises a nucleic acid,
optionally wherein the nucleic acid comprises an aptamer,
wherein the nucleic acid comprises the target protein identification barcode.

16. The method of claim 14, wherein the target protein-binding moiety comprises a polypeptide or protein,
optionally wherein the target protein binding moiety comprises an immunoglobulin, or antigen-binding fragment thereof.

17. The method of claim 1, wherein embedding the biological sample in a gel further comprises forming an interaction between the one or more functional group(s) in the plurality of nucleic acids and the gel,
wherein the target nucleic acid is RNA, optionally mRNA, and
wherein the one or more functional group(s) comprises a 5′-phosphate group, a 5′-phosphate group modified with a leaving group, acrydite, or a 3′ diol.

18. The method of claim 17, further comprising contacting the biological sample with an attachment agent, wherein the attachment agent comprises
(i) at least one reactive moiety capable of reacting with at least one 5′-phosphate group of the RNA or 5′-phosphate group of the RNA modified with a leaving group, and
(ii) at least one attachment moiety capable of attaching covalently or noncovalently to a matrix-forming agent;
forming a covalent bond between the reactive moiety of the attachment agent and the RNA;
contacting the biological sample with the matrix-forming agent; and
forming a three-dimensional polymerized matrix from the matrix-forming agent, thereby embedding the biological sample and immobilizing the RNA in the three-dimensional polymerized matrix.

19. The method of claim 1, further comprising extending the capture probe using the combined probe as a template, thereby generating an extended capture probe; and/or extending the combined probe using the capture probe as a template.

20. The method of claim 19, wherein the determining in step (h) comprises sequencing the extended capture probe, or a complement thereof.
